(19) United States
(12) Patent Application Publication
(10) Pub. No.: US 2025/0025473 A1
(43) Pub. Date: Jan. 23, 2025

(54) CELL IMPERMEABLE KINASE INHIBITORS TO TREAT SARS-COV-2 AND/OR HSV VIRAL INFECTIONS

(71) Applicant: Albert Einstein College of Medicine, Bronx, NY (US)

(72) Inventors: Betsy C. Herold, Bronx, NY (US); Steven C. Almo, Bronx, NY (US)

(21) Appl. No.: 18/707,839

(22) PCT Filed: Nov. 11, 2022
(86) PCT No.: PCT/US2022/049665

§ 371 Date: May 6, 2024

Related U.S. Application Data
(60) Provisional application No. 63/407,943, filed on Sep. 19, 2022.
(60) Provisional application No. 63/279,035, filed on Nov. 12, 2021.

(51) Int. Cl. A61K 31/553; A61K 45/06; A61P 31/14; A61P 31/22

(52) U.S. Cl. CPC A61K 31/553; A61K 45/06; A61P 31/14; A61P 31/22

(57) ABSTRACT

Methods of preventing or treating a coronavirus infection or a herpes simplex virus infection in a subject or preventing or treating a disease caused by a coronavirus infection or a herpes simplex virus infection in a subject are disclosed. The methods comprise administering to the subject an effective amount of a cell-impermeable inhibitor comprising a cell-impermeable kinase inhibitor, a cell-impermeable Akt inhibitor, an anti-Akt antibody, a cell-impermeable 3-phosphoinositide-dependent protein kinase-1 (PDPK1) inhibitor, an anti-PDPK1 antibody, or a combination thereof.

### CROSS-REFERENCE TO RELATED APPLICATION

This application claims the benefit of U.S. Provisional Application No. 63/407,943 filed Sep. 19, 2022 and U.S. Provisional Application No. 63/279,035 filed on Nov. 12, 2021, which are incorporated herein in their entirety.

### FEDERAL RESEARCH STATEMENT

This invention was made with government support under grant numbers IS100D023591-01, R01AI134367, P30 AI124414, awarded by the National Institutes of Health. The government has certain rights in the invention.

### BACKGROUND

Severe acute respiratory syndrome coronavirus-2, or SARS-CoV-2, is the name given to the coronavirus that causes the respiratory disease called coronavirus disease 19 (COVID-19). SARS-CoV-2 was first known to infect humans in 2019. Despite efforts to contain the disease in China, the virus has spread around the world, and COVID-19 was declared to be a pandemic by the World Health Organization (WHO) in March 2020. COVID-19 primarily spreads through the respiratory tract by droplets, respiratory secretions, and direct contact. SARS-CoV-2 is highly transmissible in humans and can cause severe disease, particularly in the elderly and people with underlying chronic diseases.

Currently, there are limited antiviral treatments or therapies for COVID-19. In October 2020, Veklury (remdesivir) was approved for use in adult and pediatric patients 12 years of age and older weighing at least 40 kg for the treatment of COVID-19 requiring hospitalization. The use of remdesivir is only authorized in a hospital or healthcare setting capable of providing acute care comparable to inpatient hospital care. Monoclonal antibodies have also received emergency use authorization to treat patients with COVID-19. Merck has evaluated molnupiravir, a prodrug of the nucleoside derivative that resembles cytidine and introduces copying errors during RNA viral replication, as a candidate oral antiviral agent. The protease inhibitor paxlovid, has also been evaluated by Pfizer for treatment of non-hospitalized patients with SARS-CoV-2, which in an interim analysis reduced the risk of hospitalizations and deaths. Notably neither prevents viral entry.

Several vaccines against SARS-CoV-2 have been developed and approved, however, their long-term efficacy is unknown, particularly in the face of emerging variants of the virus.

Herpes simplex virus serotypes 1 and 2 (HSV-1 and HSV-2) are major global health problems, representing leading causes of infectious corneal blindness, sporadic fatal encephalitis and significant perinatal disease. Acyclovir and its related prodrugs are converted intracellularly to acyclovir triphosphate, which inhibits HSV viral DNA synthesis, but resistance and toxicities underscore the need to identify new targets and new classes of antiviral drugs (S. S. Wilson, et al, Expert Rev Anti Infect Ther 7, 559-568 (2009)).

SARS-CoV-2 as well as HSV-1 and HSV-2 infections remain a threat to public health. There thus remains a need for antiviral treatments which can effectively treat and/or prevent SARS-CoV-2 and the disease COVID-19 caused by the SARS-CoV-2 virus. There also remains a need for antiviral treatments which can effectively treat and/or prevent HSV infections and the diseases caused these viruses.

### SUMMARY

The present disclosure provides a method of preventing or treating a coronavirus infection in a subject or preventing or treating a disease caused by a coronavirus infection in a subject, comprising administering to the subject an effective amount of a cell-impermeable inhibitor comprising a cell-impermeable kinase inhibitor, a cell-impermeable Akt inhibitor, a cell-impermeable 3-phosphoinositide-dependent protein kinase-1 inhibitor, and/or a combination thereof.

Also disclosed is a method of preventing or treating a condition, a disorder or a disease associated with a SARS-CoV-2 infection in a subject comprising administering to the subject an effective amount of a cell-impermeable inhibitor, comprising a cell-impermeable kinase inhibitor, a cell-impermeable Akt inhibitor, a cell-impermeable 3-phosphoinositide-dependent protein kinase-1 inhibitor, or a combination thereof.

Disclosed herein also is a method of disrupting or reducing infection of a cell by a coronavirus, comprising contacting the cell with an effective amount of a cell-impermeable inhibitor comprising a cell-impermeable kinase inhibitor, a cell-impermeable Akt inhibitor, a cell-impermeable 3-phosphoinositide-dependent protein kinase-1 inhibitor, or a combination thereof.

The present disclosure provides a pharmaceutical composition comprising a pharmaceutically acceptable carrier and an amount of a cell-impermeable inhibitor comprising a cell-impermeable kinase inhibitor, a cell-impermeable Akt inhibitor, a cell-impermeable 3-phosphoinositide-dependent protein kinase-1 inhibitor, or a combination thereof for inhibiting or treating a coronavirus infection in a subject or for preventing or treating a disease caused by a coronavirus infection in a subject.

Also disclosed is a method of preventing or treating a herpes simplex virus infection in a subject or preventing or treating a disease caused by a herpes simplex virus infection in a subject, comprising administering to the subject an effective amount of a cell-impermeable inhibitor comprising a cell-impermeable kinase inhibitor, a cell-impermeable Akt inhibitor, a cell-impermeable 3-phosphoinositide-dependent protein kinase-1 inhibitor, or a combination thereof.

The above described and other features are exemplified by the following figures and detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-D. Synthesis of cell impermeable staurosporine analog (CIMSS) and the effects of the parental drug and the analog on cell proliferation and viability. (1A): Scheme illustrating the synthetic route to the sodium salt of CIMSS. The secondary amine of staurosporine was derivatized via amidation of succinic anhydride and the resulting carboxylate (compound 5) was condensed with aminomethyl-triazol-propane-sulfonate (compound 4, which was obtained in four steps from NBoc-propargylamine and azidopropanol) to afford CIMSS-Na in modest yield as a white powder. (1B): HaCat cells (˜50% confluence) were cultured in media containing increasing concentrations of staurosporine, CIMSS, or the equivalent concentration of DMSO, and cell proliferation quantified after 24 and 72 h using the Cell Titer 96 Aqueous One Solution Cell Proliferation Assay. (1C): Confluent HaCat cells were cultured in media containing 10 μM CIMSS, 10 μM staurosporine or 0.1% DMSO (control) and viability assessed after 24, 72 and 120 h. Note that staurosporine was completely cytotoxic after 72 and 120 hours of exposure and thus no bar is visible. (1D): Primary vaginal epithelial cells (˜50% confluent) were cultured in media containing 0.5% DMSO, 10 or 50 μM CIMSS or 1 or 10 μM staurosporine for 120 h and cell proliferation and viability monitored. Results are presented as mean±SEM odu as a percentage of the DMSO control (n=2 independent experiments each conducted in duplicate for C and 1 experiment conducted in duplicate for D). Results in B-D were compared by ANOVA, *p<0.05, **p<0.01, ***p<0.001, ****p<0.0001).

FIGS. 2A-D. CIMSS does not induce apoptosis or block phosphorylation of Akt intracellularly in response to insulin. HaCat cells were exposed to 0.1% DMSO, 10 μM CIMSS or 10 μM staurosporine and after 6 or 24 hours of incubation, the cells were fixed and stained for activated caspases (red), integrity of plasma membrane with SYTOX Green, and nuclei (Hoechst stain, blue). (2A): Representative images are taken with ZeissLive/DuoScan (objective 100×1.4, bar=10 μm). (2B): the percentage of cells positive for activated caspase and SYTOX Green was quantified after counting ˜100 cells from 4 independent fields, n=3 experiments; asterisks indicate significance relative to DMSO (unpaired t-test, ***p<0.001; ****p<0.0001). (2C): HaCat cells were exposed to 0.1% DMSO, 0.1, 1, or 10 uM CIMSS or 0.01, 0.1, 1, or 10 μM staurosporine and after 8 hours of incubation, lysates were prepared and analyzed by Western blotting for cleaved PARP-1 or cleaved caspase 8 (p43/41). The intensity of cleaved PARP-1 or caspase 8 (relative to β-actin) is indicated below each lane. The immunoblot is representative of 2 independent experiments. (2D): HaCat cells were exposed to insulin (10 μM) in the absence or presence of 10 or 100 μM CIMSS or 0.1 μM staurosporine for 30 and 120 min, fixed with or without Triton-X, stained for nuclei (blue), pAkt^{T308} (red) or total Akt (green). Representative images from two independent experiments obtained with Leica SP8 microscope equipped with objective 63X1.4 are shown (bar=10 um).

FIGS. 3A-D. CIMSS inhibits HSV infection. (3A): HaCat (n=4) or primary vaginal cells (n=2) were treated with increasing doses of CIMSS (or DMSO control) and then infected with HSV-2(G). Plaques were counted by immunostaining after 48 h and are presented as the percent reduction in viral plaque numbers relative to the DMSO controls, which had ˜200 plaques per well (mean±SD) (***p<0.001, ***p<0.0001, ANOVA with multiple comparisons compared to DMSO control for HaCat cells). (3B): HaCat cells were synchronously infected with HSV-2 (G), treated with media containing 10 μM CIMSS or 0.1% DMSO at temperature shift, extracellular virus was inactivated with low pH citrate buffer and cells were overlaid with methylcellulose. Plaques were counted at 48 h (mean±SEM, n=2). Asterisks indicate significance at each time point by ANOVA (**** p<0.0001). (3C): HaCat or primary vaginal epithelial cells were mock or synchronously infected with HSV-2(G) (MOI=10 pfu/cell) and 0.1% DMSO, 10 μM CIMSS, 2 μg/ml rabbit anti-Akt or a control IgG were added at the time of temperature shift. Nuclear extracts were prepared after 1 h incubation at 37° C. and probed with antibodies for VP16, histone-1 (nuclear protein) or Golgin-1 (cytoplasmic protein). The blot is representative of results obtained in 2 independent experiments; intensity of VP16 band (relative to histone-1) is indicated below each lane. (3D): CIMSS and anti-Akt antibodies inhibit HSV capsid entry. HaCat cells were synchronously infected with HSV-1K26GFP, which expresses a green fluorescent protein fused to the capsid protein VP26 (MOI 10 PFU/cell), with the addition of 0.1% DMSO, 10 μM CIMSS, 2 μg/ml polyclonal anti-Akt antibody or 10 μg/ml of cycloheximide at the time of temperature shift. After 1 or 4 hours of incubation, the cells were fixed. Plasma membranes were stained using Image-IT™ LIVE Plasma Membrane kit and nuclei were stained with DAPI. The percentage of GFP+ cells was determined by counting ˜200 cells over 3-4 fields. Images were obtained with Leica SP8 microscope, objective 63×1, bar=18 μm; xz images were captured with optical slice of 0.6 μm, 25-30 slices per image (***p<0.001 and ****p<0.0001 compared to DMSO, ANOVA).

FIGS. 4A-E. CIMSS inhibits HSV entry downstream of viral binding, activation of phospholipid scramblase, and translocation of phosphatidylserines and Akt to the outer leaflet of the plasma membrane. (4A): HaCat cells were exposed to HSV-2(G) at the indicated multiplicities of infection (pfu/cell) (MOI) of HSV-2(G) in the absence or presence of 10 μM CIMSS for 4 hours at 4° C. The cells were then washed, lysed and Western blots of cell lysates prepared and probed with a mAb to gD as a marker of cell-bound virus and anti-3-actin as a loading control. The blot is representative of results obtained in 2 independent experiments; relative intensity of gD band is shown beneath each lane. (4B): HaCat cells were loaded with Fura-2 and then infected with purified HSV-2(G) (10 pfu/cell), or mock-infected in the presence of control buffer (DMSO, 0.1%) or CIMSS (10 μM) and the kinetics of calcium response monitored. Representative responses are shown for the first 30 minutes (left) and the mean calcium released over the first 3 minutes and then the extended response in the first hour following viral exposure were calculated from 4 wells in 3 independent experiments, each containing 5×104 cells (right). Asterisks indicate statistically significant differences by ANOVA (*, p<0.05, ** p<0.01, *** p<0.001) relative to control cells (mock-infected, DMSO). (4C): HaCaT cells were mock-infected or infected with HSV-2(G) in the presence of 0.1% DMSO, 10 μM CIMSS or 10 μM staurosporine for 30 minutes and then the cells were lysed and incubated with rabbit or goat anti-PLSCR antibody and immune complexes precipitated with protein A-agarose and analyzed by Western blotting with a mouse anti-phosphotyrosine (PY20) or mouse anti-PLSCR mAb. The blot is representative of results obtained in 3 independent experiments. (4D): HaCat cells were infected with HSV-2(G) (moi of 10 pfu/cell) in the presence of 10 μM CIMSS, 10 μM staurosporine or 0.1% DMSO, and prior to infection (time=0 minutes) or after 15, 30 or 120 minutes, the cells were fixed without permeabilization and stained with mAbs for phosphatidylserine (PtdS) (left panel) or Akt (right panel). Nuclei were stained blue with DAPI. Images are representative of 2 independent experiments. (4E): HaCat cells were infected with HSV-2(G) (moi 10 pfu/cell) in the presence of 0.1% DMSO or 10 μM CIMSS and at the indicated times, cells were fixed without (-Triton) or with (+Triton) permeabilization and stained with mAbs for phosphorylated Akt (pAkt^{S473}, red) and pAkt^{T308}, green). Nuclei are stained blue (DAPI). Images were obtained with ZeissLive/DuoScan (objective 100×1.4, bar=10 μm) are representative of 2 independent experiments; quantification is shown in FIG. 11.

FIGS. 5A-B. HSV triggers translocation of 3-phosphoinositide-dependent protein kinase 1 (PDPK1) and PLCγ1 to the outer leaflet and their subsequent phosphorylation is inhibited by CIMSS. (5A): HaCat cells were mock-infected or synchronously infected with HSV-2(G) in the absence or presence of 10 μM CIMSS. After 15 minutes incubation, cell surface proteins were biotinylated and precipitated with streptavidin magnetic beads and analyzed by immunoblotting with Abs to pPDPK1^{S241} and total PDPK1, pPLCγl^{Y783} and total PLCγl, pAkt^{T308} and total Akt, and FIC-1 (cytosolic protein); controls include whole cell lysates. Results are representative of 2 independent experiments. (5B): HaCat cells were mock-infected or infected with HSV-2(G) (10 pfu/cell) in the presence of 0.1% DMSO or 10IM CIMSS and at the indicated time post-infection cells were fixed with or without Triton-X permeabilization and stained for pPDPK1S241 (red, upper panel) or pPLCγl^{Y783} (green, lower panel). Representative images from 2-3 independent experiments are shown; bars represent 10 μm.

FIGS. 6A-D. PDPK1 is phosphorylated upstream and PLCγ downstream of Akt phosphorylation at the outer leaflet of the plasma membrane. (6A): HaCat cells were transfected with siControl or siAkt and silencing assessed by preparing Western blots after 72 h and probing for Akt and P-actin; blots are representative of 2 independent experiments and relative Akt expression after scanning images is shown. (6B): The siRNA-transfected cells were exposed to HSV-2(G) (10 pfu/cell) (72 h after transfection) and at the indicated times post-infection (0, 30 or 120 minutes), the cells were fixed and stained (with or without Triton X permeabilization) with antibodies to detect Akt (green), pPDPK1^{S241} (red) or pPLCγl^{Y783} (red); nuclei were stained blue with DAPI. Images are representative of 2 independent experiments (Leica SP8, objective 63×1.4, bar=10 um). (6C): The siRNA transfected cells were infected with HSV-2(G) (100-200 pfu/well) and plaques were counted at 48 h; results are presented for 2 independent experiments each performed in duplicate as percent of pfu/well detected in the siControl transfected cells. The asterisks indicate p<0.0001, unpaired t-test. (6D): HaCat cells were infected with HSV-2(G) (10 pfu/cell) in the presence of 10 U/ml of apyrase or control media and at the indicated times post-infection, fixed and stained (without permeabilization) with antibodies to detect phosphatidylserines (PtDS) and phospholipid scramblase (PLSCR1), or (FIG. 6B) pAkt^{T308} and pPDPK1^{S241}. Images (Leica SP8, objective 63×1.4, bar=10 um) are representative fields from 2 independent experiments.

FIGS. 7A-E. CIMSS inhibits VSV pseudotyped with SARS-CoV-2 spike protein and native SARS-CoV-2 infection. (7A). Vero cells were infected with VSV viruses expressing the enhanced green fluorescence protein and VSV glycoprotein G (VSV-G), SARS-CoV-2 spike protein (VSV-S) or Ebola virus glycoprotein (VSV-EBOV-GP) in the presence of 0.1% DMSO or 10 μM CIMSS. The percentage of GFP-positive cells were quantified 24 hours after infection by microscopy and results are shown as mean±SD from 2 independent experiments with ˜200-300 cells counted over 4 fields per experiment (n=4 for VSV-G), ***p<0.0001 comparing CIMSS versus DMSO for each virus, unpaired t-test. Representative images are shown on the right, images with VSV-G were obtained with ZeissLive/DuoScan (objective 100×1.4) and for VSV-S and VSV-EBOV-GP with Leica SP8 (objective 63×1.4) (bar=10 μm). (7B): Western blots of Vero or Huh7 cell lysates probed for TMPRSS2 and β-actin as loading control. The intensity of TMPRSS2 relative to β-actin is indicated below each lane; blots are representative of 2 independent experiments. Vero, Huh7 or Calu-3 cells were infected with the VSV-S in the presence of 0.5% DMSO or increasing concentrations of CIMSS (FIG. 7C) or camostat mesylate (FIG. 7D) and plaques counted after 48 h infection or, for Calu-3 cells, culture supernatants were harvested and infectious viral yields quantified by titering on Vero cells. Asterisks indicate significance relative to the DMSO control for each virus (ANOVA, *p<0.05, ** p<0.01, *** p<0.001 and ****p<0.0001). Results are mean±SD from 2-4 independent experiments conducted in duplicate. (7E): Huh-7.5 cells were treated with CIMSS 1 h prior to infection with SARS-CoV-2 (strain: WA1/2020). Infections were performed at MOIs of 0.25 PFU/cell for a 24 h timepoint and 0.01 PFU/cell for a 72 h timepoint. Cells were subsequently stained for the nucleocapsid protein; nuclei were stained using Hoechst 33342. The percentage of SARS-CoV-2 positive cells and nuclei (cell number) are presented relative to the DMSO control. Error bars represent SEM for n=3 replicates.

FIGS. 8A-C. VSV-S triggers activation of phospholipid scramblase (PLSCR) and the translocation of phosphatidylserines. (8A): Vero cells were mock-infected or infected with the indicated viruses for 30 minutes in the absence or presence of CIMSS or staurosporine (10 μM each) or murine anti-ACE2 (anti-ACE), anti-Spike (anti-S) or an isotype control IgG (10 pg/ml of each immunoglobulin). The cells were lysed and incubated with rabbit anti-PLSCR1 antibody and immune complexes precipitated with protein A-agarose and analyzed by Western blotting with a mouse anti-phosphotyrosine (PY20) or mouse anti-PLSCR mAb. The blot is representative of results obtained in 2 independent experiments. (8B): Vero cells were stained for plasma membranes (green) with wheat germ agglutinin conjugated with Alexa488 and then mock-infected or infected with the indicated viruses in the presence of 0.1% DMSO or 10 μM CIMSS and after 30 minutes, 1 hour or 4 hours, fixed and stained with an antibody to phosphatidylserines (PtdS) (red); nuclei were stained with DAPI. Images were obtained with Leica SP8 (objective 63×1.4, bar=10 um) (quantification, See FIG. 11; blue). (8C): Vero cells were mock-infected or infected with the indicated VSV pseudotyped viruses in the absence or presence of 10 μM CIMSS. After 15, 30 or 60 minutes, the cell surface proteins were biotinylated and precipitated with streptavidin magnetic beads and analyzed by immunoblotting with Abs to pAkt^{T308} and total extracellular Akt. Results are representative of 2 independent experiments.

FIGS. 9A-D. Silencing of PDPK1, PLCγ1 and Akt inhibits VSV-S infection. (9A): Vero cells were transfected with control siRNA or siRNA targeting Akt, PDPK1, PLCγ or FIC-1 and after 72 h, cell lysates were assayed by preparing Western blots and probing for respective protein or β-actin as a loading control. Blots were scanned and the percent reduction in protein expression of the silenced protein relative to siControl-transfected cells is indicated; blots are representative of two independent experiments. (9B): Silenced cells were infected in duplicate with VSV-G or VSV-S and plaques quantified after 48 h incubation. Results are presented as mean±SD and asterisks indicate significance relative to plaques formed on siControl wells (ANOVA, **** p<0.0001). (9C): Vero cells were transfected with siPDPK1 or control siRNA as in FIG. 10A and then infected with VSV-G or VSV-S. Following incubation for 30, 60 or 120 minutes, cells were fixed without or with Triton-X permeabilization and stained with antibodies to pAkt^{T308} (red) or PDPK1 (green); nuclei were stained blue with DAPI. Images are representative of results obtained in 2 independent experiments. (9D): Vero cells were infected with VSV-S or VSV-G in the presence of antibodies that target Akt (rabbit polyclonal), PDKP1 (rabbit polyclonal), the ACE2 receptor (murine monoclonal), or a murine monoclonal isotype control for 1 hour, washed, and infection monitored by counting plaques 24 h pi. Results are presented as mean±SD and asterisks indicate significance relative to plaques formed on isotype control antibody treated wells (**p<0.01, ***p<0.001, ****p<0001).

FIG. 10. VSV-Spike triggers translocation of Akt, PDPK1 to the outer leaflet and the phosphorylation of these kinases is inhibited by CIMSS. Vero cells were infected with VSV-S or native VSV in the presence of 0.1% DMSO or 10 mM CIMSS and at baseline (t=0 minutes) or 30 and 60 minutes following infection, the cells were fixed with or without Triton-X permeabilization and stained with conjugated antibodies to phosphorylated Akt, phosphorylated PDPK1, total Akt or total PDPK1; nuclei were stained blue with DAPI. Results are representative of 2 independent experiments and were obtained with Leica SP8 microscope (objective 63×1.4).

FIGS. 11A-C. Quantification of confocal images. Confocal images were scanned and the mean fluorescence intensity of phosphatidylserines and Akt from FIG. 4D (FIG. 11A), phosphorylated Akt (pAktS473 and pAktT308) from non-permeabilized and permeabilized cells from FIG. 4E (FIG. 11B), and phosphatidylserines from FIG. 8B (FIG. 11C) were quantified after counting 200-300 cells over 4 independent fields.

FIG. 12. CIMSSNa analogs may be prepared with a variety of linkers. For example, the amide coupling at the staurosporine can be replaced with an amine, the length of the carbon chain between the staurosporine and the linker amide can be varied, and the methyl triazole can be omitted.

FIG. 13A-B. Model of how cell-impermeable kinase inhibitors block viral entry. (13A) Binding of HSV to cellular receptors (1) triggers intracellular calcium transients (2), which activate phospholipid scramblase-1 (3) leading to the translocation of phosphatidylserines and cellular (PDPK1, Akt, PLCg and possibly others) to the outer leaflet of the plasma membrane (4) where, analogous to the canonical cytoplasmic signaling pathways, autophosphorylation of PDPK1 triggers phosphorylation of outer leaflet Akt, which, in turn, phosphorylates PLCg (5) This signaling pathway is required for HSV entry and is associated with subsequent restoration of phospholipid distribution (6). (13B) Cell-impermeable kinase inhibitor (e.g., CIMSS) or antibodies to Akt block the activation of this extracellular signaling pathway (5) and prevent HSV entry resulting in (6) persistence of extracellular PtdS, which may lead to apoptosis.

### DETAILED DESCRIPTION

Described herein are methods of inhibiting or treating a coronavirus infection in a subject or preventing or treating a disease caused by a coronavirus infection in a subject. The methods include administering to the subject an effective amount of a cell-impermeable inhibitor. The cell-impermeable inhibitor includes a cell-impermeable kinase inhibitor, a cell-impermeable Akt inhibitor, a cell-impermeable 3-phosphoinositide-dependent kinase-1 inhibitor, or a combination thereof.

Also disclosed are methods of inhibiting or treating a herpes simplex virus (HSV) infection in a subject or preventing or treating a disease caused by HSV infection in a subject. The methods include administering to the subject an effective amount of a cell-impermeable 3-phosphoinositide-dependent kinase-1 inhibitor.

The molecular complexity of HSV entry has impeded the development of antivirals targeting this process. HSV has been shown to enter most human epithelial cells, including keratinocytes, through a complex calcium-dependent signaling pathway that results in the translocation of Akt to the outer leaflet of the plasma membrane (N. Cheshenko, et al., PLoS Pathog., 14, e1006766 (2018); N. Cheshenko et al., J Cell Biol., 163, 283-293 (2003); and N. Cheshenko, W. et al., Molecular Biology of the Cell, 18, 3119-3130 (2007)). Specifically, binding of HSV envelope glycoproteins C (gC) (HSV-1) or B (gB) (HSV-2) to cellular heparan sulfate proteoglycans and engagement between glycoprotein D (gD) and a cellular receptor, most commonly nectin-1, triggers intracellular calcium ion (Ca²⁺) transients near the plasma membrane. These transients activate phospholipid scramblase 1 (PLSCR1), a Ca²⁺-responsive enzyme responsible for the bidirectional translocation of phospholipids, including phosphatidylserine (PtdS), between the inner and outer leaflets of the plasma membrane. Notably, these lipid movements are associated with the translocation of the Akt kinase to the outer leaflet of the plasma membrane and its subsequent phosphorylation by yet to be determined kinases. Extracellular phosphorylation of Akt is associated with downstream signaling events, perhaps involving outside-in signaling, which culminates in HSV entry and subsequent transport of viral capsids to the nuclear pore (N. Cheshenko et al., J Cell Biol 163, 283-293 (2003); N. Cheshenko, et al, Molecular Biology of the Cell, 18, 3119-3130 (2007); N. Cheshenko et al., FASEB J 27, 2584-2599 (2013); and N. Cheshenko et al., J Virol., 88, 10026-10038 (2014)). Transfection with small interfering RNA (siRNA) targeting PLSCR1 or Akt, or treatment with pharmacological kinase inhibitors prevent HSV entry and infection (N. Cheshenko, et al, PLoS Pathog., 14, e1006766 (2018); N. Cheshenko, W. et al, Molecular Biology of the Cell, 18, 3119-3130 (2007)). It is notable that prolonged PtdS exposure on the outer leaflet of the plasma membrane is an apoptotic signal, which would be detrimental to viral infection and propagation (J. G. Kay, et al, Advances in Experimental Medicine and Biology, 991, 177-193 (2013)). HSV circumvents this liability by orchestrating the translocation of PtdS back to the inner leaflet within 4 hours of viral exposure via a process that is also dependent on PLSCR1 and viral glycoprotein L (gL) (N. Cheshenko, et al, PLoS Pathog., 14, e1006766 (2018)).

Translocation of PtdS to the outer leaflet of the plasma membrane is a well-described phenomenon in cell biology, but the observation that the protein kinase Akt, which typically shuttles between the cytosol and the inner leaflet of the plasma membrane, also becomes accessible on the outer leaflet had not been previously appreciated. This translocation of Akt to the outer leaflet of the plasma membrane may be employed by other viruses and biological processes associated with scramblase activation, as evidenced by findings that ionomycin, a calcium ionophore, also activates PLSCR1 and is associated with externalization and subsequent phosphorylation of Akt (N. Cheshenko, et al, PLoS Pathog., 14, e1006766 (2018)). These observations suggested the possibility that cell-impermeable inhibitors that specifically target extracellular kinase activities could be developed as novel anti-viral drugs and tool compounds to study outside-inside signaling. As a proof of concept, the inventors modified staurosporine, a non-specific ATP-competitive pan-kinase inhibitor that blocks Akt phosphorylation and also triggers cellular apoptosis, to generate a novel cell-impermeable analog. This cell-impermeable staurosporine analog (CIMSS) prevented HSV-induced phosphorylation of Akt at the outer leaflet of the plasma membrane and blocked subsequent HSV entry without inducing apoptosis or inhibiting intracellular Akt phosphorylation (e.g., in response to insulin). Antibodies to Akt also inhibited HSV entry. CIMSS also blocked the extracellular phosphorylation of 3-phosphoinositide dependent protein kinase 1 (PDPK1), and phospholipase C gamma (PLCγ), which were also detected at the outer leaflet of the plasma membrane in response to HSV infection.

Entry of HSV was compared to vesicular stomatitis virus (VSV), an enveloped RNA virus that enters cells by endocytosis. VSV has been used as a vector platform for vaccine development including vaccines for Ebola virus and HIV. Results demonstrate that VSV does not activate the Ca2+-scramblase signaling pathway. VSV infection is not inhibited by drugs that block phospholipid scramblase-1 and its entry is not inhibited by CIMSS.

The spike protein of SARS-CoV-2 binds to the ACE2 receptor to trigger viral entry either via endocytosis or direct fusion, although the relative contribution of each pathway is controversial. For example, recent studies using a lentivirus pseudotyped with SARS-CoV-2 spike protein demonstrated that the virus undergoes rapid, clathrin-mediated endocytosis. (Bayati, A., et al., J Biol Chem, 100306 (2021) doi:10.1016/j.jbc.2021.100306) Another study compared mechanisms of entry for a panel of VSV pseudotyped viruses containing the glycoproteins from Lassa, Ebola, Chikungunya and SARS-CoV-2 with a luciferase reporter. (Lay Mendoza, M. F., et al., Viruses, 12 (2020) doi:10.3390/v12121457) They confirmed that SARS-CoV-2 enters through late endosomes/endolysosomes and requires proteolytic glycoprotein processing. Entry was blocked by ammonium chloride, a lysomotropic agent that prevents endosomal acidification. Based on these observations, it was predicted that CIMSS would have no impact on entry of VSV pseudotyped with the SARS-CoV-2 spike protein (VSV-S). Surprisingly, it was discovered that vesicular stomatitis virus (VSV) pseudotyped with SARS-CoV-2 spike protein (VSV-S), but not native VSV or VSV-pseudotyped with the Ebola viral glycoprotein (VSV-EBOV GP), triggered translocation to, and phosphorylation of Akt, PDPK1, and PLCγ at the outer leaflet of the plasma membrane, and that Akt phosphorylation and VSV-S infection were inhibited by CIMSS. In additional mechanistic studies, it has been demonstrated that VSV-S activates PLSCR to trigger externalization of Akt and other kinases to the outer membrane. In addition, small interfering RNAs (siRNA) targeting PI3K or Akt block VSV-S entry. The inventors also discovered that CIMSS inhibited VSV-S quite efficiently. It was subsequently shown that CIMSS also inhibits native SARS-CoV-2 and HSV virus infection. Polyclonal or monoclonal antibodies targeting Akt or PDPK1 also inhibited VSV-S and HSV-1 and HSV-2 infection. Together these findings suggest a completely novel strategy for inhibiting and/or treating COVID-19 and HSV.

Compounds are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced items. The term “or” means “and/or”. The terms “comprising,” “having,” “including,” and “containing” are to be construed as open-ended terms (i.e., meaning “including, but not limited to”). Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure. Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim.

The opened ended term “comprising” includes the intermediate and closed terms “consisting essentially of” and “consisting of.” Wherever an open ended aspect that may contain additional elements is contemplated (comprising language), more narrow aspects that contain only the listed items (consisting of language) are also contemplated.

A “cell-impermeable inhibitor” as used herein is a compound that inhibits, reduces or blocks the activity of a cellular enzyme or protein and/or decreases the level of the enzyme and/or protein produced by the cell, and which is unable to pass through the plasma membrane and enter the cytosol of the cell.

The terms “plasma membrane” and “cell membrane” are used interchangeably herein and refer to the outer membrane of the cell that regulates the substances entering and exiting the cell.

“Pharmaceutical composition” means a composition comprising at least one active agent, such as a cell permeable inhibitor, and at least one other substance, such as an excipient. An excipient can be a carrier, filler, diluent, bulking agent or other inactive or inert ingredients. Pharmaceutical compositions optionally contain one or more additional active agents. Pharmaceutical compositions meet the U.S. FDA's GMP (good manufacturing practice) standards for human or non-human drugs.

“Pharmaceutically-acceptable carrier” refers to a diluent, adjuvant, excipient, or carrier, other ingredient, or combination of ingredients that alone or together provide a carrier or vehicle with which a compound or compounds of the invention is formulated and/or administered, and in which every ingredient or the carrier as a whole is pharmaceutically acceptable. The pharmaceutically-acceptable carrier includes a carrier that is acceptable for veterinary use as well as human pharmaceutical use. Also included are any and all solvents, dispersion media, coatings, antibacterial and antifungal agents, and isotonic and absorption delaying agents. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. A “pharmaceutically acceptable carrier” includes both one and more than one such carrier.

A “subject” means a human or non-human animal in need of medical treatment. Medical treatment can include treatment of an existing condition, such as a disease or disorder or diagnostic treatment. In an aspect, the patient or the subject is a human patient or human subject. In an aspect the patient or subject is a domesticated companion animal such as a dog or cat.

“Providing” means giving, administering, selling, distributing, transferring (for profit or not), manufacturing, compounding, or dispensing.

“Administering” means giving, providing, applying, or dispensing by any suitable route. Administration of a combination of active agents includes administration of the combination in a single formulation or unit dosage form, administration of the individual active agents of the combination concurrently but separately, or administration of the individual active agents of the combination sequentially by any suitable route. The dosage of the individual active agents of the combination may require more frequent administration of one of the active agent(s) as compared to the other active agent(s) in the combination. Therefore, to permit appropriate dosing, packaged pharmaceutical products may contain one or more dosage forms that contain the combination of active agents, and one or more dosage forms that contain one of the combinations of active agents, but not the other active agent(s) of the combination.

“Treatment” or “treating” means providing the active agent(s) disclosed herein as either the only active agent or together with an additional active agent sufficient to: (a) inhibit the disease, i.e. arrest its development; and (b) relieve the disease, i.e., causing regression of the disease and in the case of a viral infection to eliminate or reduce the virulence of the infection in the subject.

“Inhibiting” means providing a cell permeable inhibitor disclosed herein as either the only active agent or together with an additional active agent sufficient to measurably reduce the ability of a virus to infect a host cell or measurably reduce symptoms of a disease caused by infection of a host cell with the virus.

“Preventing” means administering an amount of a compound of the disclosure sufficient to significantly reduce the likelihood of a disease from occurring in a subject who may be predisposed to the disease but who does not have it. In the context of viral infection “preventing” includes administering an amount of a cell permeable inhibitor disclosed herein to a subject known to be at enhanced risk of viral infection, such as a health care worker likely to be in contact with infected individuals, a family member of an infected individual, or a person living in or traveling in an area where carriers of the viral infection are common.

An “effective amount” or “therapeutically effective amount” of an active agent or a composition including the active agent means an amount effective, when administered to a subject, to provide a therapeutic benefit. The therapeutic benefit can include an amelioration of symptoms, a decrease in disease progression, or inhibiting the development of the disease. An effective amount can vary depending upon a variety of factors including the age, body weight, general health, sex, diet, time of administration, route of administration, rate of excretion, drug combination, and the severity of the particular disorder for the patient undergoing therapy.

An “active agent” is a compound or biological molecule, such as a naturally occurring or non-naturally occurring protein, peptide, hormone, or antibody that exhibits biological activity, such as inhibiting bacteria growth or reproduction, or potentiates the biological activity of a compound of Formula I or Formula II.

A significant reduction is any detectable negative change that is statistically significant in a standard parametric test of statistical significance such as Student's T-test, where p<0.05.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10% or 5% of the stated value.

An “antibody” “Immunoglobulins”, or “antibodies” are proteins selected from among the globulins, which are formed as a reaction of the host organism to a foreign substance (=antigen) from differentiated B-lymphocytes (plasma cells). They serve to defend specifically against these foreign substances. There are various classes of immunoglobulins: IgA, IgD, IgE, IgG, IgM, IgY, IgW. The terms immunoglobulin and antibody are used interchangeably. As used herein, the term “immunoglobulin” or “antibody” includes a polyclonal, monoclonal, monospecific, bi-specific, multi-specific, a single chain antibody, an antigen-binding fragment of an antibody (e.g., an Fab or F(ab′)₂ fragment), a disulfide-linked Fv, etc. Antibodies can be of any species and include chimeric and humanized antibodies. “Chimeric” antibodies are molecules in which antibody domains or regions are derived from different species. For example the variable region of heavy and light chain can be derived from rat or mouse antibody and the constant regions from a human antibody. In “humanized” antibodies only minimal sequences are derived from a non-human species. Often only the CDR amino acid residues of a human antibody are replaced with the CDR amino acid residues of a non-human species such as mouse, rat, rabbit or llama. Sometimes a few key framework amino acid residues with impact on antigen binding specificity and affinity are also replaced by non-human amino acid residues. Antibodies may be produced through chemical synthesis, via recombinant or transgenic means, via cell (e.g., hybridoma) culture, or by other means.

The term “antibody derived molecules” is used interchangeably with “antibody derived fragments” or “antibody fragments” and refers to polypeptides which contain only part(s) of one or more antibody domain(s) or region(s) and/or complete domain(s) or region(s). The antibody fragments can be either a) forming a molecule on their own, b) linked with each other in different combinations, c) fused to non-antibody sequences, d) fused or linked to non-polypeptide (e.g. radionucleotides) or d) any combination of the above. These polypeptides can exist either as monomers or as multimers whereby polypeptides can have identical or different sequences.

“Fab fragments” (Fragment antigen-binding=Fab) or “Fab” consist of the variable regions of both antibody heavy and light chains (VH and VL) which are held together by the adjacent constant regions (CH1 and CL). These may be formed by protease digestion, e.g. with papain, from conventional antibodies, but similar Fab fragments may also be produced in the mean time by genetic engineering. Further antibody fragments include “F(ab′)2 fragments” or “F(ab′)2”, which may be prepared by proteolytic cleaving with pepsin or by genetic engineering in which both Fab arms of an antibody are still linked via inter-heavy chain disulfide bridges located within the hinge region.

The immunoglobulin fragments composed of the CH2 and CH3 domains of the antibody heavy chain are called “Fc fragments”, “Fc region” or “Fc” because of their crystallization propensity (Fc=fragment crystallizable). These may be formed by protease digestion, e.g. with papain or pepsin from conventional antibodies but may also be produced by genetic engineering. The N-terminal part of the Fc fragment might vary depending on how many amino acids of the hinge region are still present.

The term “Fc-fusion protein” describes polypeptides which contain as a fusion partner a natural or modified (e.g. substitutions, deletions, insertions) Fc region of an immunoglobulin. Fc fusion proteins can be either naturally occurring proteins (e.g. antibodies) or engineered recombinant proteins (e.g. TNF receptor-Fc fusion protein or a VH region fused to an Fc region). The Fc-fusion proteins can exist either as monomers or as multimers whereby polypeptides can have identical or different sequences, might contain linker sequences between the two fusion partners and/or part of the hinge region or modified hinge regions or the polypeptide is fused directly to the CH2 domain.

Using genetic engineering methods it is possible to produce shortened antibody fragments which consist only of the variable regions of the heavy (VH) and of the light chain (VL). These are referred to as “Fv fragments” (Fragment variable=fragment of the variable part) or “Fv”. Since these Fv-fragments lack the covalent bonding of the two chains by the cysteines of the constant chains, the Fv fragments are often stabilised. It is advantageous to link the variable regions of the heavy and of the light chain by a short peptide fragment, e.g. of 10 to 30 amino acids, preferably 15 amino acids. In this way a single peptide strand is obtained consisting of VH and VL, linked by a peptide linker. An antibody protein of this kind is known as a “single-chain-Fv” or “scFv”. Examples of scFv-antibody proteins of this kind are known from the prior art. In addition, more than one VH and/or VL region can be linked together. In addition, the polypeptides may multimerise and form homo- or heteromers.

The present disclosure provides a method of preventing or treating a coronavirus infection in a subject or preventing or treating a disease caused by a coronavirus infection in a subject, comprising administering to the subject an effective amount of a cell-impermeable inhibitor comprising a cell-impermeable kinase inhibitor, a cell-impermeable Akt inhibitor, a cell-impermeable 3-phosphoinositide-dependent protein kinase-1 inhibitor, or a combination thereof.

Disclosed herein also is a method of disrupting or reducing infection of a cell by a coronavirus, comprising contacting the cell with an effective amount of a cell-impermeable inhibitor comprising a cell-impermeable kinase inhibitor, a cell-impermeable Akt inhibitor, a cell-impermeable 3-phosphoinositide-dependent protein kinase-1 inhibitor, or a combination thereof.

In an aspect, the coronavirus is severe acute respiratory syndrome coronavirus (SARS Co-V), SARS-CoV-2, or Middle East respiratory syndrome coronavirus (MERS-CoV). The subject can have or be at risk of a disease caused by a coronavirus infection. In an aspect, the disease caused by the coronavirus infection comprises severe acute respiratory syndrome (SARS) or Middle East respiratory syndrome (MERS). In an aspect, the disease caused by the coronavirus infection comprises COVID-19.

Also disclosed are methods of preventing or treating a condition, a disorder or a disease associated with a SARS-CoV-2 infection in a subject comprising administering to the subject an effective amount of a cell-impermeable inhibitor comprising a cell-impermeable kinase inhibitor, a cell-impermeable Akt inhibitor, a cell-impermeable 3-phosphoinositide-dependent protein kinase-1 inhibitor, or a combination thereof.

The present disclosure also provides a method of preventing or treating a herpes simplex virus (HSV) infection in a subject or preventing or treating a disease caused by a herpes simplex virus infection in a subject comprising administering to the subject an effective amount of a cell-impermeable inhibitor comprising a cell-impermeable kinase inhibitor, a cell-impermeable Akt inhibitor, a cell-impermeable 3-phosphoinositide-dependent protein kinase-1 inhibitor, or a combination thereof. In an aspect, the cell-impermeable inhibitor is a cell-impermeable 3-phosphoinositide-dependent protein kinase-1 inhibitor. In an aspect, the HSV comprises HSV-1, HSV-2 or a combination thereof.

In an aspect, the disease caused by the herpes simplex virus infection comprises herpes, oral herpes, herpes whitlow, genital herpes, eczema herpeticum, herpes gladiatorum, HSV keratitis, HSV retinitis, HSV encephalitis or HSV meningitis.

The methods disclosed herein include the administration of a cell impermeable inhibitor to a subject. The cell-impermeable inhibitor inhibits, reduces or blocks the activity of a cellular enzyme or protein involved in or associated with the entry of a virus into a cell. The cell-impermeable inhibitor can be a compound having a structure which is intrinsically impermeable to the plasma membrane or can be a cell-permeable compound which has been structurally modified to be impermeable, i.e., modified to prevent the compound from passing through the cell membrane.

In an aspect, the cell-impermeable inhibitor is a cell-permeable compound (inhibitor) which has been structurally modified to be cell impermeable, and which retains substantially the same level of activity in vitro relative to the unmodified cell-permeable compound. The structural modification can include the covalent linkage of a cell-impermeable moiety to the cell-permeable compound. As used herein a “cell-impermeable moiety” refers to a structure or group, which when covalently linked to the compound, effectively prevents the compound from crossing the plasma membrane of the cell (also referred to herein as the cell membrane).

The cell membrane is impermeable against larger, uncharged polar molecules and all charged molecules including ions. The structure and type of the cell-impermeable moiety is therefore not limited as long as it prevents the modified compound from passing across the cell membrane and does not interfere with the activity of the compound. The cell-impermeable moiety is thus a group which increases polarity/hydrophilicity, hydrogen bonding, and/or size of the compound and/or is a group which decreases the lipophilicity and/or hydrophobicity of the compound. The cell-impermeable moiety can include, for example, a sulfonic acid or sulfonic acid salt, a phosphate, a phosphonate, a phosphoramidate, a phosphodiester, or a salt thereof, a polycarboxylic acid or polycarboxylic acid salt, an amine (e.g., a quaternary amine), a polyamine, a guanidine containing compound, a macromolecule (e.g., albumin, a polymer such as dextran, PEG or a dendrimer, a dye), or a combination thereof. Specific examples include sulfonic acid, sodium sulfonate, sulfoacetic acid, and/or 4-carbonylcyclobutane-1,2,3-tricarboxylic acid.

The cell-impermeable moiety can be connected to the compound directly or via a linker. In an aspect, the cell-impermeable moiety comprises a sodium sulfonate connected to the cell-permeable compound. In an aspect, the sodium sulfonate is connected to the compound via a linker.

In an aspect, the cell-impermeable moiety has the structure of Formula 1

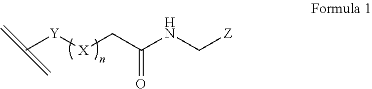

where Y is a bond, carbonyl, or methylene (—CH₂—), X is methylene or polyethylene glycol, n is 0-10, and Z is —C₁-C₈alkylSO₃—, —C₁-C₈alkylSO₄, —C₁-C₈alkylSO₂H, —C₁-C₈alkylPO₄, —C₁-C₈alkylPO(OH)₂, —C₁-C₈alkylPO₃NH₂, or a 5- or 6-membered heterocycle, such as a triazole, substituted with one of —C₁-C₈alkylSO₃—, —C₁-C₈alkylSO₄, —C₁-C₈alkylSO₂H, —C₁-C₈alkylPO₄, —C₁-C₈alkylPO(OH)₂, and —C₁-C₈alkylPO₃NH₂, and optionally substituted with one or more of C₁-C₂alkyl, C₁-C₂alkoxy, halogen, and hydroxyl. Examples of suitable linkers include at least the following linker shown below. The linkers are shown as sodium salts, though other salt forms are possible.

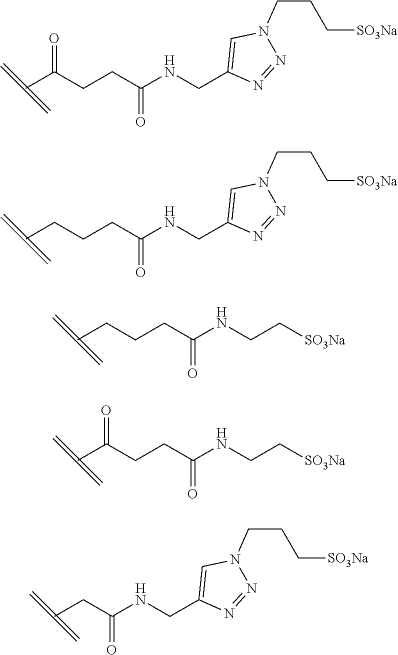

where [symbol] indicates the point of attachment to the inhibitory compound. In an aspect, the linker is attached to the secondary amine of the

As disclosed herein, the cell impermeable inhibitor is an inhibitor of Akt, 3-phosphoinositide-dependent kinase-1 (PDPK1), or a combination thereof. The cell-impermeable inhibitor thus includes a cell-impermeable Akt inhibitor, a cell-impermeable 3-phosphoinositide-dependent kinase-1 inhibitor, or a combination thereof.

Akt, also known as protein kinase B (PKB), is a serine/threonine-specific protein kinase which acts as mediator via the PI3K/Akt pathway. There are three different genes (AKT1, AKT2, and AKT3) encoding isoforms of Akt. The Akt inhibitor can be a small molecule or a large molecule (e.g., an anti-Akt antibody). Non-limiting examples of Akt inhibitors include 3-aminopyrrolidine, anilinotriazole derivatives, 7-azaindole, afuresertib (GSK2110183), A-674563, A-443654, AT7867, AT13148, AZD5363, capivasertib, CCT128930, GDC-006, GSK690693 (aminofurazan), ipatasertib, 6-phenylpurine derivatives, pyrrolo[2,3-d]pyrimidine derivatives, CCT128930, miltefosine, phenylpyrazole derivatives, spiroindoline derivatives, staurosporine, pyrimidyl-5-amidothiophene derivative (DC120), uprosertib (GSK2141795), triazolo[3,4-A][1,6]naphthyridin-3(2H)-one derivative (MK-2206), edelfosine (1-O-octadecyl-2-O-methyl-rac-glycero-3-phosphocholine, ET-18-OCH₃), ilmofosine (BM 41.440), miltefosine (hexadecylphosphocholine, HePC), perifosine (D-21266), erucylphosphocholine (ErPC), erufosine (ErPC3, erucylphosphohomocholine, ndole-3-carbinol, 3-chloroacetylindole, diindolylmethane, diethyl 6-methoxy-5,7-dihydroindolo [2,3-b]carbazole-2,10-dicarboxylate (SR13668), OSU-A9, PH-316, PHT-427, PIT-1, PIT-2, DM-PIT-1, N-[(1-methyl-1H-pyrazol-4-yl)carbonyl]-N′-(3-bromophenyl)-thiourea, triciribine (TCN, NSC 154020), triciribine mono-phosphate active analogue (TCN-P), 4-amino-pyrido[2,3-d]pyrimidine derivative API-1, 3-phenyl-3H-imidazo[4,5-b]pyridine derivatives, ARQ 092, BAY 1125976, 3-methyl-xanthine, quinoline-4-carboxamide and 2-[4-(cyclohexa-1,3-dien-1-yl)-1H-pyrazol-3-yl]phenol, 3-oxo-tirucallic acid, 3α- and 3β-acetoxy-tirucallic acids, acetoxy-tirucallic acid, lactoquinomycin, frenolicin B, kalafungin, medermycin, boc-phe-vinyl ketone, isoquinoline-5-sulfonamides (e.g., H-8, H-89, NL-71-101), 4-hydroxynonenal (4-HNE), 1,6-naphthyridinone derivatives, and imidazo-1,2-pyridine derivatives.

3-phosphoinositide-dependent kinase-1 inhibitor (PDPK1 or PDK1) is a serine/threonine kinase integral to the function of the P13-K/Akt signaling pathway. PDPK1 phosphorylates and activates PKB/Akt and also phosphorylates several members of the AGC (automatic gain control) family of protein kinases. The PDPK1 inhibitor can be a small molecule or a large molecule (e.g., an anti-PDPK1 antibody). Non-limiting examples of PDPK1 inhibitors include BX-795, BX-912, BX-320, BAG-956, 2334470, 2-O-benzyl-myo-inositol 1,3,4,5,6-pentakisphosphate (2-O-Bn-InsP5), OSU-03012 (AR-12), PH-427, and PDK1 inhibitor 2610.

Phospholipid scramblase, or scramblase, is an enzyme present in the cell membrane responsible for Ca²⁺-dependent, non-specific and bidirectional movement (scrambling) of phospholipids from the inner-leaflet to the outer-leaflet of the plasma membrane. In humans, phospholipid scramblases (PLSCRs) constitute a family of five homologous proteins that are named as PLSCR1-PLSCR5. The PLSCR1 inhibitor can be a small molecule inhibitor or a large molecule inhibitor (e.g., antibody, peptide sequence). Non-limiting examples of PLSCR1 include, R5421 (ethanimidothioic acid N-[{5N-butylthio-N-methylamino]-carbonyloxy}-methyl ester, imatinib, monoclonal antibodies (e.g., NP1 (Fan et al, Journal of Translational Medicine volume 10, Article number: 254 (2012)), and a peptide sequence that is a competitive inhibitor of PLSCR1 activity.

In an aspect, the cell impermeable inhibitor is an analog of the Akt inhibitor staurosporine. Staurosporine, with the chemical name (9S,10R,11R,13R)-2,3,10,11,12,13-hexahydro-10-methoxy-9-methyl-11-(methylamino)-9,13-epoxy-1H,9H-diindolo[1,2,3-gh:3′,2′,1′-lm]pyrrolo[3,4-j][1,7]benzodiazonin-1-one, is a cell-permeable, reversible, pan-kinase-ATP-competitive inhibitor of Akt having the following structure:

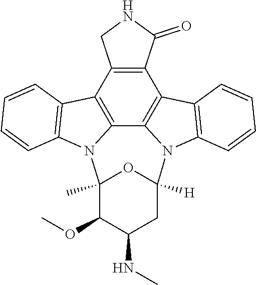 [SMILES: CN[C@@H]1C[C@H]2O[C@@](C)([C@@H]1OC)n1c3ccccc3c3c4c(c5c6ccccc6n2c5c31)C(=O)NC4]

A cell-impermeable moiety is covalently linked to staurosporine to create a cell-impermeable staurosporine analog. In an aspect, the cell-impermeable moiety has the structure of Formula 1 above. In an aspect, the cell-impermeable linker is attached to the secondary amine of staurosporine. In an aspect, the cell-impermeable staurosporine (CIMSS) has the structure of Formula 2, or a pharmaceutically acceptable salt or solvate thereof:

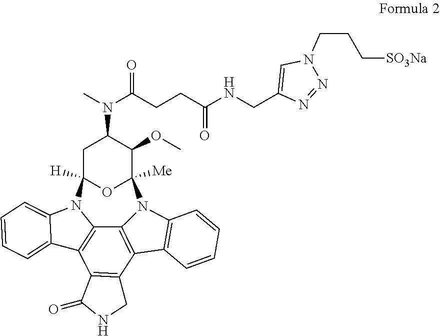

A pharmaceutically acceptable salt includes salts that retain the biological effectiveness and properties of the compound, and which are not biologically or otherwise undesirable, and include derivatives of the disclosed compounds in which the parent compound is modified by making inorganic and organic, non-toxic, acid or base addition salts thereof. The salts can be synthesized from the parent compound by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media such as ether, ethyl acetate, ethanol, isopropanol, or acetonitrile are used, where practicable.

Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the CIMSS parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ-COOH where n is 0-4, and the like. Lists of additional suitable salts may be found, e.g., in G. Steffen Paulekuhn, et al., Journal of Medicinal Chemistry 2007, 50, 6665 and Handbook of Pharmaceutically Acceptable Salts: Properties, Selection and Use, P. Heinrich Stahl and Camille G. Wermuth, Editors, Wiley-VCH, 2002.

A salt of the CIMSS further includes solvates of the compound and of the compound salts. A “solvate” means CIMSS or its pharmaceutically acceptable salt, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions. In an aspect, the solvate is a hydrate

The cell-impermeable inhibitors disclosed herein can be administered alone or in the form of a pharmaceutical composition including the cell-impermeable inhibitor and a pharmaceutically acceptable carrier. Accordingly, the disclosure provides pharmaceutical compositions comprising a cell-impermeable inhibitor and a pharmaceutically acceptable carrier. The pharmaceutical composition can be administered to a subject using any known route of administration. Routes of administration include, but are not limited to, oral, topical, parenteral, intravenous, cutaneous, subcutaneous, intramuscular, inhalation or spray, sublingual, transdermal, intravenous, intrathecal, buccal, nasal, vaginal, rectal, or a combination thereof. In an aspect, the administration of the cell-impermeable inhibitor is oral or parenteral.

The cell-impermeable inhibitor, or a pharmaceutical composition including the cell-impermeable inhibitor is formulated for administration to the subject in a suitable dosage form. The dosage form can be, for example, a capsule, a tablet, an implant, a troche, a lozenge, a minitablet, a suspension, an emulsion, a solution, an aerosol, an injectable, an ovule, a gel, a wafer, a chewable tablet, a powder, a granule, a film, a sprinkle, a pellet, a topical formulation, a patch, a bead, a pill, a powder, a triturate, a smart pill, a smart capsule, a platelet, a strip, or a combination thereof.

The methods of treatment disclosed herein include providing an effective amount of the cell-impermeable inhibitor to a subject. The effective amount can be provided as a dosage form. The effective amount of cell-impermeable inhibitor can be about 0.01 milligrams (mg) to about 10 mg per kilogram of body weight per day. In an aspect, a dosage form including the effective amount of the cell-impermeable inhibitor is provided to the patient. In an aspect the effective amount of cell-impermeable inhibitor is administered to the patient as a single dose or a plurality of doses. For example, the subject can be administered 1 to 4 daily doses. Frequency of dosage may also vary depending on the particular disease treated. However, for treatment of viral infection, a dosage regimen of 4 times daily or less is preferred, and a dosage regimen of 1 or 2 times daily is particularly preferred.

The cell-impermeable inhibitor may be used alone or in combination with an additional active agent (therapeutic agent). Combination use includes administering the cell-impermeable inhibitor and an additional active agent in a single dosage form, or in separate dosage forms, either simultaneously or sequentially. Doses and methods of administration of other therapeutic agents can be found, for example, in the manufacturer's instructions in the Physician's Desk Reference. The cell-impermeable inhibitor may be administered in combination with another active agent for the treatment of coronavirus, herpes simplex virus, or a combination thereof.

The additional active agent for the treatment of coronavirus may include paxlovid, molnupiravir, remdesivir, hydroxychloroquine, chloroquine, ivermectin, clofazimine, a MERS-CoV specific monoclonal antibody (e.g., REGN3048, REG3051), a SARS CoV-2 specific monoclonal antibody (e.g. bamlanivimab, etesevimab, casirivimab, imdevimab, sotrovimab), or a combination thereof.

The additional active agent for the treatment of herpes simplex virus may include acyclovir, famciclovir, valacyclovir, ganciclovir, penciclovir, valaciclovir, Cidofovir, Foscarnet, or a combination thereof.

The compounds and compositions are administered to a subject, and in particular, a subject having a viral infection or at risk of a viral infection. The subject is a mammalian subject. The mammalian subject can be, for example, a human, a rodent, a monkey, a cat, a dog, a bovine animal (cow, steer, bull), a sheep, a monkey, or a primate. In aspects, the mammalian subject is a human. The viral infection is a coronavirus infection and/or a herpes simplex virus infection.

In an aspect, the methods of treating and/or preventing disclosed herein further include administering an effective amount of an additional active agent to the subject, wherein the additional active agent includes insulin, an amylinomimetic drug, or a combination thereof.

In an aspect, the additional active agent includes a short-acting insulin, a rapid-acting insulin, an intermediate-acting insulin, a long-acting insulin, a combination insulin, pramlintide, or a combination thereof.

This disclosure is further illustrated by the following examples, which are non-limiting.

### EXAMPLES

### Materials and Methods

### Synthesis of Cimss

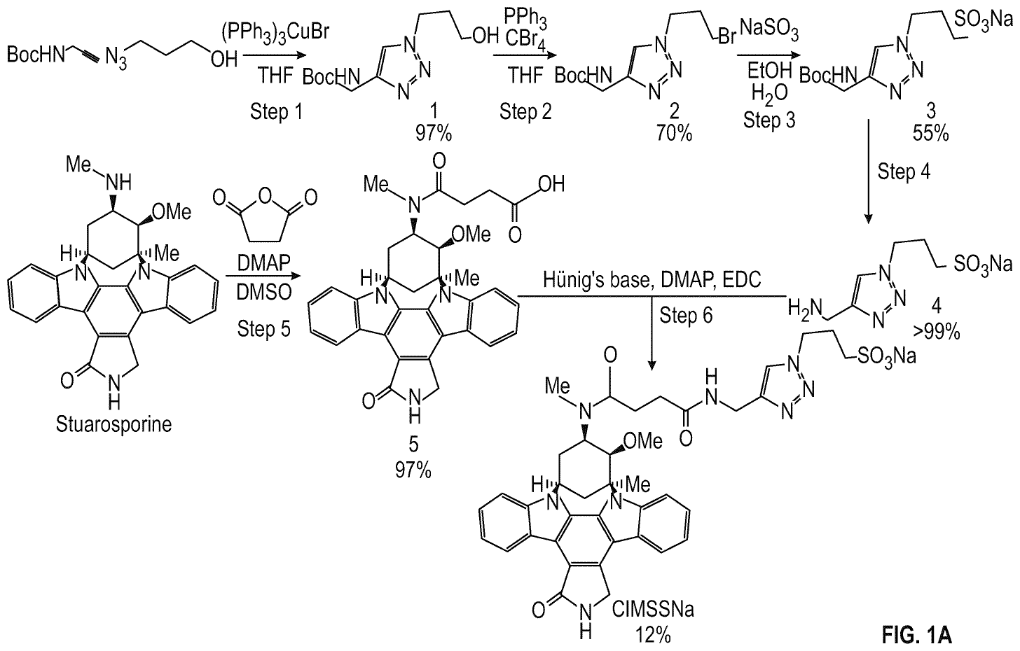

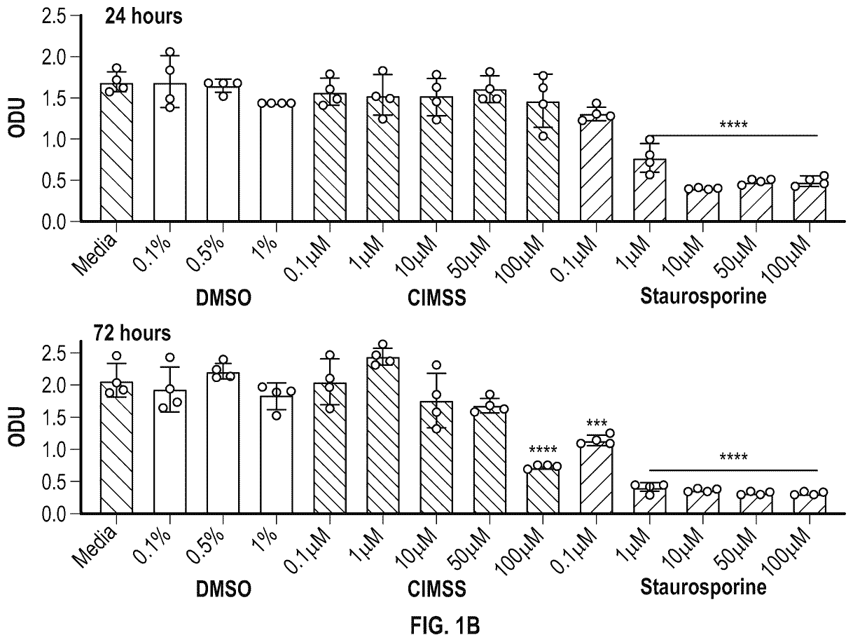

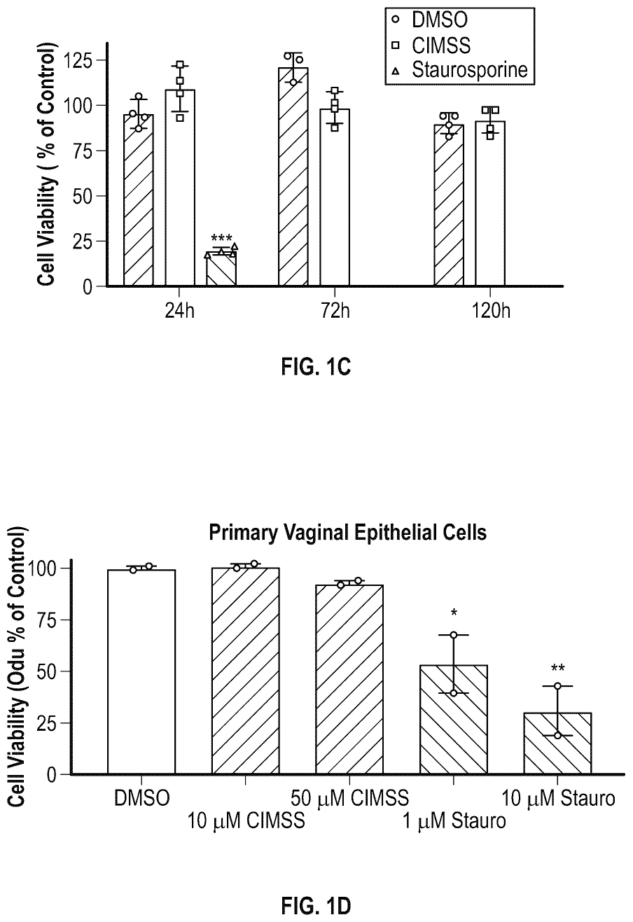

All chemical reagents and solvents were obtained from commercial sources and used without further purification. Microwave reactions were performed using an Anton Paar Monowave 300 reactor. Chromatography was performed on a Teledyne ISCO CombiFlash Rf 200i using disposable silica cartridges. Analytical thin layer chromatography (TLC) was performed on Merck silica gel plates and compounds were visualized using UV or CAM stain. NMR spectra were recorded on a Bruker 600 spectrometer. JH chemical shifts (6) are reported relative to tetramethyl silane (TMS, 0.00 ppm) as internal standard or relative to residual solvent signals. FIG. 1A is a diagram illustrating the synthesis of cell impermeable staurosporine analog (CIMSS).

### STEP 1: TERT-BUTYL ((1-(3-HYDROXYPROPYL)-1H-1,2,3-TRIAZOL-4-YL)METHYL)CARBAMATE (1)

N-Boc-propargylamine (3.1 g, 20 mmol), 3-azidopropan-1-ol (2.0 g, 20 mmol) and THF (50 mL) were combined in a flask which was then purged with Ar. (PPh3)3CuBr (150 mg, 0.16 mmol, 0.8 mol %) (24) was added and the resulting mixture was stirred at room temperature for 5 days. EDTA-Na₄ (300 mg) was added, and the volatiles were removed under reduced pressure. The crude product was loaded on a silica cartridge with CH₂Cl₂ and then purified by flash chromatography (24 g silica, 0-25% Ultra [CH₂Cl₂:MeOH:NH₄OH 75:23:2] in DCM). Minor impurities were removed by a second chromatographic separation (0-100% acetone in hexanes) to give the pure product as a clear oil (4.9 g, 20 mmol, 97%).

TLC: Rf=0.31 (CH₂Cl₂:Ultra 3:1; CAM).

¹H NMR (600 MHz, CHCl₃) δ 7.57 (s, 1H), 5.21 (bs, 1H), 4.51 (t, J 6.8 Hz, 2H), 4.38 (d, J 6.0 Hz, 2H), 3.64 (q, J 5.5 Hz, 2H), 2.34 (t, J 5.1 Hz, 1H), 2.12 (p, J 6.3 Hz, 2H), 1.44 (s, 9H).

¹³C NMR (151 MHz, CDCl₃) δ 155.93, 145.43, 122.43, 79.77, 58.67, 46.92, 36.08, 32.56, 28.39.

ESI-MS: calculated for C₁₁H₂₀N₄O₃(M+H)⁺ 257.1608 found 257.1602.

### STEP 2: TERT-BUTYL ((1-(3-BROMOPROPYL)-1H-1,2,3-TRIAZOL-4-YL)METHYL)CARBAMATE (2)

A flask containing alcohol 1 (2.90 g, 11.3 mmol, 1.0 equiv.) was purged with argon and closed with a septum/Ar balloon. THF (100 mL) was added followed by PPh₃ (5.94 g, 22.6 mmol, 2.0 equiv.) and CBr₄ (7.50 g, 22.6 mmol, 2.0 equiv.). A precipitate formed within 15 minutes. The resulting mixture was stirred at room temperature for 17 hours, before being diluted with Et₂O (50 mL) and filtered. The solids were rinsed with Et₂O (50 mL). Volatiles were removed and the residue purified by column chromatography (24 g silica, 0-40% acetone in hexanes). The product was obtained as an oil that crystalized upon standing (2.53 g, 7.93 mmol, 70%).

TLC: R_{f}=0.29 (hexanes:acetone 1:1; I₂ then CAM).

¹H NMR (600 MHz, CDCl₃) δ 7.57 (s, 1H), 5.11 (bs, 1H), 4.53 (t, J 6.6 Hz, 2H), 4.40 (d, J 6.0 Hz, 2H), 3.36 (t, J 6.2 Hz, 2H), 2.46 (p, J 6.4 Hz, 2H), 1.44 (s, 9H).

¹³C NMR (151 MHz, CDCl₃) δ 155.85, 145.53, 122.49, 79.75, 48.10, 36.09, 32.55, 29.32, 28.37. ESI-MS: calculated for C₁₁H₂₀BrN₄O₂(M+H)⁺ 319.0764 found 319.0766.

### STEP 3: SODIUM 3-(4-(((TERT-BUTOXYCARBONYL)AMINO)METHYL)-1H-1,2,3-TRIAZOL-1-YL)PROPANE-1-SULFONATE (3)

Bromide 2 (530 mg, 1.66 mmol, 1.0 equiv.), ethanol (2 mL), water (1 mL) and sodium sulfite (523 mg, 4.15 mmol, 2.5 equiv.) were added to a microwave vial. The vial was capped and heated to 80° C. for 6 hours. Mass spectrometric analysis showed full conversion of the starting material. The insoluble material was removed by filtration (glasswool) and rinsed with ethanol. The liquid phase was concentrated almost to dryness and the product was precipitated by addition of acetone (5 mL). The solids were collected by filtration and rinsed with acetone and CH₂Cl₂ to give the sulfonate salt in good purity (310 mg, 0.91 mmol, 55%).

¹H NMR (600 MHz, D₂O) δ 7.92 (s, 1H), 4.57 (t, J 6.8 Hz, 2H), 4.34 (s, 2H), 2.87 (t, J7.7 Hz, 2H), 2.34 (p, J 7.0 Hz, 2H), 1.43 (s, 9H).

¹³C NMR (151 MHz, D₂O) 6 181.36, 157.97, 123.52, 81.36, 48.72, 47.61, 30.21, 27.57, 25.15.

ESI-MS: calculated for C₁₁H₂₀N₄O₅Na (M+H)⁺ 343.1047 found 343.1044.

### STEP 4: SODIUM 3-(4-(AMINOMETHYL)-1H-1,2,3-TRIAZOL-1-YL)PROPANE-1-SULFONATE (4)

Boc-protected amine 4 (53 mg, 0.15 mmol) was dissolved in water and heated to 150° C. for 30 min in a microwave vial. The product was obtained as a white powder after freeze-drying (38 mg, 0.15 mmol, >99%).

¹H NMR (600 MHz, D₂0) 6 8.02 (s, 1H), 4.60 (t, J 6.9 Hz, 2H), 4.08 (s, 2H), 2.93-2.80 (m, 2H), 2.36 (p, J 7.0 Hz, 2H).

¹³C NMR (151 MHz, D₂0) 6 145.38, 123.99, 48.74, 47.58, 34.99, 25.11.

ESI-MS: calculated for C₆H₁2N403S (M+H)+243.0523 found 243.0524.

### STEP 5: STAUROSPORINE N-4-OXOBUTANOIC ACID (5) (20, 25)

Staurosporine (16 mg, 34 mmol, 1.0 equiv.), succinic anhydride (10 mg, 0.10 mmol, 2.9 equiv.), 4-dimethylamino pyridine (DMAP; 8.4 mg, 69 mmol, 2.0 equiv.) and dimethyl sulfoxide (DMSO; 1 mL) were combined in a vial, and the resulting solution was stirred overnight while being protected from light. The reaction mixture was diluted with EtOAc (5 mL) and transferred to a Falcon tube containing water (5 mL) and 1 M HCl (1 mL). The phases were separated, and the aqueous layer was extracted with EtOAc (3×2 mL). The combined organic layers were combined, dried (Na₂SO₄), filtered, and concentrated. The residues were loaded on a silica cartridge with CH₂Cl₂ and a minimum of MeOH. The product was obtained as an off-white solid after chromatography (4 g silica, 0-10% MeOH in CH₂Cl₂). Yield: 18 mg, 32 mmol, 93%.

TLC: R_{f}=0.17 (CH₂Cl₂:MeOH 9:1; UV).

¹H NMR (600 MHz, DMSO-d6) δ 9.29 (d, J=7.8 Hz, 1H), 8.59 (s, 1H), 8.15 (d, J=6.3 Hz, 2H), 8.06 (d, J=7.8 Hz, 1H), 7.99 (d, J=8.4 Hz, 1H), 7.67 (d, J=8.2 Hz, 1H), 7.53-7.46 (m, 2H), 7.36 (t, J=7.5 Hz, 1H), 7.30 (t, J=7.5 Hz, 1H), 7.03 (dd, J=8.5, 6.7 Hz, 1H), 6.77 (d, J=7.1 Hz, 2H), 5.00 (s, 3H), 2.82 (s, 3H), 2.77 (s, 3H), 2.68 (d, J=10.9 Hz, 1H), 2.62-2.56 (m, 1H), 2.24 (td, J=13.1, 6.7 Hz, 1H).

### STEP 6: NACIMSS

Acid 5 (18 mg, 32 mmol, 1.0 equiv.) and amine 4 (15 mg, 65 mmol, 2.0 equiv) were dissolved in DMSO (1 mL). Hünig's base (28 mL, 0.16 mmol, 5.0 equiv.), DMAP (4 mg, 32 mmol, 1.0 equiv.) and 1-ethyl-3-(3-dimethylaminopropyl)carbodiimide (EDC; 12 mg, 64 mmol, 2.0 equiv.) were added and the mixture was stirred at room temperature overnight while being protected from light. Thin layer chromatography (Ultra², UV) showed practically complete conversion and the solvent was removed under high vacuum. The crude product was dissolved in methanol and absorbed on silica. NaCIMSS, having the structure shown below, was obtained as a white powder after column chromatography (4 g silica, Pump A: EtOAc:acetone 4:1. Pump B: MeOH:H₂O 1:1; 0-20% B). 3.0 mg, 12%.

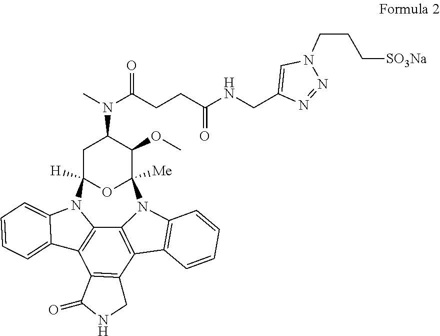

TLC: R_{f}=0.45 (EtOAc:Acetone:MeOH:H₂O 4:1:1:1; UV).

¹H NMR (600 MHz, DMSO-d6) δ 9.29 (d, J=7.8 Hz, 1H), 8.60 (s, 1H), 8.37 (t, J=5.6 Hz, 1H), 8.06 (d, J=7.9 Hz, 1H), 8.01 (d, J=8.5 Hz, 1H), 7.95 (s, 1H), 7.69 (d, J=8.2 Hz, 1H), 7.49 (td, J=7.4, 3.5 Hz, 2H), 7.36 (t, J=7.5 Hz, 1H), 7.30 (t, J=7.4 Hz, 1H), 7.06 (dd, J=8.4, 6.9 Hz, 1H), 5.01 (s, 3H), 4.46 (t, J=7.0 Hz, 3H), 4.32 (d, J=5.6 Hz, 2H), 4.24 (s, 1H), 2.83 (s, 3H), 2.78 (s, 3H), 2.72-2.58 (m, 4H), 2.47-2.37 (m, 5H), 2.34 (s, 3H), 2.22 (td, J=13.0, 6.7 Hz, 1H), 2.13-2.06 (m, 2H).

¹³C NMR (151 MHz, DMSO-d6) δ 172.62, 172.36, 171.81, 145.36, 139.36, 136.73, 133.12, 126.11, 125.78, 125.46, 124.20, 123.37, 123.07, 121.88, 120.74, 119.89, 119.83, 115.65, 114.56, 114.17, 109.51, 95.13, 83.70, 82.72, 60.96, 55.39, 48.97, 48.57, 48.51, 45.88, 34.79, 31.28, 30.58, 29.96, 29.05, 27.24, 27.07, 27.05.

ESI-MS: calculated for C₃₈H₄₀N₈O₈S (M+H)⁺ 791.2582 found 791.2513.

### In Vitro Kinase Assays and IC50 Studies

In vitro kinase assays studies: Kinase activity and IC₅₀ studies were performed by Reaction Biology Corporation using the “HotSpot” assay platform modified from the published procedure (26). Briefly, into a base reaction buffer (20 mM Hepes (pH 7.5), 10 mM MgCl₂, 1 mM EGTA, 0.01% Brij35, 0.02 mg/mL BSA, 0.1 mM Na₃VO₄, 2 mM DTT, 1% DMSO) containing substrate were delivered sequentially 1) required cofactors, 2) kinase enzyme (followed by gentle mixing), 3) compound (CIMSS or controls) in 100% DMSO (introduced by acoustic technology (Echo550; nanoliter range, followed by incubation at 20° C. for 20 minutes), and finally 4) ³³P-ATP to initiate the reaction. After two hours, kinase activity was detected by the P81 filter-binding method. The concentration of drug that inhibited 50% of kinase activity (IC₅₀) was calculated relative to controls.

### Cell Permeability Assay

MDCK Transport analysis was performed by Quintara Discovery (Hayward, CA). MDCK-MDR1 cell plates were maintained for 3 days at 37° C. with 5% CO₂. Cells were washed with Hank's Balanced Salt Solution (HBSS) with 5 mM HEPES for 30 min before starting the experiment. Test compound (CIMSS and controls digoxin and propranolol) solutions were prepared by diluting from DMSO stock into HBSS buffer to a final concentration of 5 μM. Prior to the experiment, cell monolayer integrity was verified by transendothelial electrical resistance (TEER). All of the wells had high resistance above the acceptance cut-off (1 kΩ). The test compounds were added to the apical (75 μL) side with blank buffer on the basal side (250 μL). A P-glycoprotein (gp) inhibitor (GF120918, 10 μM) was maintained in the transport buffer to block the active P-gp transporter. Transport plates were incubated at 37° C. in a humidified incubator with 5% CO₂. A sample was obtained from the donor compartment at time zero and from donor and acceptor compartments after 1 hour and analyzed by liquid chromatography with tandem mass spectrometry (LC/MS/MS). Apparent permeability (Papp) values were calculated using the equation: Papp=(dQ/dt)/A/CO where dQ/dt is the initial rate of amount of test compound transported across cell monolayer, A is the surface area of the filter membrane, and CO is the concentration of the test compound at time zero.

PAMPA analysis was performed by Quintara Discovery (Hayward, CA). Pre-coated 96-well PAMPA plate system was purchased from Corning and left to thaw to the room temperature for at least 30 minutes before use. Compounds (CIMSS and controls atenolol and propranolol) were dissolved at 100 μM in PBS, pH 7.4 with a final DMSO concentration of 1%. The transport experiment was initiated by adding test compounds to the donor (300 μL) side and with blank buffer on the receiver side (200 μL) side. The transport plate was incubated at the room temperature for 5 hours. Samples were obtained from the donor compartment at time zero and from the donor and acceptor compartments after 5 hour and analyzed by liquid chromatography with tandem mass spectrometry (LC/MS/MS).

### Cells and Viruses

Vero (monkey kidney epithelial cell line CCL-81) (American Type Culture Collection (ATTC), Manassas, VA), HaCat (human keratinocyte spontaneously immortalized cell line; ATTC CLS 300493), Vero E6 (ATCC CRL-1586), Calu-3 cells (human lung adenocarcinoma epithelial cells, ATCC)and the human hepatocyte-derived cellular carcinoma Huh7 cell line and its derivative Huh7.5 were maintained in Dulbecco's modified Eagle medium (DMEM, ThermoFisher, USA) supplemented with 10% fetal bovine serum (FBS, ThermoFisher, USA) and with 1% nonessential amino acids (Caco-2 and Huh-7.5 cells) 20 units/mL penicillin combined with 20 μg/mL streptomycin (Sigma, USA), or with 10% fetal bovine serum, 90 μg/ml streptomycine, 40 μg/ml penicillin, 44 mM sodium bicarbonate and 1 mM sodium pyruvate (Calu-3). Cells were cultured in 5% CO₂ at 37° C. Primary human vaginal epithelial cells (ATCC PCS-480-010) were cultured in epithelial mammary cell basal media supplemented with 5 μg/ml insulin, 0.004 ml/ml bovine pituitary extract, 10 ng/ml epidermal growth factor and 0.5 μg/ml hydrocortisone (PromoCell. GmbH, Germany).

The HSV strains included HSV-1(KOS), HSV-2(G), HSV-1 (KVP26GFP), which contains a green fluorescent protein (GFP) fused to the viral capsid proteinVP26 (GFP-VP26 fusion protein) (Desai et al., 1998, J Virol 72, 7563-7568), HSV-1 (F-GS2822), which expresses a red fluorescent protein fused to the N terminus of the capsid protein VP26 (gift from Gregory Smith, Northwestern University) (Antinone and Smith, 2010, J Virol 84, 1504-1512), and HSV-2(333ZAG), which expresses GFP under the control of a cytomegalovirus (CMV) promoter inserted in an intergenic region(Nixon et al., 2013, J Virol 87, 6257-6269). The VSV viruses included native VSV (Indiana) and VSV viruses engineered to express enhanced GFP and either VSV glycoprotein G (VSV-G), Ebola virus glycoprotein (VSV-EBOV-GP) or SARS-CoV-2 spike (VSV-S) and were provided by K. Chandran, Albert Einstein College of Medicine) and propagated and titered on Vero cells (Mulherkar et al., 2011, Virology 419, 72-83; Dieterle et al., 2020, Cell Host Microbe 28, 486-496). SARS-CoV-2 (strain USA-WA1/2020) was obtained from BEI Resources and propagated on Caco-2 cells. Caco-2 cells were infected at a MOI=0.05 PFU/cell and incubated for 6 days at 37° C. The virus-containing supernatant was subsequently harvested, clarified by centrifugation (3,000 g×10 min) and stored at −80° C. Viral titers were measured on Huh-7.5 cells by standard plaque assay. Briefly, 500 μL of serial 10-fold virus dilutions in Opti-MEM™ were used to infect 4×10⁵ cells seeded the day prior into wells of a 6-well plate. After 90 min adsorption, the virus inoculum was removed, and cells were overlayed with DMEM containing 10% FBS with 1.2% microcrystalline cellulose (Avicel®). Cells were incubated for 4 days at 33° C., followed by fixation with 7% formaldehyde and crystal violet staining for plaque enumeration.

### Cytotoxicity and Apoptosis Assays

To assess effects of CIMSS on cell growth and viability, Vero or HaCat cells were plated in 96-well plates and allowed to adhere overnight to ˜50% confluence (growth) or to 90-100% confluence (cytotoxicity) and then exposed to culture media alone, media containing increasing concentrations CIMSS (prepared as described above) or staurosporine or an equivalent concentration of DMSO (0.1% DMSO for 10 μM CIMSS or staurosporine, 0.5% for 50 μM and 1% DMSO for 100 μM). Cell proliferation and viability were determined using the Cell Titer 96® Aqueous One Solution and optical density was determined using a SpectraMax® M5e Molecular Devices multidetection microplate reader. For apoptosis assays, HaCat cells were grown on glass coverslips in 24-well plates and then exposed to 0.1% DMSO, CIMSS (10 μM) or staurosporine (10 μM) for 6 and 24 hrs. The cells were then fixed and stained for activated caspases (red) and SYTOX Green (for integrity of plasma membrane) by Image-iT™ LIVE red Poly Caspase Detection kit. Nuclei were stained with Hoechst. Images were acquired by laser confocal microscope ZeissLive/DuoScan equipped with oil immersion objectives 63×1.1 and captured in an optical slice of 0.37 μm with appropriate filters. Alternatively, immunoblots were prepared and probed with antibodies to cleaved PARP-1 or cleaved caspase 8.

### HSV Plaque, Binding, and Entry Assays

For plaque assays, HaCat cells were exposed to the indicated multiplicity of infection (MOI) for 1 h at 37° C. in the presence of DMSO, CIMSS or staurosporine, washed once with low pH buffer to inactivate extracellular virus and then three times with phosphate buffered saline (PBS, pH 7.4), before culturing in serum free media. After 48 h incubation, plaques were counted by immunoassay using an anti-human IgG antibody peroxidase conjugate. (PA1-86064, Thermo Fisher) (B. C. Herold, et al., J Virol 65, 1090-1098 (1991)). To evaluate the effects of drugs on viral binding, HaCat cells were exposed to escalating MOI of virus in the absence or presence of CIMSS for 4 h at 4° C. Western blots were prepared and probed for gD as a marker of bound virus and beta-actin as a control as previously described (N. Cheshenko, et el., J Gen Virol 83, 2247-2255 (2002). To quantify viral entry, cells were synchronously infected by allowing virus to bind to cells at 4° C. for 4h, washed, and then transferred to 37° C. CIMSS or control DMSO was added at the time of temperature shift. At indicated times post-temperature shift, the cells were treated with a low pH buffer to inactivate virus that had not penetrated, washed, overlaid with medium containing methyl cellulose and vial plaques were counted at 48h. Entry was also assessed by immunoblotting nuclear extracts for VP16. HaCat or primary vaginal epithelial cells were mock-infected or infected with HSV-2(G) (MOI 10 pfu/cell) at 4° C. for 60 min, washed 3 times with cold PBS and overlaid with medium supplemented with DMSO (0.1%), CIMSS (10 μM), rabbit-anti-human Akt IgG (2 g/ml) or control IgG (2 μg/ml). Nuclear extracts were prepared after 1 h and analyzed by Western blot with antibodies targeting VP16, histone H1 and golgin-97 as previously described. The non-nuclear fraction was also probed for golgin-97 as a positive control. Additionally, HaCat cells were mock-infected or synchronously infected with HSV-1 K26GFP and, at time of temperature shift, treated with 10 uM CIMSS, 2ug/ml polyclonal anti-Akt antibody, 10 μg/ml cycloheximide or 0.1% DMSO. After 1 or 4 hours of incubation, the cells were fixed. Plasma membranes were stained using Image-IT™ LIVE Plasma Membrane kit and nuclei were stained with DAPI (Thermo Fisher Scientific, Cat 134406). The percentage of GFP+ nuclei was determined by counting about 200 cells over 3-4 fields. Images were obtained with Leica SP8 microscope (objective 63×1.4); xz images were captured with optical slice 0.6 m, 25-30 slices per image.

### VSV Pseudotyped Virus Infection Assays

Infection of Vero or Huh7 cells by VSV-G, VSV-EBOV-GP and VSV-S was monitored by plaque assay after 48 h culture and staining with crystal violet. Calu-3 cells were infected with virus and after 96 h culture, supernatants were harvested and viral yields quantified by titering on Vero cells.

### SARS-COV-2 Assays

The day prior to infection Huh-7.5 cells were seeded into 96-well plates at two densities: 1.25×10⁴ cells/well and at 5×10³ cells/well for fixation at 24 hpi and at 72 hpi, respectively. The next day, serially diluted CIMSS (or DMSO control) was added to the wells, followed by infections with SARS-CoV-2 at MOIs of 0.25 PFU/cell (24 h timepoint) and 0.01 PFU/cell (72 h timepoint). Cells were then incubated at 37° C. for 24 h and at 33° C. for 72 h. At the respective timepoints, cells were fixed by adding an equal volume of 7% formaldehyde to the wells and subsequently permeabilized with 0.1% Triton X-100 for 10 min. After extensive washing, SARS-CoV-2 infected cells were incubated for 1 h at room temperature with blocking solution of 5% goat serum in PBS (catalog no. 005-000-121; Jackson ImmunoResearch). A rabbit polyclonal anti-SARS-CoV-2 nucleocapsid antibody (catalog no. GTX135357; GeneTex) was added to the cells at 1:1000 dilution in blocking solution and incubated at 4° C. overnight. Goat anti-rabbit AlexaFluor® 594 (catalog no. A-11012; Life Technologies™) was used as a secondary antibody at a 1:2000 dilution. Nuclei were stained with Hoechst 33342 (catalog no. 62249; Thermo Scientific) at a 1 μg/mL dilution. Images were acquired with a fluorescence microscope and analyzed using ImageXpress Micro XLS (Molecular Devices, Sunnyvale, CA). All SARS-CoV-2 experiments were performed in a biosafety level 3 laboratory.

### Antibodies and Chemical Reagents

Primary antibodies and dilutions were as follows: mouse anti-PtdS mAb, 1:200 (05-719, Millipore, Upstate® Biotechnology, Lake Placid, NY); mouse anti-PLSCR1 mAb, 1:200 (ab24923, Abcam® Cambridge, MA), rabbit anti-PLSCR1, 1:500 (NBP1-322588, NOVUS Biologicals™, Littleton, CO); mouse anti-PLSCR1 (sc-27779, Santa Cruz Biotechnology®); mouse anti-phosphotyrosine mAb (4G10; 05-1050X, Millipore,); rabbit anti-FIC1, 1:500 (sc-134967, Santa Cruz Biotechnology®), mouse anti-3-actin mAb, 1:5000 (A-5441, Sigma-Aldrich®); rabbit anti-phospho-Akt (Ser-473) mAb, 1:500 (4060T, Cell Signaling Technology®, Danvers, MA); rabbit anti-phospho-Akt (Thr-308) mAb, 1:500 (9275, Cell Signaling Technology®); rabbit anti-Akt123, 1:1000 (sc-8312, Santa Cruz Biotechnology®); rabbit anti-Akt, 1:200 (9272S, Cell Signaling Technology®); rabbit anti-Akt1, 1:200 (SAB450007, Sigma Aldrich®); rabbit anti-pan-Akt (phosphoT308), 1:250 (ab38449, Abcam®); rabbit anti-PDPK1, 1:200 (3062S, Cell Signaling Technology®); rabbit anti-pPDPK1(S241), 1:300 (3438S, Cell Signaling Technology®); rabbit anti-pPLCγ1,1:300 (07-506, Upstate); mouse anti-PLCγ1, 1:200 (sc-374467, Santa Cruz Biotechnology®); rabbit anti-cleaved caspase 8 (Asp374) (18C8), 1:1000 (9496, Cell Signaling Technology®); rabbit anti-cleaved PARP-1 (Asp 214) (D64E10) XP, 1:1000 (5625, Cell Signaling Technology®); mouse anti-ACE2, 1:100 (sc390851, Santa Cruz Biotechnology®), mouse anti-TPMRSS2, 1:100 (sc-51572, Santa Cruz Biotechnology®), human anti-SARS-CoV2 Spike protein, 1:100 (703973, Invitrogen™); mouse anti-HSV gD, 1:100 (HA025, Virusys Corporation, Taneytown, MD); goat anti-HSV VP16, 2 μg/ml (sc-17547, Santa Cruz Biotechnology®); mouse anti-histone H1, 2 μg/ml (sc-8030, Santa Cruz Biotechnology®); mouse anti-golgin97 (sc-59820, Santa Cruz Biotechnology®); rabbit anti-caspase 8 pAb, 1:200 (NB100-56116 NOVUSBIO™), 1:200 (PAS-38388, Invitrogen™); rabbit anti-pPKA C (T197), 1:200 (5661S, Cell Signaling Technology®); anti-goat IgG, 1:500 (STAR AR122, Bio-Rad), mouse IgG, 1:100 (sc-2025, Santa Cruz Biotechnology®, goat anti-p-actin (Thermo Fisher Scientific), and rabbit anti-GFP, 1:250 (ab32146, Abcam®). Annexin V Alexa Fluor® 555 was purchased from Thermo Fisher Scientific (A 35108). The secondary antibodies for Western blots were horseradish peroxidase-conjugated goat anti-mouse (170-5047, Bio-Rad, Hercules, CA), goat anti-rabbit (170-5046, Bio-Rad), and donkey anti-goat 1:1000 (sc-2020, Santa Cruz Biotechnology®). The secondary antibodies for confocal microscopy were goat anti-mouse Alexa Fluor® 350 (A-11045, Invitrogen Molecular Probes™), goat anti-mouse Alexa Fluor® 555 (A-21147, Thermo Fisher), goat anti mouse Alexa Fluor® 488 (Al 1001, Thermo Fisher) and goat anti-rabbit Alexa Fluor® 488 (A-11078, Thermo Fisher) or Alexa Fluor® 555 (A-21428, Thermo Fisher). All secondary antibodies were diluted 1:1000. Staurosporine (PHZ1271) was purchased from Invitrogen Molecular Probes™ and apyrase from New England BioLabs® (M0398S). Cell Titer 96 Aqueous One solution Cell proliferation Assay” was purchased from Promega (G3580 Promega, San Luis Obispo, CA, USA), Image-iT™ LIVE red Poly Caspases Detection kit was purchased from Invitrogen™ (I35101, Thermo Fisher Scientific). Human recombinant insulin was purchased from MP Biomedicals™

### Confocal Microscopy

Cells were grown on glass coverslips in 12- or 24-well plates and treated with 10 M insulin in the absence or presence of CIMSS (10 or 100 μM) or staurosporine (0.1, 1 or 10 μM), fixed with 4% paraformaldehyde solution (Electron Microscopy, Hatfield, PA, USA) with or without permeabilization with 0.1% Triton-X and stained with conjugated antibodies to detect total or phosphorylated Akt (pAkt^{t308}) and with DAPI (D1306, Invitrogen™) to detect nuclei. In other experiments, cells were exposed to indicated viruses in the absence or presence of drug (CIMSS, staurosporine or DMSO control) and at different times post-viral exposure fixed with or without permeabilization. To label plasma membranes cells were incubated with blue-fluorescent Alexa Fluor® 350 wheat germ agglutinin (I34406, W11261, Invitrogen Molecular Probes™ Carlsbad, CA, USA) before infection. Conjugated antibodies to detect other cellular proteins are noted above (Antibodies and chemical reagents). Images were acquired by laser confocal microscope ZeissLive/DuoScan equipped with oil immersion objectives 63×1.4 and 100×1.4 or Leica SP8 equipped with oil immersion objectives 63×1.4, captured in an optical slice of ˜0.37 μm with appropriate filters. Alexa Fluor® 488 and GFP were excited using the 488-nm line of a krypton/argon laser and viewed with a 505- to 530-nm band pass pm; AlexaFluor® 350 was excited with 405-nm diode laser and collected with 420 to 475 nm filter; AlexaFluor® 555 was excited using 561-nm helium/neon laser and collected with a 575 to 655 filter. All images were captured using the multitrack mode of the microscope to decrease cross talk of fluorescent signals (Zeiss LSM); 3D and extended focus images were generated using Volocity 5.3 software (Improvision, Perkin Elmer®, Lexington, MA). Images on Leica Sp8 were captured using excitation lines 405 nm, Argon (458 nm, 476 nm, 488 nm, 496 nm, 524 nm), collected by adjustable emission windows. All images were captured using one HyD, two PMTs (photomultipliers) and processed by LAS X. The number of GFP, PLSCR1, Akt, PtdS, PDPK1 and PLCγ1 positive cells was quantified using Cell Counter ImageJ software (NIH).

### Western Blots

Cells were serum-starved for 24 h and then exposed to HSV-2(G) (MOI 10 pfu/cell) in the presence of control buffer (0.1% DMSO) or CIMSS (10IM). At different times post-viral exposure, the cells were harvested and lysed in buffer containing 20 mM Tris pH 7.5, 50 mM NaCl, 1% NP-40, 0.05% DOC, supplemented with fresh protease and phosphatase inhibitors (118735, Roche Diagnostics, and P0044, P5726, Sigma Aldrich®, respectively). Proteins were separated by SDS-PAGE and transferred to membranes for immunoblotting with the indicated antibodies. Blots were visualized, scanned and the band intensities were analyzed using ChemiDoc imaging system equipped with GelDoc2000 software (RRID:SCR_014210, Bio Rad). Western blots were quantified using ImageJ software (NIH).

### Biotinylation of Cell Surface Proteins

Cells were exposed to HSV-2 (10 pfu/cell) or mock infected in the presence of control buffer (0.1% DMSO) or CIMSS (10 μM) for 15 and 30 minutes, washed 4 times with ice-cold PBS and biotinylated with sulfo-NHS-SS-Biotin (F20650; Invitrogen Molecular Probes™) for 1 hour at 4° C. After 3 washes with PBS supplemented with 1% BSA, cells were harvested, solubilized in PBS containing a proteinase inhibitor cocktail, precipitated with streptavidin magnetic beads (Dynabeads™ M-280 Streptavidin; Life Technologies™ Gaithersburg, MD, USA) and analyzed by immunoblotting.

### Calcium Kinetic Measurements

HaCat cells (5×10⁴) were seeded in 96 well black plates with clear bottoms (3340, CellBIND® surface, Corning® Inc., NY) and incubated with 25 μM Fura-2 AM diluted in PBS (F1221, Invitrogen Molecular Probes™) for 60 min at 37° C., rinsed with PBS thrice, placed on ice and then exposed to cold purified HSV-2 (5 pfu/cell) or control buffer (PBS). The cells were then transferred to SpectraMax® MF^{e} temperature-regulated chamber at 37° C. (Molecular Devices™, Ca) without washing; photometric data for intracellular Ca²⁺ concentration [Ca²⁺] were generated by exciting cells at 340 and 380 nm and measuring emission at 510 nm every minute for one hour using SoftMax® Pro. 5.4 software (Molecular Devices™). An intracellular calibration was performed with each experiment by determining the fluorescence ratio (340:380) in the presence of Ca-free 10 mM K2EGTA buffer (Rₘᵢₙ) and 10 mM CaEGTA buffer containing 10 μM ionomycin (Rₘₐₓ) (C-3008, Calcium Calibration Buffer Kit #1, Invitrogen Molecular Probes™). The mean [Ca²⁺] was determined from four wells according to the manufacturer's recommendations using the following equation: [Ca²⁺]=K_{d}Q (R-Rₘᵢₙ)/(Rₘₐₓ-R), where R represents the fluorescence intensity ratio F_{λ1}/F_{λ2}; λ1(340 nm) and λ2 (380 nm) are the fluorescence detection wavelengths for ion-bound and ion-free indicators; K_{d} is the Ca²⁺ dissociation constant and equals 0.14 μM (Fura and Indo Ratiometric Calcium Indicators, Invitrogen Molecular Probes™); and Q is the ratio of Fₘᵢₙ to Fₘₐₓ at λ2 (380 nm).

### Immunoprecipitation Assays

Cells were serum-starved for 24 h prior to being exposed to HSV-2(G) (10 pfu/cell) in the presence of control buffer (0.1% DMSO) or CIMSS (10 μM for 30 min, washed three times with PBS, and then immediately placed on ice. The cells were then lysed by sonication in RIPA buffer (Thermo Scientific) supplemented with complete protease inhibitors (Roche Diagnostics). The lysates were incubated overnight at 4° C. with rabbit anti-PLSR1 and then immune complexes were isolated following a 4-h incubation with protein G Plus agarose beads (sc-500778, Santa Cruz Biotechnology®). The precipitated complexes (pellet), supernatants or an aliquot of the cell lysate were analyzed by Western blot with mouse anti-scramblase mAbs or with mouse anti-phosphotyrosine mAb (mPY).

Small Interfering RNA (siRNA) Transfections

Cells were transfected with 10 nM of the indicated siRNA sequences in 12-well plates using the HiPerFect Transfection Reagent (1029975, Qiagen). Akt1 siRNA(sc-29195) was purchased from Santa Cruz Biotechnology® (Santa Cruz, CA, USA) and a control siRNA (Cat #AM4636) was purchased from Applied Biosystems™ (Applied Biosystems™, Foster City, CA).

Cells were analyzed for protein expression by preparing Western blots of cell lysates 72 h post-transfection.

### LC/MS/MS Methods

All samples were analyzed on LC/MS/MS using an AB Sciex API 4000™ instrument, coupled to a Shimadzu LC-20AD LC Pump system. Analytical samples were separated using a Waters Atlantis T3 dC18 reverse phase HPLC column (20 mm×2.1 mm) (Atlantis® columns) at a flow rate of 0.5 mL/min. The mobile phase consisted of 0.1% formic acid in water (solvent A) and 0.1% formic acid in 100% acetonitrile (solvent B). Elution conditions are detailed in Table 1 below.

TABLE 1
LC/MS/MS GRADIENT CONDITIONS

| Time (min) | Flow (μL/min) | % A | % B |
|---|---|---|---|
| 0 | 500 | 98 | 2 |
| 0.30 | 500 | 98 | 2 |
| 1.40 | 500 | 2 | 98 |
| 2.00 | 500 | 2 | 98 |
| 2.01 | 500 | 98 | 2 |
| 2.50 | 500 | 98 | 2 |

### Statistical Analyses

Analyses were performed using GraphPad Prism version 9.0 software (GraphPad Software Inc. San Diego, CA). A P value of 0.05 was considered statistically significant. Results were compared using unpaired Student's t tests or one-way ANOVA with correction for multiple comparisons as indicated. The number of biological and technical replicates is indicated for each figure and presented as dot blots to show data distribution.

### Results

### Example 1: Design, Synthesis and Characterization of a Cell-Impermeable Staurosporine Analog

Hypothesizing that inhibition of Akt and other upstream or downstream activating kinases that have been translocated to the outer leaflet of the plasma membrane would modulate viral entry, the well-studied broad spectrum kinase inhibitor staurosporine was selected as a scaffold to develop an inhibitor that would selectively target extracellular kinases(9). As there was no extant structure of an Akt:staurosporine complex, a model was generated by overlaying the coordinates of Akt1 (one of three Akt isoforms) bound to a small molecule inhibitor (Protein Data Bank (PDB) entry 3MVH) with those of the related ribosomal protein S6 kinase beta-1, p70S6K1, bound to staurosporine (about 46% sequence identity; PDB entry 3A60). The structural alignment revealed close agreement between the protein coordinates (for 211 aligned Cu pairs, RMSD=1.0 Å). The secondary amine moiety of staurosporine appeared to be solvent accessible and unhindered in the resulting model and thus represented a candidate for synthetic elaboration to introduce a solvent exposed polar group, which would impair cell entry while maintaining Akt binding properties.

Based on this modeling, a synthetic scheme was devised to install a moiety bearing a sulfonate functionality at the 2° amine of staurosporine, yielding a cell-impermeable analogue of staurosporine (CIMSS) (FIG. 1A). We hypothesized that CIMSS would inhibit HSV entry without inducing apoptosis or interfering with intracellular Akt signaling pathways triggered by other stimuli such as activation of insulin receptors (Shomin et al., 2009, Bioorg Med Chem 17, 6196-6202). In an in vitro kinase inhibitor assay performed against a panel of 393 kinases with 10 μM ATP, CIMSS (10 μM) retained the pleiotropic inhibitory property of staurosporine, including inhibition of the three isoforms of Akt (Akt1, Akt2, Akt3) and PDPK1, the enzyme responsible for cytoplasmic phosphorylation of Akt isoforms, by >75% (see Table 2). Similar levels of inhibition were observed for a range of Src family kinases, Aurora kinases, JAK kinases and receptor tyrosine kinases. The 50% inhibitory concentration (IC₅₀) for CIMSS and staurosporine were comparable for PDPK1, for example, but CIMSS was less effective than staurosporine for other kinases, including insulin receptor, Akt1 and PKA (˜40, ˜230, and ˜46-fold less effective) (Table 3). The altered inhibitory activity of CIMSS is consistent with a previous report (Shomin et al., 2009), in which acylation of the 4′-methylamine negatively impacted IC₅₀s, with increases of 1-2 orders of magnitude for diverse kinases, including PIM (>200-fold), CHK1 (84-fold), CDK2 (50-fold) and PKA (14-fold).

TABLE 2
CIMSS (10 μM) WAS TESTED FOR ACTIVITY AGAINST
A PANEL OF 393 KINASES AND COMPARED TO
STAUROSPORINE OR INDICATED CONTROLS

| | % Enzyme Activity | | | |
|---|---|---|---|---|
| | (relative to | | IC₅₀ (M) | |
| | DMSO controls) | | of | |
| | CIMSS (10 μM) | | Control | Control |
| Kinase: | Data 1 | Data 2 | Compound | compound |
| ABL1 | 5.18 | 4.92 | 4.00E−08 | Staurosporine |
| ABL2/ARG | 4.07 | 3.90 | 1.19E−08 | Staurosporine |
| ACK1 | 7.33 | 5.60 | 5.68E−08 | Staurosporine |
| AKT1 | 23.73 | 23.61 | 7.80E−09 | Staurosporine |
| AKT2 | 29.76 | 19.09 | 2.61E−08 | Staurosporine |
| AKT3 | 11.41 | 9.86 | 1.77E−09 | Staurosporine |
| ALK | 4.12 | 3.76 | 3.17E−09 | Staurosporine |
| ALK1/ACVRL1 | 70.00 | 69.61 | 1.31E−08 | LDN193189 |
| ALK2/ACVR1 | 72.71 | 71.88 | 1.78E−08 | LDN193189 |
| ALK3/BMPR1A | 79.51 | 79.43 | 3.21E−08 | LDN193189 |
| ALK4/ACVR1B | 58.26 | 57.94 | 1.02E−07 | LDN193189 |
| ALK5/TGFBR1 | 86.58 | 85.77 | 2.13E−07 | LDN193189 |
| ALK6/BMPR1B | 88.27 | 87.18 | 1.22E−08 | LDN193189 |
| ARAF | 154.16 | 152.51 | 1.39E−08 | GW5074 |
| ARK5/NUAK1 | 1.83 | 1.45 | 2.96E−09 | Staurosporine |
| ASK1/MAP3K5 | 6.25 | 6.05 | 1.11E−08 | Staurosporine |
| Aurora A | 1.92 | 1.08 | 1.73E−09 | Staurosporine |
| Aurora B | 2.85 | 2.83 | 5.55E−09 | Staurosporine |
| AURORA C | 2.59 | 2.18 | 2.98E−09 | Staurosporine |
| AXL | 3.61 | 3.52 | 8.93E−09 | Staurosporine |
| BLK | 0.34 | 0.29 | 2.07E−09 | Staurosporine |
| BMPR2 | 50.39 | 49.89 | 2.00E−07 | Staurosporine |
| BMX/ETK | 2.19 | 1.32 | 6.88E−09 | Staurosporine |
| BRAF | 131.63 | 131.61 | 7.62E−09 | GW5074 |
| BRK | 25.50 | 24.16 | 1.74E−07 | Staurosporine |
| BRSK1 | 1.55 | 1.28 | 4.15E−10 | Staurosporine |
| BRSK2 | 1.66 | 1.53 | 2.53E−09 | Staurosporine |
| BTK | 6.64 | 5.98 | 3.00E−08 | Staurosporine |
| c-Kit | 0.28 | −0.14 | 7.38E−10 | Staurosporine |
| c-MER | 2.17 | 2.15 | 1.68E−08 | Staurosporine |
| c-MET | 12.40 | 9.84 | 1.77E−07 | Staurosporine |
| c-Src | 2.95 | 2.85 | 3.33E−09 | Staurosporine |
| CAMK1a | 28.74 | 28.61 | 5.53E−09 | Staurosporine |
| CAMK1b | 59.52 | 57.60 | 4.15E−09 | Staurosporine |
| CAMK1d | 20.31 | 19.69 | 3.17E−10 | Staurosporine |
| CAMK1g | 8.97 | 8.82 | 3.21E−09 | Staurosporine |
| CAMK2a | 1.36 | 1.34 | 6.56E−11 | Staurosporine |
| CAMK2b | 3.03 | 2.90 | 5.53E−11 | Staurosporine |
| CAMK2d | 1.33 | 0.88 | 5.24E−12 | Staurosporine |
| CAMK2g | 1.25 | 0.85 | 3.45E−10 | Staurosporine |
| CAMK4 | 66.61 | 62.68 | 1.32E−07 | Staurosporine |
| CAMKK1 | 1.00 | 1.00 | 5.39E−08 | Staurosporine |
| CAMKK2 | 1.86 | 1.81 | 7.53E−08 | Staurosporine |
| CDC7/DBF4 | 3.98 | 3.26 | 1.47E−08 | Staurosporine |
| CDK1/cyclin A | 21.33 | 18.54 | 3.80E−09 | Staurosporine |
| CDK1/cyclin B | 13.58 | 13.39 | 2.32E−09 | Staurosporine |
| CDK1/cyclin E | 17.98 | 17.80 | 2.02E−09 | Staurosporine |
| CDK14/cyclin Y | 74.48 | 72.89 | 1.33E−07 | Staurosporine |
| (PFTK1) | | | | |
| CDK16/cyclin Y | 76.57 | 76.42 | 3.18E−08 | Staurosporine |
| (PCTAIRE) | | | | |
| CDK17/cyclin Y | 54.17 | 52.31 | 1.03E−08 | Staurosporine |
| (PCTK2) | | | | |
| CDK18/cyclin Y | 41.31 | 36.57 | 1.24E−08 | Staurosporine |
| (PCTK3) | | | | |
| CDK19/cyclin C | 37.92 | 37.25 | 1.83E−07 | Staurosporine |
| CDK2/cyclin A | 5.14 | 4.94 | 1.30E−09 | Staurosporine |
| CDK2/cyclin A1 | 7.95 | 7.85 | 4.15E−09 | Staurosporine |
| CDK2/CYCLIN E | 7.89 | 5.60 | 2.27E−09 | Staurosporine |
| CDK2/cyclin E2 | 11.76 | 11.44 | 3.04E−09 | Staurosporine |
| CDK2/cyclin O | 4.97 | 4.80 | 2.40E−09 | Staurosporine |
| CDK3/cyclin E | 13.87 | 12.85 | 2.64E−09 | Staurosporine |
| CDK3/cyclin E2 | 22.31 | 20.66 | 4.31E−09 | Staurosporine |
| CDK4/cyclin D1 | 68.81 | 68.11 | 6.48E−09 | Staurosporine |
| CDK4/cyclin D3 | 85.64 | 84.60 | 1.71E−08 | Staurosporine |
| CDK5/P25 | 7.17 | 6.97 | 1.99E−09 | Staurosporine |
| CDK5/p35 | 8.78 | 8.70 | 1.95E−09 | Staurosporine |
| CDK6/cyclin D1 | 62.97 | 61.17 | 3.56E−09 | Staurosporine |
| CDK6/cyclin D3 | 56.76 | 54.89 | 9.33E−09 | Staurosporine |
| CDK7/cyclin H | 65.58 | 65.37 | 2.77E−08 | Staurosporine |
| CDK8/cyclin C | 10.10 | 10.03 | 5.22E−10 | Staurosporine |
| CDK9/cyclin K | 11.43 | 11.18 | 2.40E−08 | Staurosporine |
| CDK9/cyclin T1 | 13.12 | 13.12 | 5.90E−09 | Staurosporine |
| CDK9/cyclin T2 | 8.55 | 8.32 | 2.15E−09 | Staurosporine |
| CHK1 | 4.66 | 4.46 | 1.81E−10 | Staurosporine |
| CHK2 | 25.42 | 25.13 | 5.48E−09 | Staurosporine |
| CK1a1 | 98.34 | 97.55 | 4.02E−06 | Staurosporine |
| CK1a1L | 82.84 | 78.55 | 7.11E−07 | Staurosporine |
| CK1d | 106.16 | 100.57 | 1.49E−07 | D4476 |
| CK1epsilon | 38.82 | 38.79 | 2.61E−07 | D4476 |
| CK1g1 | 95.86 | 92.37 | 8.27E−06 | Staurosporine |
| CK1g2 | 98.52 | 93.32 | 1.47E−06 | Staurosporine |
| CK1g3 | 97.22 | 95.03 | 4.61E−06 | Staurosporine |
| CK2a | 71.37 | 70.67 | 2.67E−07 | GW5074 |
| CK2a2 | 44.65 | 43.00 | 1.41E−06 | Staurosporine |
| CLK1 | 4.05 | 3.79 | 1.14E−08 | Staurosporine |
| CLK2 | 4.13 | 3.10 | 3.46E−09 | Staurosporine |
| CLK3 | 35.40 | 34.93 | 1.17E−06 | Staurosporine |
| CLK4 | 10.44 | 10.10 | 5.24E−08 | Staurosporine |
| COT1/MAP3K8 | 109.68 | 103.54 | 7.47E−06 | Ro-31-8220 |
| CSK | 2.95 | 2.41 | 2.45E−08 | Staurosporine |
| CTK/MATK | 24.61 | 24.07 | 1.49E−07 | Staurosporine |
| DAPK1 | 13.62 | 13.55 | 2.48E−08 | Staurosporine |
| DAPK2 | 11.32 | 10.10 | 5.60E−09 | Staurosporine |
| DCAMKL1 | 83.72 | 80.77 | 7.10E−08 | Staurosporine |
| DCAMKL2 | 87.69 | 86.26 | 1.79E−08 | Staurosporine |
| DDR1 | 0.98 | 0.67 | 7.07E−09 | Staurosporine |
| DDR2 | 1.67 | 1.45 | 7.62E−10 | Staurosporine |
| DMPK | 76.55 | 73.32 | 1.91E−08 | Staurosporine |
| DMPK2 | 10.86 | 9.78 | 2.92E−10 | Staurosporine |
| DRAK1/STK17A | 18.20 | 17.55 | 2.89E−08 | Staurosporine |
| DYRK1/DYRK1A | 2.33 | 1.92 | 4.31E−09 | Staurosporine |
| DYRK1B | 0.41 | 0.35 | 8.42E−10 | Staurosporine |
| DYRK2 | 9.12 | 8.90 | 1.63E−07 | Staurosporine |
| DYRK3 | 28.20 | 27.70 | 4.48E−08 | Staurosporine |
| DYRK4 | 92.01 | 91.31 | 4.12E−06 | GW5074 |
| EGFR | 17.39 | 16.47 | 6.38E−08 | Staurosporine |
| EPHA1 | 31.47 | 30.84 | 1.59E−07 | Staurosporine |
| EPHA2 | 33.51 | 31.99 | 8.05E−08 | Staurosporine |
| EPHA3 | 37.88 | 37.82 | 9.15E−08 | Staurosporine |
| EPHA4 | 30.47 | 28.44 | 1.15E−08 | Staurosporine |
| EPHA5 | 52.72 | 52.25 | 1.26E−08 | Staurosporine |
| EPHA6 | 34.56 | 33.33 | 3.82E−08 | Staurosporine |
| EPHA7 | 6.91 | 6.08 | 8.59E−08 | Staurosporine |
| EPHA8 | 70.10 | 69.23 | 8.16E−08 | Staurosporine |
| EPHB1 | 1.91 | 1.62 | 3.21E−08 | Staurosporine |
| EPHB2 | 28.50 | 27.11 | 7.34E−08 | Staurosporine |
| EPHB3 | 28.89 | 28.88 | 5.97E−07 | Staurosporine |
| EPHB4 | 19.38 | 18.92 | 2.22E−07 | Staurosporine |
| ERBB2/HER2 | 35.89 | 34.05 | 1.15E−07 | Staurosporine |
| ERBB4/HER4 | 25.01 | 24.80 | 2.25E−07 | Staurosporine |
| ERK1 | 91.79 | 90.38 | 2.42E−09 | SCH772984 |
| ERK2/MAPK1 | 75.35 | 74.09 | 4.42E−10 | SCH772984 |
| ERK5/MAPK7 | 84.47 | 81.71 | 6.77E−06 | Staurosporine |
| ERK7/MAPK15 | 27.94 | 27.60 | 1.80E−08 | Staurosporine |
| ERN1/IRE1 | 8.49 | 8.17 | 8.11E−08 | Staurosporine |
| ERN2/IRE2 | 6.47 | 5.93 | 3.98E−08 | Staurosporine |
| FAK/PTK2 | 15.52 | 13.59 | 2.29E−08 | Staurosporine |
| FER | 0.37 | 0.26 | 9.21E−10 | Staurosporine |
| FES/FPS | 2.77 | 2.56 | 2.16E−09 | Staurosporine |
| FGFR1 | 6.31 | 6.17 | 7.30E−09 | Staurosporine |
| FGFR2 | 2.58 | 2.54 | 4.02E−09 | Staurosporine |
| FGFR3 | 4.66 | 4.46 | 1.36E−08 | Staurosporine |
| FGFR4 | 4.31 | 4.30 | 5.54E−08 | Staurosporine |
| FGR | 1.68 | 1.11 | 8.33E−10 | Staurosporine |
| FLT1/VEGFR1 | 2.92 | 2.85 | 3.66E−09 | Staurosporine |
| FLT3 | 4.55 | 4.52 | 1.27E−09 | Staurosporine |
| FLT4/VEGFR3 | 1.97 | 1.86 | 3.31E−09 | Staurosporine |
| FMS | 0.92 | 0.52 | 1.50E−09 | Staurosporine |
| FRK/PTK5 | 4.49 | 4.46 | 1.15E−08 | Staurosporine |
| FYN | 2.59 | 2.44 | 9.16E−10 | Staurosporine |
| GCK/MAP4K2 | 4.74 | 4.56 | 3.41E−10 | Staurosporine |
| GLK/MAP4K3 | 0.96 | 0.93 | 1.85E−10 | Staurosporine |
| GRK1 | 4.70 | 4.55 | 4.67E−08 | Staurosporine |
| GRK2 | 29.46 | 28.58 | 1.08E−06 | Staurosporine |
| GRK3 | 46.88 | 46.87 | 2.32E−06 | Staurosporine |
| GRK4 | 19.80 | 19.80 | 1.40E−07 | Staurosporine |
| GRK5 | 7.35 | 7.04 | 5.30E−08 | Staurosporine |
| GRK6 | 8.36 | 8.11 | 6.70E−08 | Staurosporine |
| GRK7 | 7.19 | 6.73 | 9.93E−09 | Staurosporine |
| GSK3a | 2.72 | 2.26 | 3.94E−09 | Staurosporine |
| GSK3b | 2.85 | 2.83 | 7.03E−09 | Staurosporine |
| Haspin | 49.47 | 49.36 | 1.61E−08 | Staurosporine |
| HCK | 7.23 | 6.46 | 3.68E−09 | Staurosporine |
| HGK/MAP4K4 | 0.44 | −0.12 | 4.86E−10 | Staurosporine |
| HIPK1 | 36.35 | 33.68 | 6.33E−07 | Ro-31-8220 |
| HIPK2 | 17.52 | 17.11 | 8.62E−07 | Staurosporine |
| HIPK3 | 14.49 | 13.73 | 8.01E−07 | Staurosporine |
| HIPK4 | 10.70 | 10.40 | 3.79E−07 | Staurosporine |
| HPK1/MAP4K1 | 2.66 | 2.01 | 4.22E−08 | Ro-31-8220 |
| IGF1R | 6.72 | 6.42 | 2.49E−08 | Staurosporine |
| IKKa/CHUK | 31.57 | 30.89 | 1.06E−07 | Staurosporine |
| IKKb/IKBKB | 33.35 | 31.51 | 2.57E−07 | Staurosporine |
| IKKe/IKBKE | 1.47 | 1.28 | 1.80E−10 | Staurosporine |
| IR | 15.79 | 15.41 | 2.41E−08 | Staurosporine |
| IRAK1 | 3.57 | 3.16 | 5.40E−08 | Staurosporine |
| IRAK4 | 6.48 | 6.41 | 3.45E−09 | Staurosporine |
| IRR/INSRR | 4.68 | 3.78 | 1.36E−08 | Staurosporine |
| ITK | 4.12 | 3.38 | 1.05E−08 | Staurosporine |
| JAK1 | −0.15 | −0.64 | 6.48E−10 | Staurosporine |
| JAK2 | 1.13 | 0.89 | 2.16E−10 | Staurosporine |
| JAK3 | 0.97 | 0.84 | 1.46E−10 | Staurosporine |
| JNK1 | 24.84 | 24.80 | 3.97E−07 | Staurosporine |
| JNK2 | 42.32 | 41.78 | 1.23E−06 | Staurosporine |
| JNK3 | 23.55 | 23.39 | 3.66E−08 | JNKi VIII |
| KDR/VEGFR2 | 7.22 | 6.16 | 2.19E−08 | Staurosporine |
| KHS/MAP4K5 | 5.19 | 5.14 | 2.44E−10 | Staurosporine |
| KSR1 | 90.75 | 86.36 | 6.62E−06 | Staurosporine |
| KSR2 | 107.87 | 107.01 | 6.83E−06 | Staurosporine |
| LATS1 | 9.89 | 9.79 | 1.82E−08 | Staurosporine |
| LATS2 | 6.54 | 6.32 | 7.87E−09 | Staurosporine |
| LCK | 3.41 | 2.99 | 2.35E−09 | Staurosporine |
| LCK2/ICK | 4.57 | 4.44 | 1.32E−08 | Staurosporine |
| LIMK1 | 0.88 | 0.77 | 1.27E−09 | Staurosporine |
| LIMK2 | 67.42 | 66.53 | 7.48E−08 | Staurosporine |
| LKB1 | 37.96 | 37.39 | 5.16E−08 | Staurosporine |
| LOK/STK10 | 3.02 | 2.86 | 4.81E−08 | Ro-31-8220 |
| LRRK2 | 1.69 | 1.00 | 7.30E−09 | Staurosporine |
| LYN | 2.50 | 1.83 | 6.59E−10 | Staurosporine |
| LYN B | 2.95 | 2.48 | 3.59E−09 | Staurosporine |
| MAK | 13.19 | 12.70 | 1.24E−08 | Staurosporine |
| MAPKAPK2 | 69.90 | 67.54 | 1.41E−07 | Staurosporine |
| MAPKAPK3 | 87.53 | 85.18 | 5.82E−06 | Staurosporine |
| MAPKAPK5/PRAK | 85.17 | 84.10 | 3.01E−07 | Staurosporine |
| MARK1 | 1.18 | 1.16 | 1.22E−10 | Staurosporine |
| MARK2/PAR-1Ba | 2.38 | 2.21 | 6.75E−11 | Staurosporine |
| MARK3 | 1.80 | 1.76 | 2.48E−10 | Staurosporine |
| MARK4 | 0.77 | 0.72 | 5.41E−10 | Staurosporine |
| MAST3 | 106.09 | 104.37 | 6.25E−07 | Staurosporine |
| MASTL | 33.79 | 33.66 | 4.46E−08 | Staurosporine |
| MEK1 | 76.66 | 75.35 | 5.41E−08 | Staurosporine |
| MEK2 | 67.99 | 66.36 | 5.15E−08 | Staurosporine |
| MEK3 | 49.31 | 47.29 | 1.10E−08 | Staurosporine |
| MEK5 | 40.44 | 40.06 | 3.29E−08 | Staurosporine |
| MEKK1 | 66.97 | 65.72 | 7.69E−07 | Staurosporine |
| MEKK2 | 8.32 | 8.26 | 4.15E−08 | Staurosporine |
| MEKK3 | 6.32 | 5.56 | 2.49E−08 | Staurosporine |
| MEKK6 | 110.20 | 107.81 | 5.29E−07 | Staurosporine |
| MELK | 1.43 | 1.31 | 1.17E−09 | Staurosporine |
| MINK/MINK1 | −1.70 | −1.95 | 7.78E−10 | Staurosporine |
| MKK4 | 99.00 | 97.17 | 1.86E−06 | Staurosporine |
| MKK6 | 59.69 | 57.24 | 1.02E−08 | Staurosporine |
| MKK7 | 96.21 | 94.42 | 2.75E−06 | Staurosporine |
| MLCK/MYLK | 15.47 | 14.41 | 2.13E−08 | Staurosporine |
| MLCK2/MYLK2 | 27.32 | 24.42 | 3.22E−08 | Staurosporine |
| MLK1/MAP3K9 | 4.06 | 4.01 | 4.67E−10 | Staurosporine |
| MLK2/MAP3K10 | 1.86 | 1.47 | 3.12E−09 | Staurosporine |
| MLK3/MAP3K11 | −0.81 | −1.50 | 2.12E−09 | Staurosporine |
| MLK4 | 0.87 | 0.85 | 1.56E−06 | Staurosporine |
| MNK1 | 42.65 | 41.13 | 1.06E−07 | Staurosporine |
| MNK2 | 4.79 | 4.76 | 1.35E−08 | Staurosporine |
| MRCKa/CDC42BPA | 71.26 | 68.39 | 4.63E−09 | Staurosporine |
| MRCKb/CDC42BPB | 75.84 | 73.82 | 3.81E−09 | Staurosporine |
| MSK1/RPS6KA5 | 1.53 | 1.49 | 9.57E−10 | Staurosporine |
| MSK2/RPS6KA4 | 2.11 | 1.99 | 2.93E−09 | Staurosporine |
| MSSK1/STK23 | 67.66 | 67.63 | 1.31E−06 | Staurosporine |
| MST1/STK4 | 4.33 | 3.39 | 1.56E−09 | Staurosporine |
| MST2/STK3 | 3.52 | 3.40 | 1.97E−09 | Staurosporine |
| MST3/STK24 | 16.65 | 15.83 | 1.25E−08 | Staurosporine |
| MST4 | 1.35 | 1.22 | 4.52E−09 | Staurosporine |
| MUSK | 3.33 | 2.02 | 4.56E−09 | Staurosporine |
| MYLK3 | 67.58 | 64.65 | 8.66E−08 | Staurosporine |
| MYLK4 | 6.48 | 5.38 | 3.97E−08 | Staurosporine |
| MYO3A | 6.46 | 5.96 | 2.63E−08 | Staurosporine |
| MYO3b | 5.12 | 4.81 | 9.47E−09 | Staurosporine |
| NEK1 | 5.88 | 5.78 | 1.90E−08 | Staurosporine |
| NEK11 | 52.04 | 49.43 | 1.23E−06 | Staurosporine |
| NEK2 | 18.82 | 18.42 | 2.31E−07 | Staurosporine |
| NEK3 | 87.31 | 85.95 | 5.46E−08 | JNK-IN-7 |
| NEK4 | 6.24 | 5.73 | 1.48E−07 | Staurosporine |
| NEK5 | 6.56 | 6.22 | 3.04E−08 | Staurosporine |
| NEK6 | 89.04 | 87.72 | 9.32E−06 | PKR Inhibitor |
| NEK7 | 88.55 | 82.08 | 8.35E−06 | PKR Inhibitor |
| NEK9 | 2.81 | 2.70 | 1.28E−07 | Staurosporine |
| NIM1 | 46.64 | 46.56 | 1.49E−07 | Staurosporine |
| NLK | 72.95 | 68.44 | 1.32E−07 | Staurosporine |
| OSR1/OXSR1 | 54.24 | 53.02 | 9.91E−08 | Staurosporine |
| P38a/MAPK14 | 101.68 | 100.67 | 2.18E−08 | SB202190 |
| P38b/MAPK11 | 84.51 | 83.74 | 3.20E−08 | SB202190 |
| P38d/MAPK13 | 74.74 | 69.92 | 2.04E−07 | Staurosporine |
| P38g | 30.29 | 29.80 | 1.58E−07 | Staurosporine |
| p70S6K/RPS6KB1 | 5.46 | 4.80 | 3.34E−10 | Staurosporine |
| p70S6Kb/RPS6KB2 | 3.38 | 3.26 | 1.56E−09 | Staurosporine |
| PAK1 | 8.08 | 7.21 | 3.31E−10 | Staurosporine |
| PAK3 | 6.48 | 6.27 | 1.61E−10 | Staurosporine |
| PAK4 | 7.78 | 7.24 | 2.86E−09 | Staurosporine |
| PAK5 | 7.72 | 7.41 | 4.99E−09 | Staurosporine |
| PAK6 | 9.79 | 8.98 | 5.52E−09 | Staurosporine |
| PASK | 41.61 | 40.24 | 1.54E−08 | Staurosporine |
| PBK/TOPK | 69.77 | 67.76 | 1.82E−07 | Staurosporine |
| PDGFRa | 6.05 | 5.54 | 1.62E−09 | Staurosporine |
| PDGFRb | 1.90 | 1.79 | 6.91E−09 | Staurosporine |
| PDK1/PDPK1 | 4.24 | 4.20 | 1.16E−09 | Staurosporine |
| PHKg1 | 3.74 | 3.44 | 9.56E−10 | Staurosporine |
| PHKg2 | 18.37 | 17.90 | 1.62E−09 | Staurosporine |
| PIM1 | 0.88 | 0.79 | 2.15E−09 | Staurosporine |
| PIM2 | 21.27 | 20.94 | 1.78E−08 | Staurosporine |
| PIM3 | 2.51 | 2.18 | 1.98E−10 | Staurosporine |
| PKA | 6.32 | 6.28 | 2.65E−09 | Staurosporine |
| PKAcb | 1.62 | 1.23 | 7.73E−10 | Staurosporine |
| PKAcg | 2.07 | 2.04 | 2.38E−09 | Staurosporine |
| PKCa | 0.86 | 0.84 | 3.85E−10 | Staurosporine |
| PKCb1 | 2.83 | 2.30 | 6.82E−09 | Staurosporine |
| PKCb2 | 1.71 | 1.66 | 4.28E−09 | Staurosporine |
| PKCd | 8.64 | 7.89 | 2.17E−10 | Staurosporine |
| PKCepsilon | 1.11 | 0.77 | 2.90E−10 | Staurosporine |
| PKCeta | 1.78 | 0.42 | 3.54E−10 | Staurosporine |
| PKCg | 1.77 | 1.53 | 1.57E−10 | Staurosporine |
| PKCIOTA | 31.54 | 30.93 | 2.26E−08 | Staurosporine |
| PKCmu/PRKD1 | 30.28 | 28.32 | 2.38E−09 | Staurosporine |
| PKCnu/PRKD3 | 11.43 | 11.12 | 2.32E−09 | Staurosporine |
| PKCtheta | 7.08 | 6.89 | 2.55E−09 | Staurosporine |
| PKCzeta | 55.85 | 55.80 | 6.22E−08 | Staurosporine |
| PKD2/PRKD2 | 11.93 | 11.75 | 1.51E−09 | Staurosporine |
| PKGla | 7.02 | 6.41 | 1.46E−09 | Staurosporine |
| PKG1b | 13.87 | 12.45 | 5.59E−09 | Staurosporine |
| PKG2/PRKG2 | 1.99 | 1.33 | 2.70E−09 | Staurosporine |
| PKN1/PRK1 | 2.22 | 2.15 | 1.39E−09 | Staurosporine |
| PKN2/PRK2 | 6.62 | 6.60 | 3.97E−09 | Staurosporine |
| PKN3/PRK3 | 20.17 | 14.75 | 8.93E−09 | Staurosporine |
| PLK1 | 17.14 | 17.05 | 1.56E−07 | Staurosporine |
| PLK2 | 27.38 | 26.59 | 2.22E−07 | Staurosporine |
| PLK3 | 98.18 | 93.76 | 2.62E−09 | BI2536 |
| PLK4/SAK | 6.73 | 6.38 | 1.44E−08 | Staurosporine |
| PRKX | 9.56 | 9.38 | 9.62E−10 | Staurosporine |
| PYK2 | 2.40 | 2.27 | 1.13E−08 | Staurosporine |
| RAF1 | 111.46 | 109.90 | 7.01E−09 | GW5074 |
| RET | 2.10 | 2.10 | 2.82E−09 | Staurosporine |
| RIPK2 | 41.72 | 40.54 | 1.16E−07 | Staurosporine |
| RIPK4 | 99.11 | 98.47 | 5.11E−07 | Staurosporine |
| RIPK5 | 22.66 | 22.32 | 2.88E−08 | Staurosporine |
| ROCK1 | 26.20 | 25.74 | 1.37E−09 | Staurosporine |
| ROCK2 | 12.69 | 12.53 | 8.88E−10 | Staurosporine |
| RON/MSTIR | 25.08 | 25.03 | 1.39E−07 | Staurosporine |
| ROS/ROS1 | 4.57 | 4.19 | 2.52E−10 | Staurosporine |
| RSK1 | 3.27 | 2.95 | 1.61E−10 | Staurosporine |
| RSK2 | 1.69 | 1.58 | 4.93E−10 | Staurosporine |
| RSK3 | 3.38 | 3.24 | 1.52E−10 | Staurosporine |
| RSK4 | 6.64 | 6.54 | 1.29E−10 | Staurosporine |
| SBK1 | 78.83 | 76.94 | 6.01E−08 | Staurosporine |
| SGK1 | 13.38 | 12.71 | 1.56E−08 | Staurosporine |
| SGK2 | 22.64 | 22.07 | 9.55E−09 | Staurosporine |
| SGK3/SGKL | 59.57 | 58.53 | 1.19E−07 | Staurosporine |
| SIK1 | 1.00 | 0.64 | 1.60E−09 | Staurosporine |
| SIK2 | 1.49 | 1.17 | 4.95E−10 | Staurosporine |
| SIK3 | 0.42 | 0.08 | 1.53E−09 | Staurosporine |
| SLK/STK2 | 6.42 | 6.42 | 2.31E−08 | Staurosporine |
| SNARK/NUAK2 | 2.16 | 1.92 | 2.73E−09 | Staurosporine |
| SNRK | 72.54 | 72.07 | 2.86E−08 | Staurosporine |
| SRMS | 90.96 | 90.39 | 7.05E−06 | Staurosporine |
| SRPK1 | 9.17 | 8.28 | 2.21E−08 | Staurosporine |
| SRPK2 | 8.02 | 7.62 | 1.40E−07 | Staurosporine |
| SSTK/TSSK6 | 95.67 | 94.64 | 2.17E−07 | Staurosporine |
| STK16 | 10.21 | 9.79 | 2.51E−07 | Staurosporine |
| STK21/CIT | 81.66 | 81.15 | 1.51E−07 | Staurosporine |
| STK22D/TSSK1 | 2.71 | 2.56 | 5.31E−11 | Staurosporine |
| STK25/YSK1 | 4.17 | 3.00 | 9.64E−09 | Staurosporine |
| STK32B/YANK2 | 0.96 | 0.91 | 2.90E−08 | Staurosporine |
| STK32C/YANK3 | 20.52 | 20.31 | 2.42E−07 | Staurosporine |
| STK33 | 9.17 | 8.22 | 3.41E−08 | Staurosporine |
| STK38/NDR1 | 9.08 | 8.29 | 5.74E−10 | Staurosporine |
| STK38L/NDR2 | 22.67 | 22.41 | 3.00E−09 | Staurosporine |
| STK39/STLK3 | 19.65 | 18.27 | 1.90E−08 | Staurosporine |
| SYK | 0.87 | 0.78 | 9.24E−10 | Staurosporine |
| TAK1 | 7.20 | 6.81 | 6.09E−08 | Staurosporine |
| TAOK1 | 6.83 | 6.33 | 2.05E−09 | Staurosporine |
| TAOK2/TAO1 | 36.34 | 35.06 | 6.77E−09 | Staurosporine |
| TAOK3/JIK | 12.84 | 12.63 | 7.23E−09 | Staurosporine |
| TBK1 | 2.01 | 1.90 | 1.00E−09 | Staurosporine |
| TEC | 19.95 | 19.75 | 6.93E−08 | Staurosporine |
| TESK1 | 82.95 | 82.57 | 3.60E−07 | Staurosporine |
| TESK2 | 91.15 | 89.59 | 5.55E−06 | Staurosporine |
| TGFBR2 | 110.71 | 108.40 | 1.09E−07 | LDN193189 |
| TIE2/TEK | 7.56 | 7.23 | 6.00E−08 | Staurosporine |
| TLK1 | 26.05 | 25.57 | 2.22E−08 | Staurosporine |
| TLK2 | 67.01 | 66.39 | 3.94E−09 | Staurosporine |
| TNIK | 1.22 | 1.13 | 6.79E−10 | Staurosporine |
| TNK1 | 3.88 | 2.88 | 5.28E−09 | Staurosporine |
| TRKA | −2.22 | −3.31 | 8.72E−09 | Staurosporine |
| TRKB | 1.79 | 1.73 | 3.73E−10 | Staurosporine |
| TRKC | 0.34 | 0.18 | 1.17E−10 | Staurosporine |
| TSSK2 | 21.20 | 21.04 | 1.15E−08 | Staurosporine |
| TSSK3/STK22C | 62.20 | 61.46 | 8.16E−09 | Staurosporine |
| TTBK1 | 67.65 | 66.32 | 6.89E−06 | SB202190 |
| TTBK2 | 95.09 | 94.91 | 4.31E−06 | SB202190 |
| TXK | 3.64 | 3.62 | 3.58E−08 | Staurosporine |
| TYK1/LTK | 7.18 | 6.71 | 2.38E−08 | Staurosporine |
| TYK2 | 6.35 | 5.86 | 2.50E−10 | Staurosporine |
| TYRO3/SKY | 23.63 | 23.35 | 6.56E−09 | Staurosporine |
| ULK1 | 13.58 | 13.46 | 9.04E−09 | Staurosporine |
| ULK2 | 13.64 | 13.54 | 3.44E−09 | Staurosporine |
| ULK3 | 14.16 | 11.94 | 3.38E−09 | Staurosporine |
| VRK1 | 81.57 | 79.87 | 7.34E−07 | Ro-31-8220 |
| VRK2 | 81.76 | 81.67 | 1.70E−05 | Ro-31-8220 |
| WEE1 | 44.25 | 43.28 | 3.59E−08 | Wee-1 Inhibitor |
| WNK1 | 109.55 | 107.75 | 4.83E−05 | Staurosporine |
| WNK2 | 71.92 | 69.29 | 4.74E−06 | Staurosporine |
| WNK3 | 66.36 | 64.44 | 1.49E−06 | Wee-1 Inhibitor |
| YES/YES1 | 1.40 | 1.32 | 2.01E−09 | Staurosporine |
| YSK4/MAP3K19 | 3.40 | 3.38 | 8.57E−09 | Staurosporine |
| ZAK/MLTK | 82.68 | 82.32 | 1.89E−06 | GW5074 |
| ZAP70 | 12.98 | 12.64 | 1.94E−08 | Staurosporine |
| ZIPK/DAPK3 | 6.48 | 5.05 | 4.17E−09 | Staurosporine |
| AMPK (A1/B2/G2) | −0.06 | −0.34 | 6.35E−08 | Ro-31-8220 |
| AMPK (A1/B2/G3) | 1.13 | 1.09 | 1.15E−07 | Ro-31-8220 |
| AMPK (A2/B1/G2) | 0.66 | 0.09 | 1.06E−07 | Ro-31-8220 |
| AMPK (A2/B1/G3) | 0.67 | 0.59 | 2.96E−07 | Ro-31-8220 |
| AMPK(A1/B1/G1) | 1.14 | 1.14 | 6.68E−08 | Ro-31-8220 |
| AMPK(A1/B1/G2) | 1.46 | 1.23 | 7.68E−08 | Ro-31-8220 |
| AMPK(A1/B1/G3) | 0.22 | 0.07 | 1.79E−07 | Ro-31-8220 |
| AMPK(A1/B2/G1) | 0.73 | 0.57 | 1.16E−07 | Ro-31-8220 |
| AMPK(A2/B1/G1) | 0.76 | 0.64 | 2.12E−07 | Ro-31-8220 |
| AMPK(A2/B2/G1) | 0.34 | 0.23 | 9.78E−08 | Ro-31-8220 |
| AMPK(A2/B2/G2) | 0.73 | 0.63 | 1.00E−07 | Ro-31-8220 |
| AMPK(A2/B2/G3) | 0.63 | 0.61 | 1.86E−07 | Ro-31-8220 |
| DNA-PK | 98.20 | 95.76 | 1.38E−08 | PI-103 |
| EEF2K | 99.51 | 97.05 | 7.40E−06 | NH125 |
| EIF2AK1 | 82.48 | 81.15 | 9.62E−08 | GSK-2606414 |
| EIF2AK2 | 9.89 | 9.34 | 5.80E−08 | Staurosporine |
| EIF2AK3 | 61.60 | 60.18 | 2.76E−09 | GSK-2606414 |
| EIF2AK4 | 9.50 | 9.39 | 7.51E−07 | Staurosporine |
| mTOR/FRAP1 | 111.59 | 107.76 | 5.01E−08 | PI-103 |
| PDK1/PDHK1 | 97.24 | 96.82 | 8.63E−06 | GW5074 |
| PDK2/PDHK2 | 95.46 | 93.95 | 5.05E−06 | GW5074 |
| PDK3/PDHK3 | 96.40 | 94.81 | 2.57E−06 | GW5074 |
| PDK4/PDHK4 | 93.19 | 92.98 | 1.89E−06 | GW5074 |
| TRPM7/CHAK1 | 91.59 | 91.48 | 2.34E−05 | NH125 |

TABLE 3
CONCENTRATION OF CIMSS AND STAUROSPORINE
THAT INHIBITED IN VITRO KINASE ACTIVITY BY 50% (IC₅₀)

| | Compound IC₅₀ (M) | |
|---|---|---|
| Kinase | CIMSS | Staurosporine |
| ABL1 | 1.43E−07 | 2.22E−08 |
| AKT1 | 4.61E−07 | 2.00E−09 |
| AKT2 | 1.00E−06 | 1.42E−08 |
| AKT3 | 1.57E−07 | 1.54E−09 |
| c-Src | 1.19E−08 | 1.61E−09 |
| EGFR | 1.61E−06 | 9.41E−08 |
| Insulin Receptor | 8.03E−07 | 1.86E−08 |
| PDPK1 | 2.78E−10 | 5.10E−10 |
| PKA | 4.23E−08 | 9.12E−10 |
| PKCd | 3.64E−09 | 1.27E−10 |

The low permeability of CIMSS compared to control compounds was demonstrated by measuring the apparent permeability coefficient (Papp) with confluent MDCK cells as well as in an artificial membrane permeability assay (PAMPA) (see Table 4 below). This behavior was further assessed in cell viability studies, which demonstrated that unmodified staurosporine significantly inhibited HaCat (human keratinocyte) cell growth at concentrations as low as 1 μM at 24 hours (h) and 0.1 μM at 72 h, whereas no significant inhibition was observed with CIMSS except when cells were cultured for 72 h in media containing 100 μM CIMSS (FIG. 1B). Similarly, little or no cytotoxicity was observed when fully confluent HaCat cells were incubated with 10 μM CIMSS or when about 50% confluent primary vaginal epithelial cells were cultured with 10 or 50 μM CIMSS for up to 120 h, whereas 10 μM staurosporine was cytotoxic within 24 h of culture (FIGS. 1C and D).

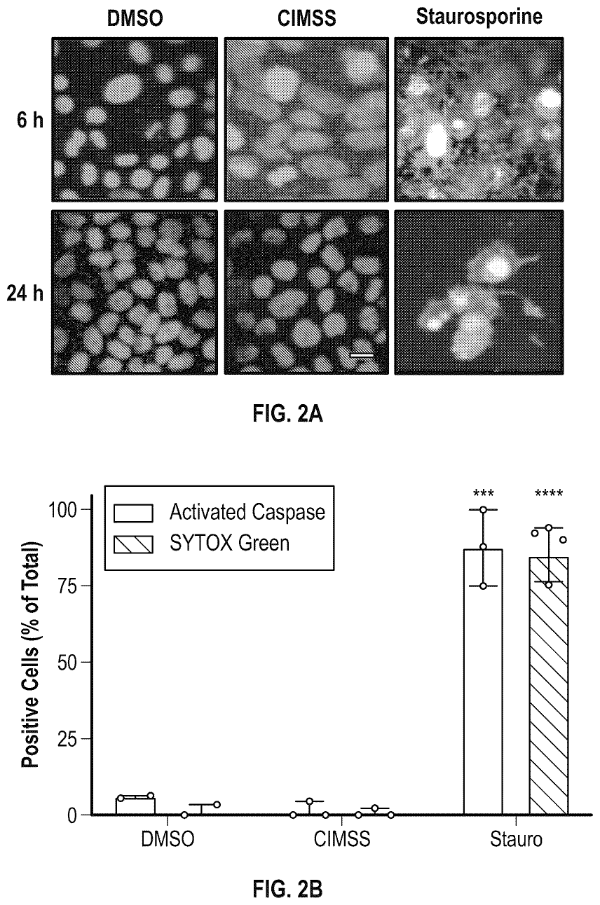

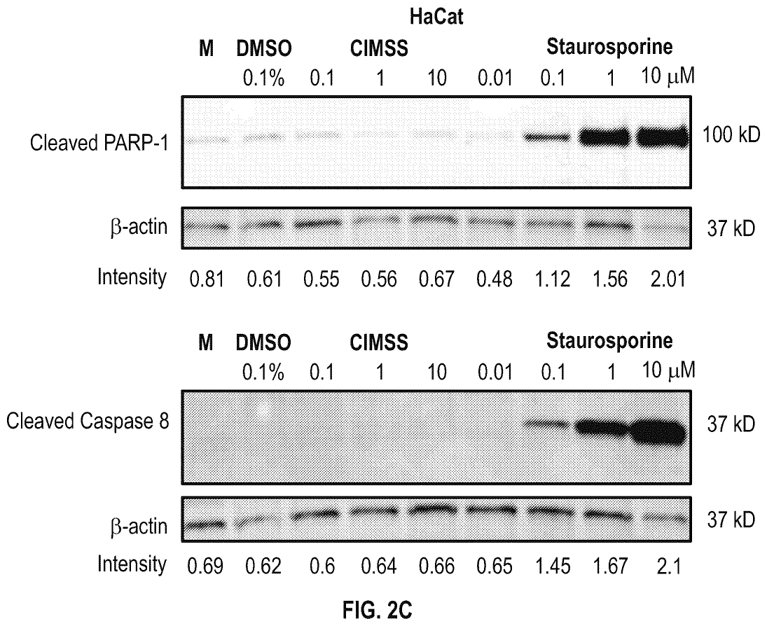

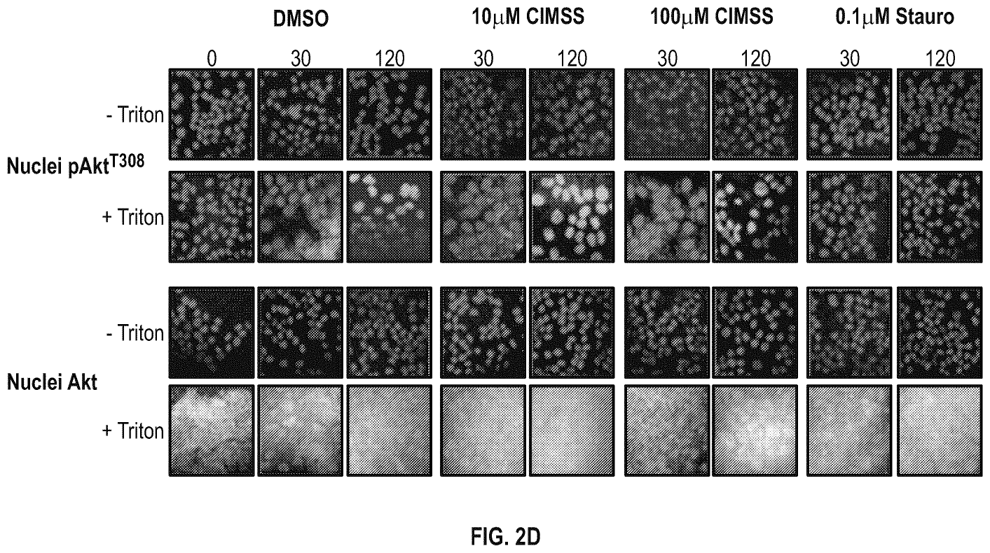

Consistent with its cell impermeability, CIMSS did not induce apoptosis, as measured by SYTOX Green and anti-caspase antibody staining (FIGS. 2A and B). Similarly, compared to cells treated with 0.1 μM DMSO, CIMSS at doses of 0.1, 1 or 10 μM did not induce PARP-1 or caspase 8 cleavage whereas cleavage of both was observed with staurosporine at concentrations as low as 0.1 μM (FIG. 2C). Furthermore, CIMSS did not block Akt phosphorylation in response to insulin. HaCat cells were treated with 10 μM insulin in the absence or presence of CIMSS (10 or 100 μM) or staurosporine (0.1 μM), fixed with or without Triton-X permeabilization and stained with conjugated antibodies to detect phosphorylated Akt (pAkt^{t308}) (red) or total Akt (green); nuclei were stained with DAPI (blue). We used higher doses of CIMSS than staurosporine based on the in vitro kinase activity (Table 2). Following insulin treatment, Akt was only visualized in permeabilized cells, and its intracellular phosphorylation was inhibited by staurosporine at doses as low as 0.1 μM, but not by 100 μM CIMSS (FIG. 2C). These findings indicate that engagement of the insulin receptor by insulin does not trigger translocation of Akt to the outer leaflet of the plasma membrane and that phosphorylation of cytoplasmic Akt is inhibited by staurosporine but is insensitive to CIMSS. These observations, together with direct permeability measurements and lack of apoptotic induction, are consistent with our proposition that CIMSS is membrane impermeable.

TABLE 4
PERMEABILITY STUDIES FOR CIMSS RELATIVE
TO CONTROL COMPOUNDS
MDCK Transport*

| Compound | Conc. [μM] | Papp | Recover % |
|---|---|---|---|
| Digoxin | 5 | 3.1 | 96 |
| Propranolol | 5 | 67.4 | 93 |
| CIMSS | 5 | 0.1 | 96 |
| Parallel artificial membrane permeability assay | | | |
| (PAMPA)* | | | |
| Propranolol | 100 | 8.991 | 85 |
| Atenolol | 100 | 0.001 | 96 |
| CIMSS | 100 | 0.005 | 83 |

*The apparent permeability coefficient (Papp) was calculated as the average two measurements; ×10⁻⁶ cm/s
**PAMPA was calculated as the average of four measurements; ×10⁻⁶ cm/s

### Example 2: CIMSS Inhibits HSV Infection and Blocks the Viral Induced Phosphorylation of Akt

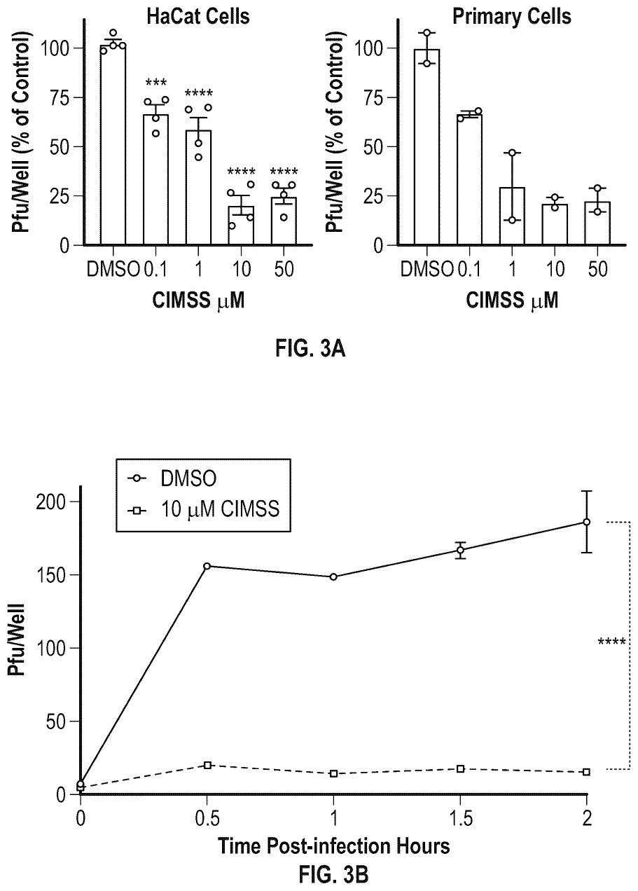

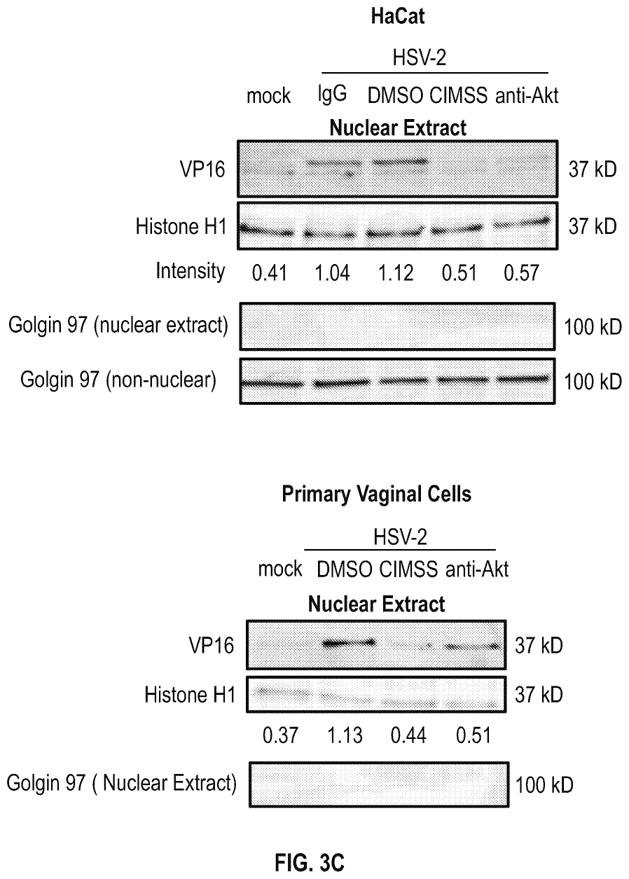

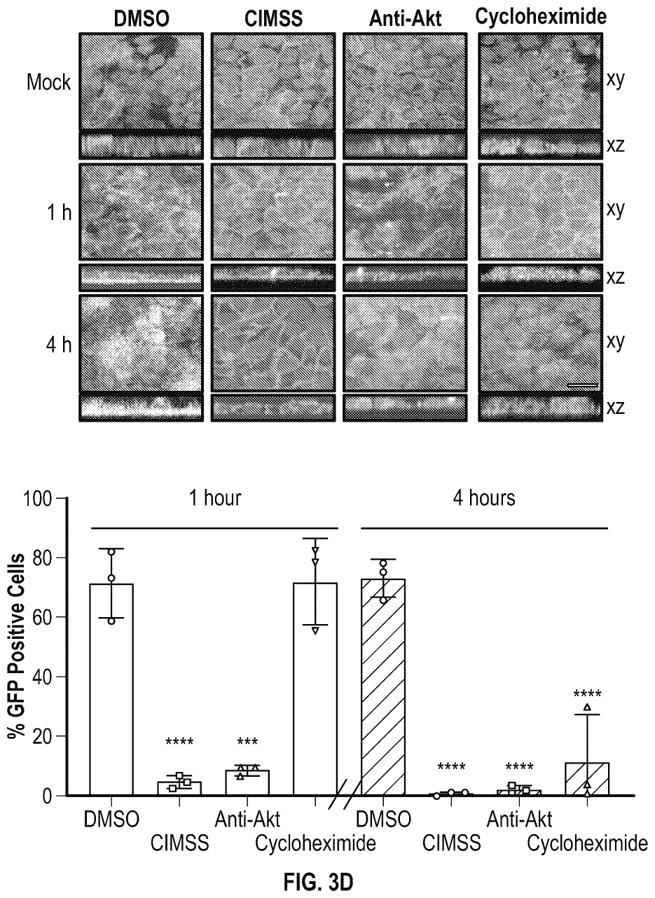

Given the association of Akt translocation and phosphorylation with HSV cell entry, we examined whether CIMSS would inhibit HSV infection by plaque assay and found that it inhibited HSV-2 infection of HaCat and primary vaginal cells in a dose dependent manner with >75% inhibition at a dose of 10 μM (FIG. 3A). Based on these results and the cell viability data, 10 μM CIMSS was used for subsequent infection studies. The inhibitory effects of 10 μM CIMSS or antibodies to Akt on HSV entry were assessed using complementary assays. First, the kinetics of viral entry were compared using a synchronized infection assay. HaCat cells were exposed to 150-200 pfu/well of HSV-2(G) at 4° C. for 4 h to allow virus to bind, washed, transferred to 37° C. (a temperature permissive for viral entry) in fresh media containing 10 μM CIMSS or DMSO. At the indicated times post-temperature shift (0.5, 1, 1.5 or 2 hours), the cells were treated with a low pH buffer to inactivate any extracellular virus, washed, overlaid with methylcellulose and viral plaques counted at 48 hours. The addition of CIMSS at each timepoint during the viral entry period resulted in a significant reduction in viral plaque formation (FIG. 3B, p<0.001). Second, the effects of CIMSS or anti-Akt antibodies on nuclear transport of the viral tegument protein, VP-16, a surrogate marker for viral entry, were assessed by preparing immunoblots of nuclear extracts 1 h post-infection (John, M et al., 2005, J Infect Dis 192, 1731-1740). The blots were also stained with an antibody to histone 1 (nuclear marker) and anti-golgin-97 (cytoplasmic marker). CIMSS and anti-Akt IgG (but not control IgG) inhibited the nuclear transport of VP-16 in HaCat and primary vaginal cells (FIG. 3C). The effects of CIMSS or anti-Akt antibodies on viral entry were also assessed by quantifying viral capsid transport to the nuclear pore by confocal imaging using a previously described assay (Cheshenko, N et al., 2005, J Biol Chem 280, 31116-31125). Cells were synchronously infected with HSV-1 K26GFP, which encodes green fluorescent protein fused to VP26 (13), and entry monitored by fixing and staining the cells 1 and 4 post temperature shift. To differentiate capsid entry from newly synthesized protein, cycloheximide, which blocks protein synthesis, was included as a control. At one hour post temperature shift, the percentage of GFP+ cells was significantly decreased by pretreatment with CIMSS or rabbit-anti-human Akt (p<0.001), but not by cycloheximide. In contrast, the percentage of GFP+ cells was significantly reduced by all three treatments 4 h post-temperature shift reflecting inhibition of entry (CMSS and anti-Akt) and inhibition of new protein synthesis (cycloheximide) (p<0.0001) (FIG. 3D).

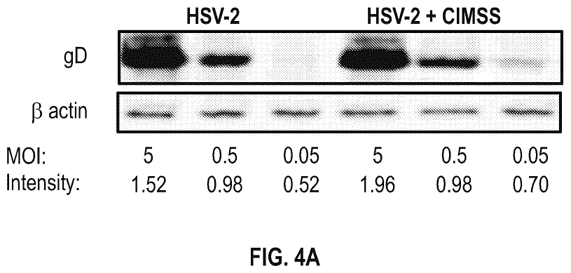

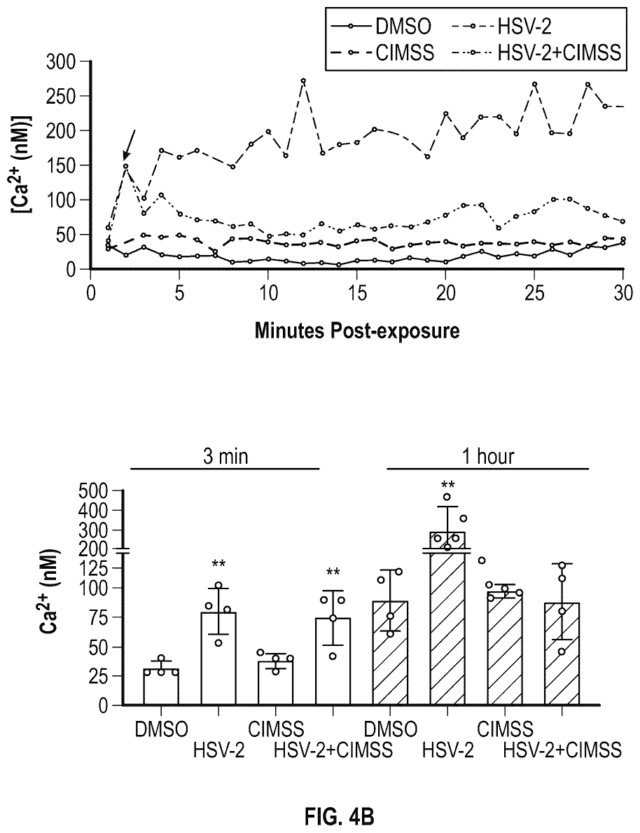

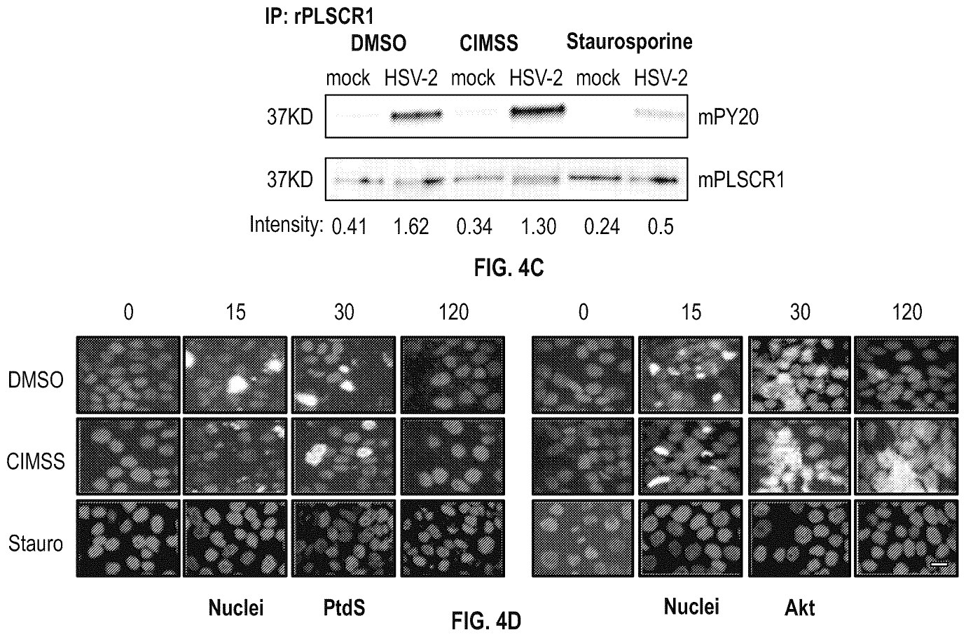

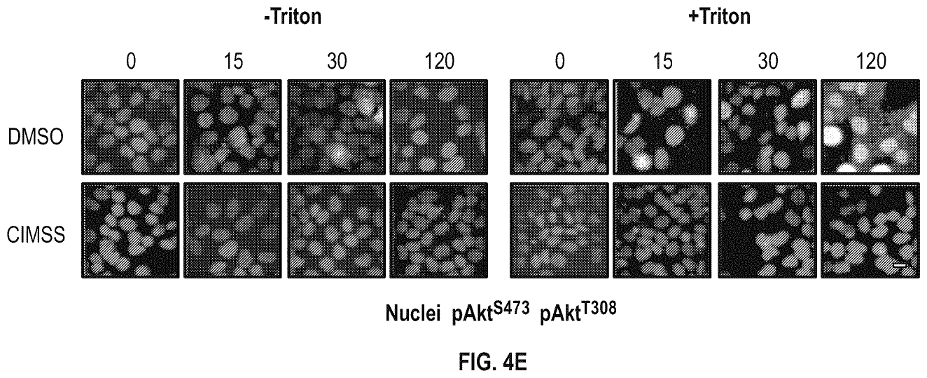

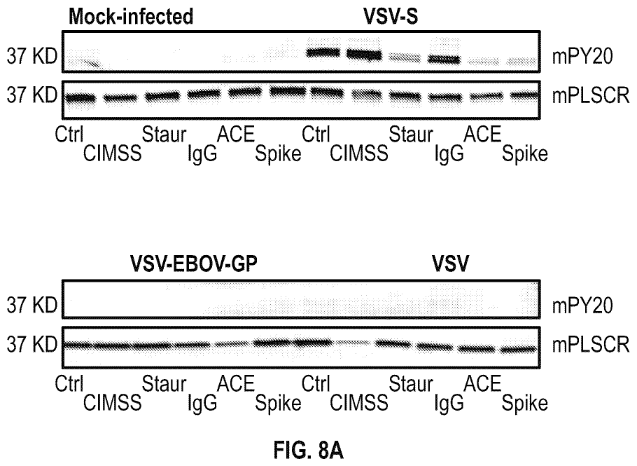

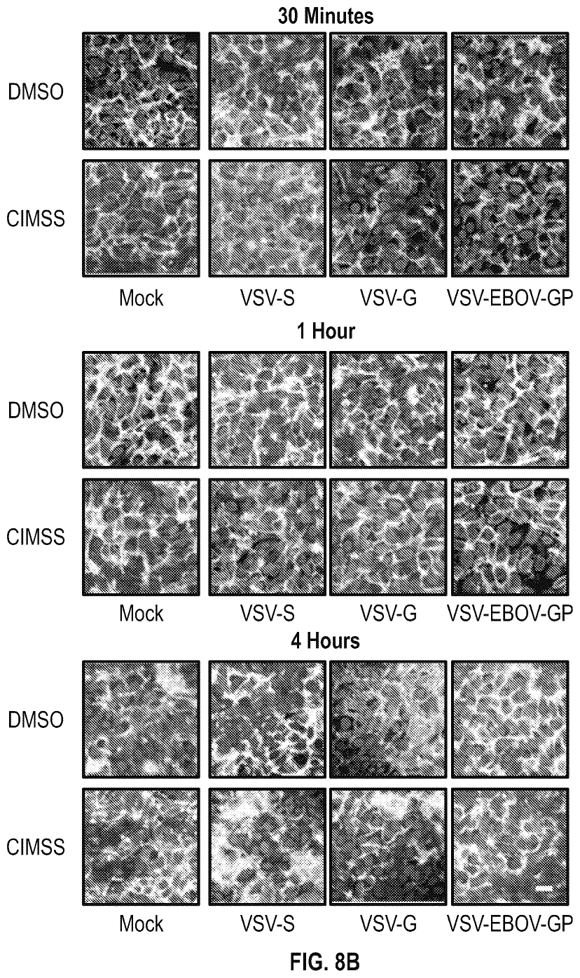

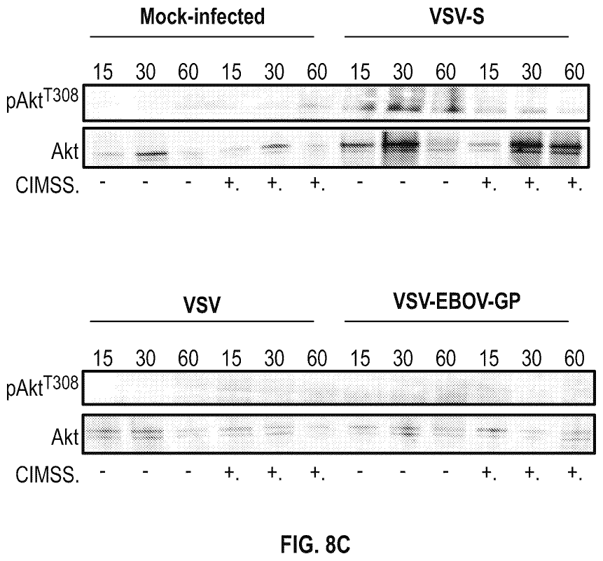

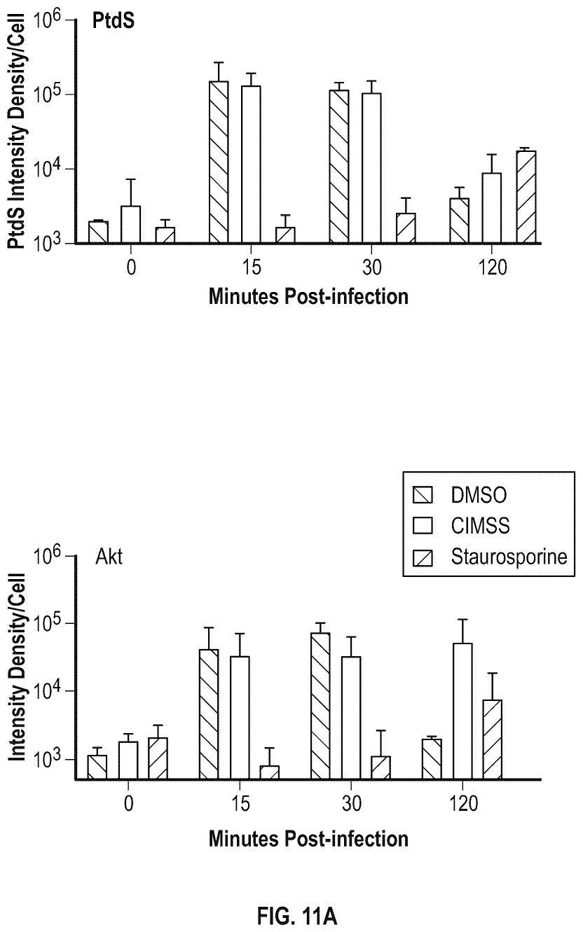

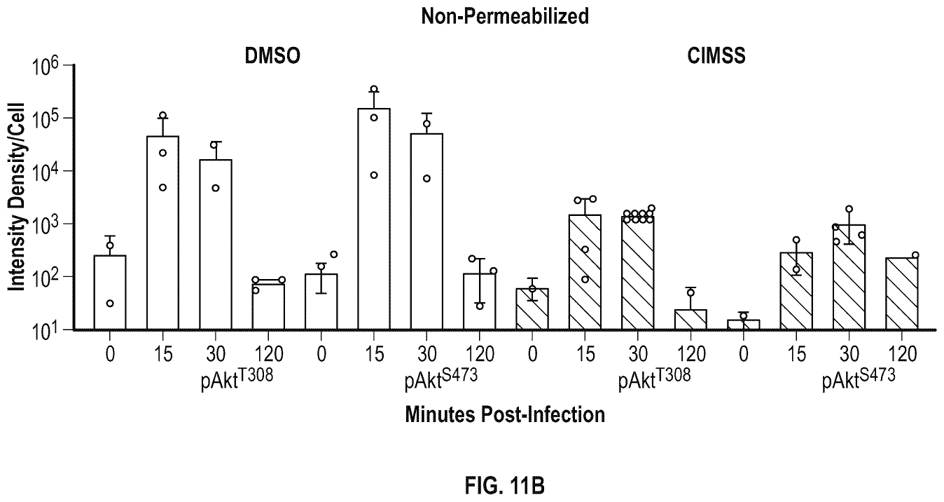

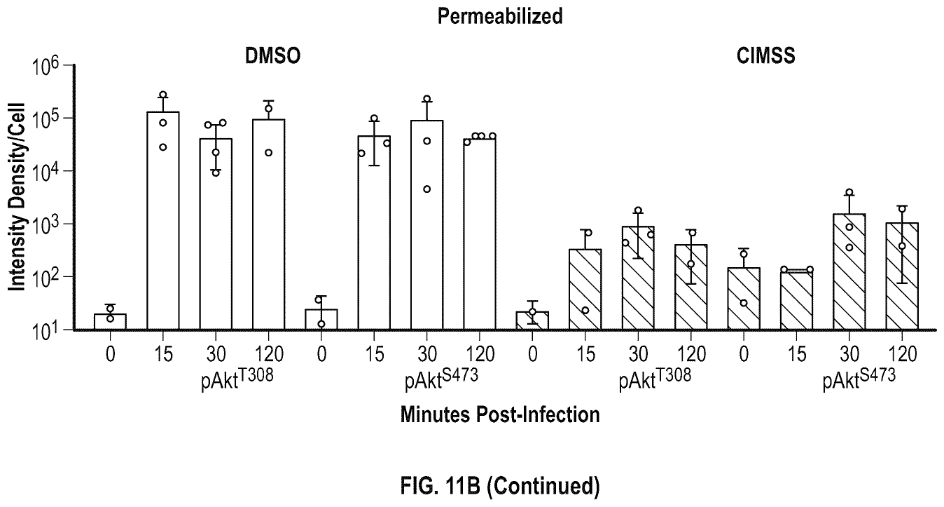

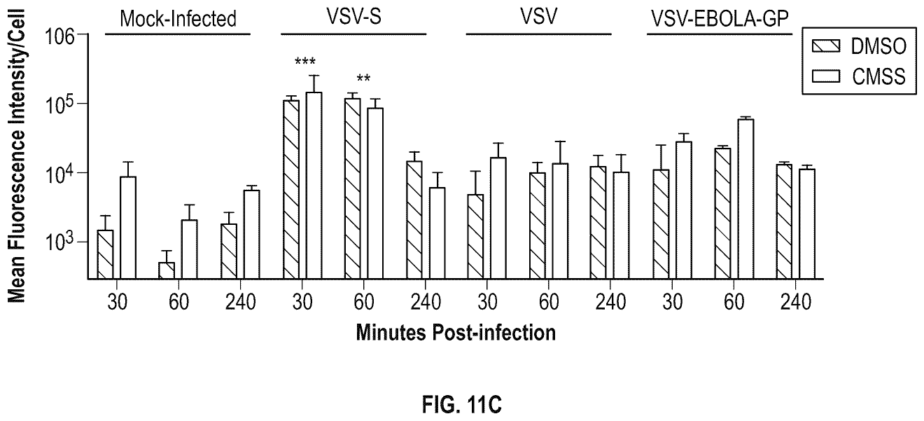

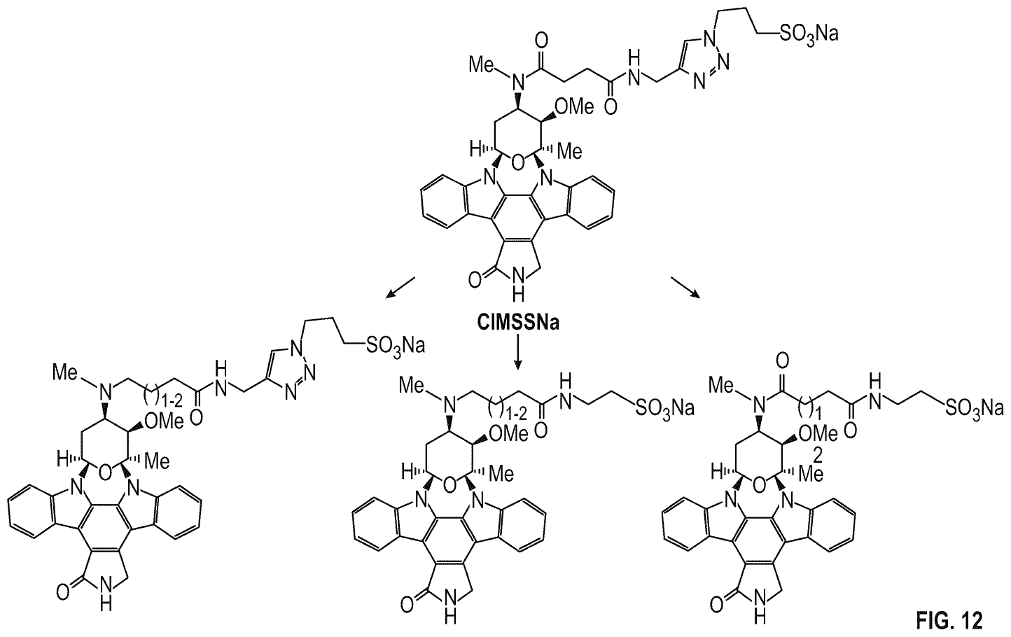

CIMSS did not block HSV binding to cells (FIG. 4A) or the initial Ca²⁺ transient, which is detected within the first three minutes following HSV exposure (FIG. 4B). We previously showed that this initial transient triggers phosphorylation (and activation) of PLSCR1. Phosphorylated PLSCR1 was detected by immunoprecipitation (IP) for PLSCR1 and immunoblotting for phosphorylated proteins (there is no antibody specific for phosphorylated PLSCR1). Phosphorylation of PLSCR1 was inhibited by staurosporine, but not by CIMSS, indicating that PLSCR1 is phosphorylated intracellularly (FIG. 4C). Consistent with this interpretation, only staurosporine inhibited the translocation of PtdS and Akt to the outer leaflet of the plasma membrane (FIG. 4D and FIG. 11). PtdS and Akt were detected by microscopy in non-permeabilized cells within 15 minutes of exposure to HSV but were no longer detected on the cell exterior at 120 minutes in the DMSO treated cells, consistent with the previously described kinetics and restoration of the membrane lipid distribution in response to HSV entry. CIMSS did not block translocation of PtdS and Akt to the outer leaflet but did reduce levels of phosphorylated Akt (serine 473 and threonine 308) detected in non-permeabilized cells, indicating that these phosphorylation events (unlike PLSCR1 phosphorylation) occur extracellularly (FIG. 4E and FIG. 11). Treatment with CIMSS also resulted in a significant reduction in the extended Ca²⁺ release (quantified for the first hour pi) (FIG. 4B) as well as a decrease in phosphorylated Akt detected in permeabilized cells (FIG. 4E)—findings reflective of previous observations that these cellular responses are triggered by viral entry (Cheshenko, N et al., 2007, Mol Biol. of the Cell 18, 3119-3130). Inhibition of Akt phosphorylation at the outer leaflet was associated with a reduction in HSV entry as demonstrated by quantifying viral capsids 1 h and 4 h post-exposure (FIG. 8B). Notably, Akt (but not PtdS) was still detected in non-permeabilized cells at 120 minutes in CIMSS but not DMSO treated cells (FIG. 4D). Using a small molecule PLSCR1 inhibitor, we previously demonstrated that PtdS relocalization is a PLSCR1-dependent process (Cheshenko, N et al., 2018, PLoS Pathog 14, e1006766). The differences in the repartitioning of PtdS and Akt might reflect different internalization mechanisms. Akt colocalizes with HSV glycoprotein B in coimmunoprecipitation studies and potentially could be retained on the outside if viral entry does not occur whereas reinternalization of PtdS may occur independent of viral entry.

### Example 3: HSV Also Triggers Translocation of Pdpk1 and PLCγ and Their Subsequent Phosphorylation is Inhibited by CIMSS

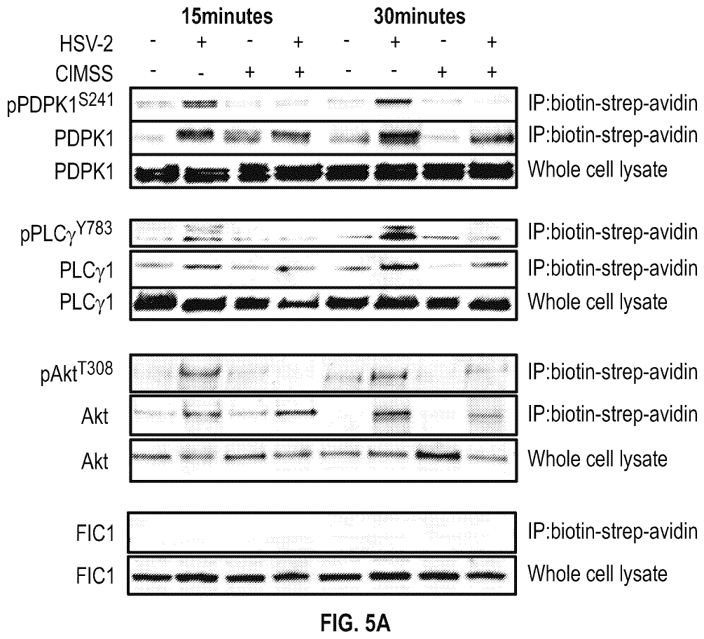

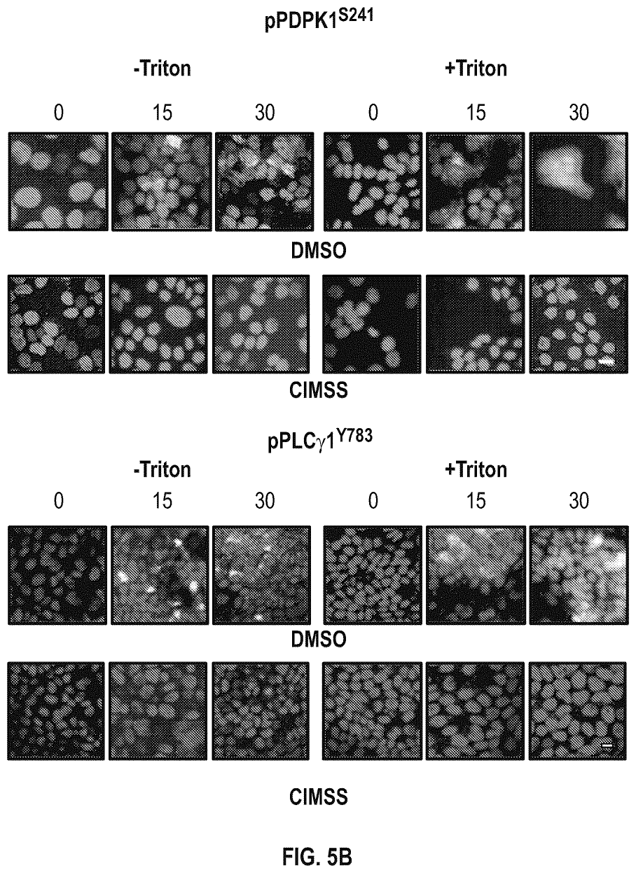

We hypothesized that other kinases typically associated with the inner leaflet of the plasma membrane might also translocate to the outer leaflet of the plasma membrane in response to PLSCR1 activation and be susceptible to the inhibitory effects of CIMSS. We focused on PDPK1, which activates Akt and other kinases, and PLCγ, which is involved in activating intracellular Ca²⁺ signaling pathways and may be a substrate for phosphorylated Akt (Cheshenko, N. et al., 2003, J Cell Biol 163, 283-293; Heras-Martinez G. et al., 2019, Scientific Reports 9, 14527; Wang, Y et al., 2006, Mol Biol. of the Cell 17, 2267-2277). Prior to HSV exposure, neither PDPK1, PLCγ or Akt were detected in the membrane fraction following biotinylation of cell surface proteins, precipitation with streptavidin beads, and immunoblotting the precipitated proteins with antibodies for the respective proteins. The proteins were detected in the whole cell lysates. However, within 15 minutes of HSV exposure, total and phosphorylated PDPK1, PLCγ and Akt were detected in the membrane fraction and their phosphorylation was reduced when the cells were treated with CIMSS (FIG. 5A). As an additional control, blots were probed for FIC-1 (floppase), a cytosolic protein, which was only detected in the cellular lysates, but not in the membrane fraction. Similar results were obtained by confocal microscopy. No pPDPK1 or pPLCγ signal was detected in cells prior to HSV exposure but both phosphorylated proteins were detected in images obtained 15 and 30 minutes following viral infection, and this was inhibited by CIMSS (FIG. 5B).

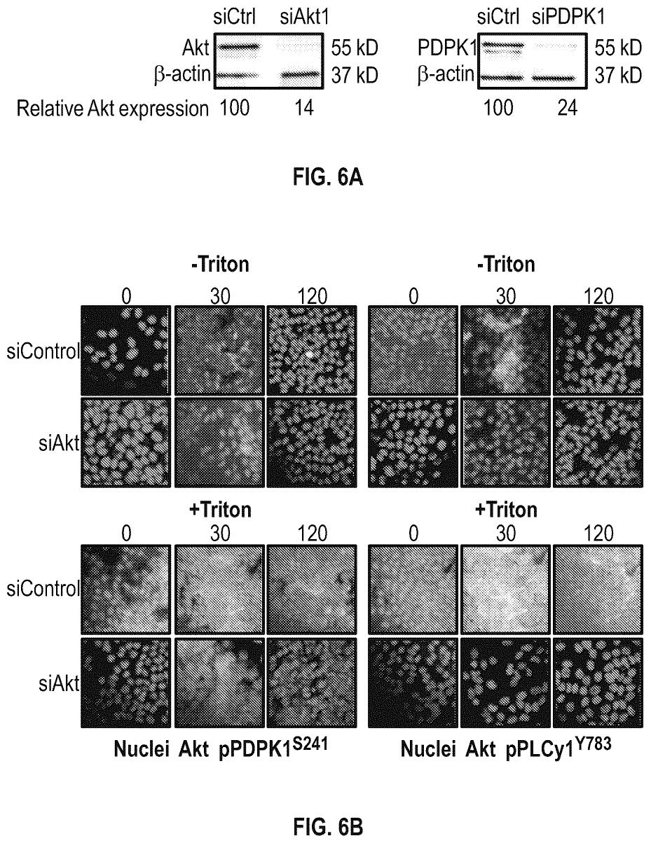

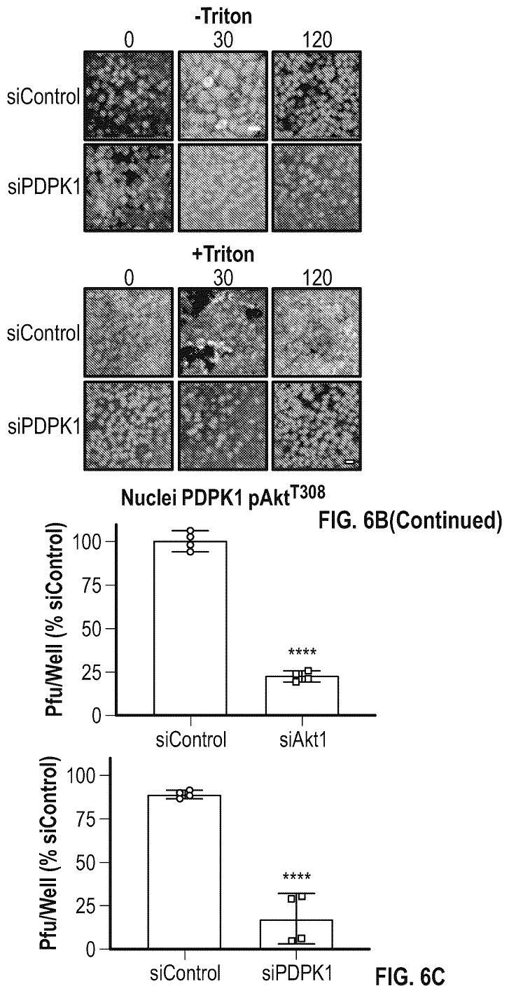

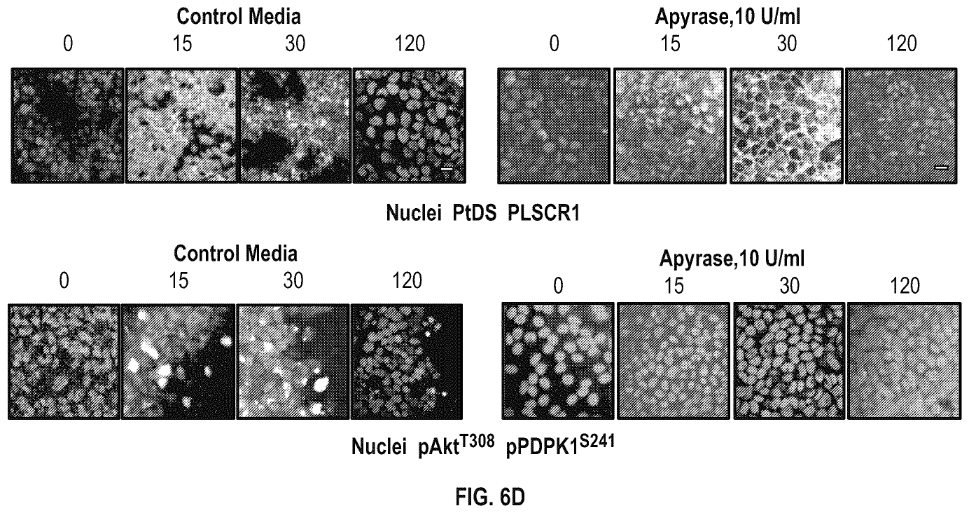

Transfection of HaCat cells with siRNA targeting Akt1 (the dominant form) or PDPK1 reduced protein expression as assayed by immunoblots to 14% and 24%, respectively, relative to cells transfected with a control siRNA (FIG. 6A). The silencing of Akt1 had no effect on HSV-triggered phosphorylation of PDPK1 but resulted in a reduction in phosphorylated PLCγ in non-permeabilized cells. Conversely, silencing of PDPK1 was associated with a reduction in phosphorylated Akt (FIG. 6B). These findings demonstrate that PDPK1 and PLCγ are also translocated to the outer leaflet of the plasma membrane in response to HSV. They further suggest, that similar to intracellular signaling pathways, PDPK1 is activated upstream and PLCγ downstream of Akt phosphorylation when these kinases are translocated to the outer leaflet of the plasma membrane. Silencing of Akt and PDPK1 were each associated with a significant reduction in HSV infection by plaque assay (FIG. 6C). To further assess the importance of these extracellular ATP-dependent kinases on HSV infection, we conducted studies in the presence of apyrase, a cell-impermeable enzyme that hydrolyzes extracellular ATP to AMP. Apyrase had no discernible effect on the detection of PtdS or PLSCR1 at the outer leaflet by fluorescence microscopy, consistent with intracellular activation of PLSCR1, but inhibited the HSV-induced phosphorylation of Akt and PDPK1 in non-permeabilized cells (FIG. 6D).

### Example 4: CIMSS Blocks Viral Entry Mediated By SARS-COV-2 Spike Protein

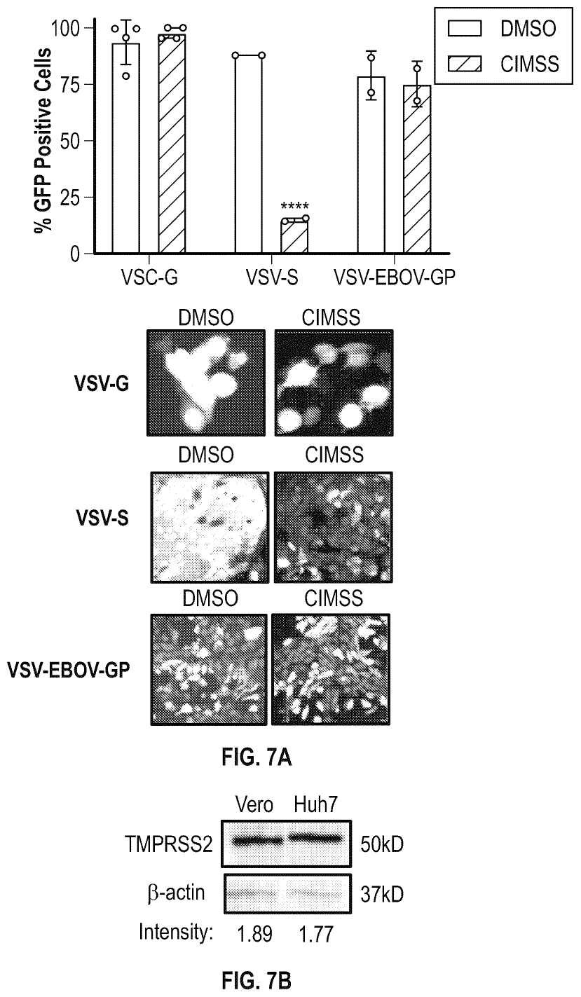

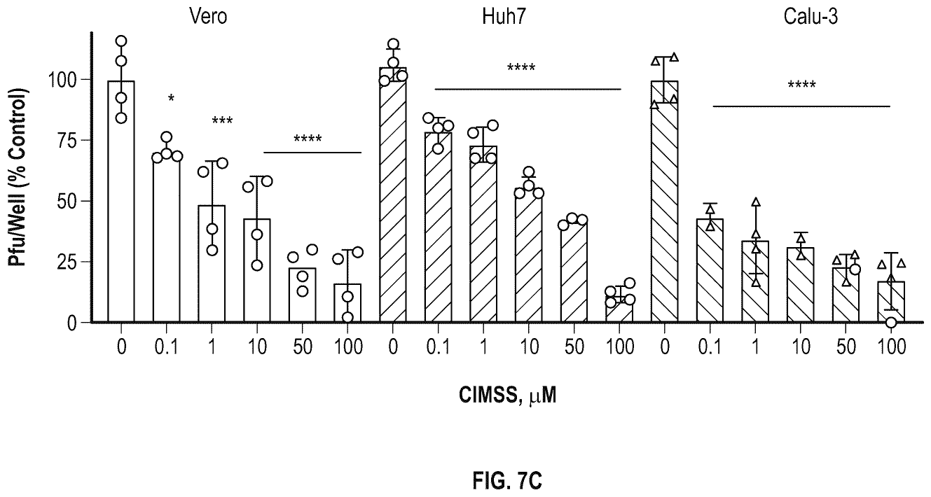

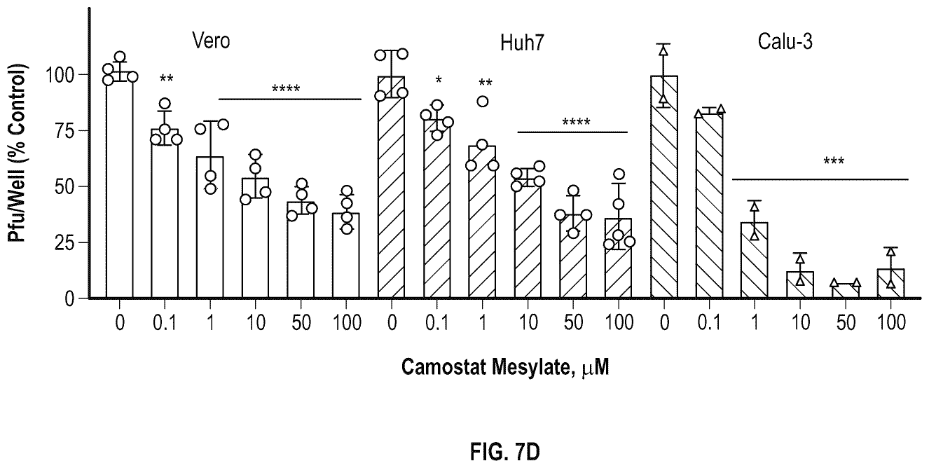

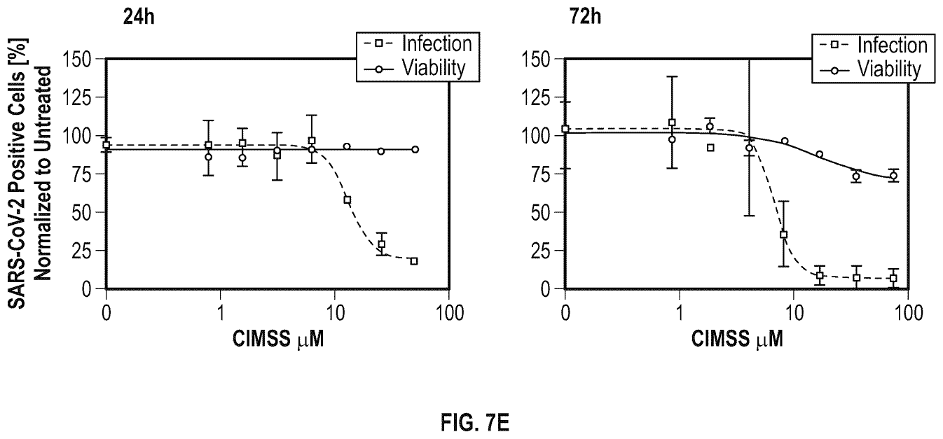

In addition to providing a tool to identify kinases that are phosphorylated at the outer leaflet of the plasma membrane, susceptibility to CIMSS may identify other viruses that exploit PLSCR1-dependent signaling pathways to promote viral entry. To test this hypothesis, we evaluated the inhibitory effects of CIMSS on vesicular stomatitis viruses (VSV) expressing native glycoprotein G (VSV-G), the Ebola virus glycoprotein (VSV-EBOV-GP) or SARS-CoV-2 spike protein (VSV-S); each of these also express enhanced GFP for tracking (Mulherkar et al., 2011, Virology 419, 72-83; Dieterle, ME et al., 2020, Cell Host Microbe 28, 486-496). VSV and EBOV enter cells by endocytosis (Das, D K et al., 2020, PLoS Biol 18, e3000626; Albertini et al., 2012, Viruses 4, 117-139), whereas SARS-CoV-2 enters both by fusion of the viral envelope and cell plasma membrane as well as by endocytosis (Hoffman, M. et al., 2020, Cell 181, 271-280; Hoffman et al., 2020, Nature 585, 588-590). CIMSS (10 μM) had no effect on VSV-G or VSV-EBOV-GP, but significantly reduced VSV-S infection of Vero cells as monitored by quantifying the percentage of GFP+ cells (FIG. 7A). We extended the studies with VSV-S to include the human cell lines, Huh7 and Calu-3. SARS-CoV-2 infection of Calu-3 cells has been shown to be highly dependent on expression of the cellular protease TMPRSS2, which triggers the cleavage of spike to release the S2 fusion subunit and is insensitive to chloroquine, an inhibitor of endocytosis. Western blots demonstrated that Huh7 and Vero cells also express TMPRSS2. Both CIMSS and camostat mesylate, a protease inhibitor that blocks TMPRSS2 activity, individually inhibited VSV-S infection of all 3 cell lines (Calu-3, Vero and Huh7) in a dose dependent manner (FIGS. 7C and 7D). Importantly, CIMSS exhibited similar inhibitory activity against authentic SARS-CoV-2 (WA1/2020) infection of human Huh-7.5 at 24 and 72 h. The percentage of infected cells was determined by automated microscopy after staining for viral nucleoprotein to identify infected cells and for total cell number by nuclear staining with Hoechst (FIG. 7E).

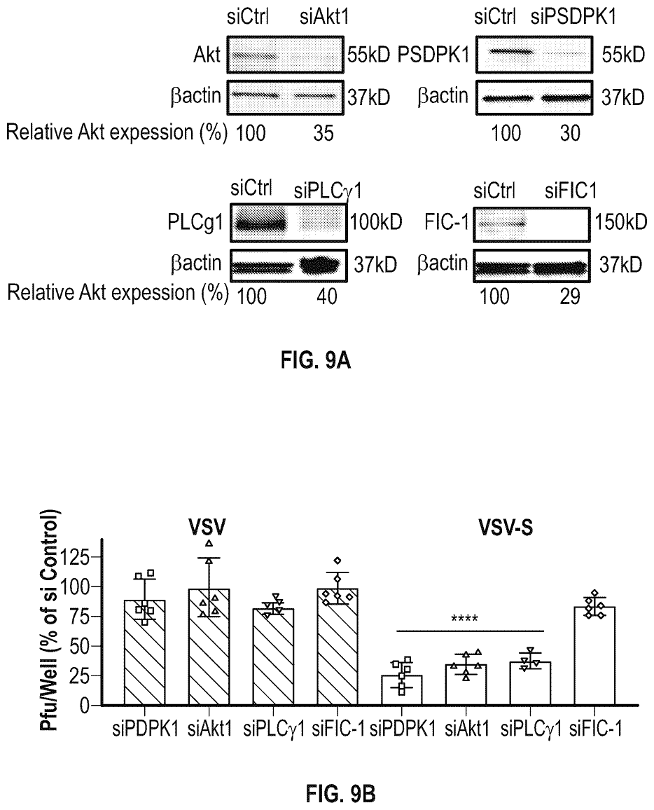

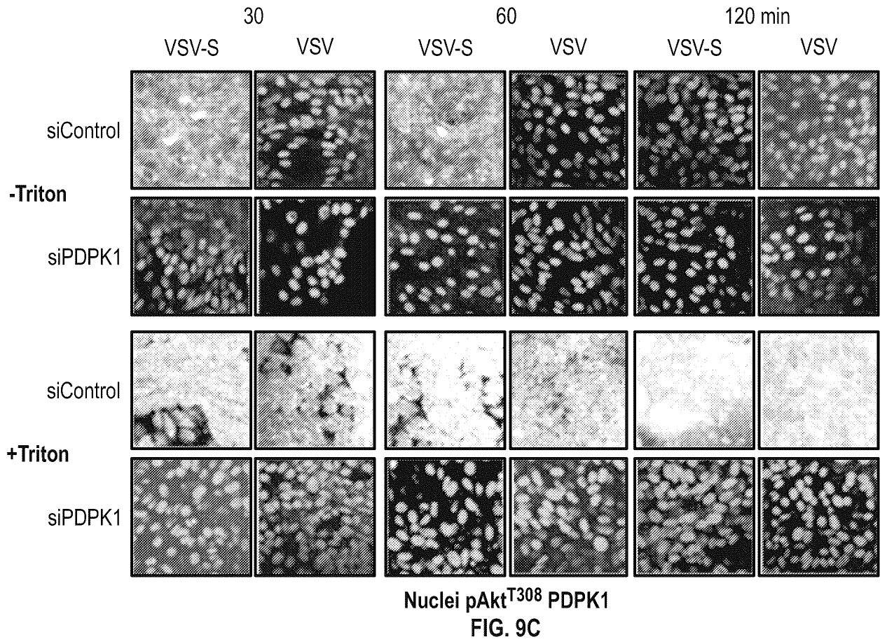

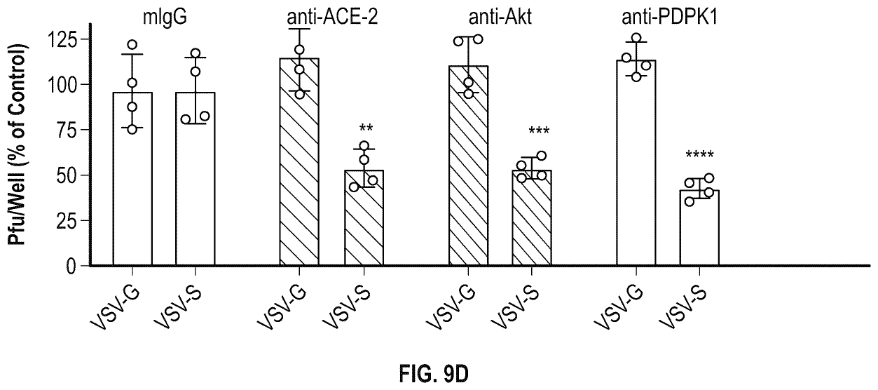

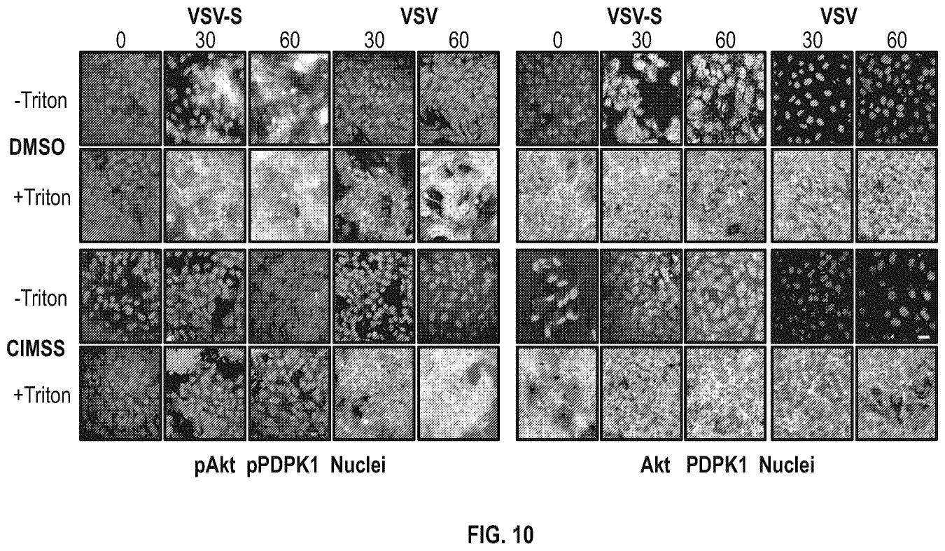

To directly address the role of the PLSCR1 on entry mediated by the SARS-CoV-2 spike protein, we assessed phosphorylation of immunoprecipitated PLSCR1 following exposure of Vero cells to the VSV pseudotyped viruses. As observed with HSV, VSV-S, but not VSV-G or VSV-EBOV-GP triggered, phosphorylation of PLSCR1. Phosphorylation of PLSCR1 was preserved in the presence of CIMSS, but was inhibited by staurosporine, as well as by treatment with anti-ACE2 or anti-Spike antibodies (FIG. 8A). VSV-S triggered PLSCR1 phosphorylation was associated with translocation of PtdS to the outer leaflet as assessed by confocal microscopy, which peaked at 30 minutes with restoration of membrane architecture by 4 hours (FIG. 8B). Moreover, VSV-S(but not native VSV-G or VSV-EBOV-GP) triggered Akt translocation and phosphorylation as assessed following biotinylation of cell surface proteins, precipitation with streptavidin beads, and immunoblotting for pAktT308 or total Akt; the extracellular phosphorylation of Akt was inhibited by CIMSS (FIG. 8C). Phosphorylation of Akt was detected 30 and 60 minutes post exposure to VSV-S and was inhibited by CIMSS. Similar results were obtained with confocal microscopy; Akt and phosphorylated Akt (pAkt) as well as PDPK1 and phosphorylated PDPK1 (pPDPK1) were detected in non-permeabilized cells in response to VSV-S and their phosphorylation was inhibited by CIMSS, (FIG. 9D and FIG. 10).

To evaluate whether this pathway contributed to VSV-S entry, Vero cells were transfected with siRNA targeting Akt, PDPK1, PLCγl, or FIC-1 (negative control). Targeted protein expression was reduced to 29-40% of expression detected in cells transfected with a control siRNA, as judged by Western blot (FIG. 9A). Silencing of PDPK1, Akt1 and PLCγ1 resulted in significant reduction of VSV-S but not VSV-G infection (FIG. 9B). Furthermore, silencing of PDPK1 prevented VSV-S induced phosphorylation of Akt following VSV-S infection as evidenced by microscopy of non-permeabilized and permeabilized cells (FIG. 9C). To further assess the role of this signaling pathway in VSV-S entry, Vero cells were infected with VSV-S or VSV in the presence of antibodies that target Akt, PDKP1, the human ACE2 receptor, or an isotype control for 1 hour, washed, and infection monitored by counting plaques 24 h pi. Anti-ACE2 (murine mAb), anti-Akt (rabbit polyclonal), and anti-PDPK1 (rabbit polyclonal) significantly inhibited VSV-S but not VSV-G infection (FIG. 9D).

### DISCUSSION

We describe the synthesis and characterization of a cell impermeable analog of staurosporine, CIMSS, which represents the prototype of a new class of tool compounds. Impermeability of this analog was demonstrated by its partitioning properties, lack of cytotoxicity, inability to induce apoptosis, failure to inhibit intracellular Akt phosphorylation in response to insulin at concentrations 100-1000-fold greater than the parental drug. The higher concentrations of CIMSS were used in these assays because the analog is generally not as potent and exhibits reduced IC₅₀s relative to staurosporine for a range of kinases. Without being limited by theory, the amide formed by acylation possesses altered hydrogen bonding capabilities relative to the initial secondary amine, which could negatively impact its ability to serve as a hydrogen bond acceptor, and in some cases impose unfavorable sterics. This argument has been invoked to explain why a staurosporine derivative acylated at the 4′-methylamine exhibits an ˜80-fold reduction in affinity for ASK1/MAP3K5 relative to staurosporine (M. Kawaguchi et al., Bioorganic & Medicinal Chemistry Letters, 18, 3752-3755 (2008)), although there is not complete concordance in the literature, as a different acylated staurosporine analog exhibited an IC₅₀ for PKA very similar to staurosporine (H. Shi, et al, Chem Commun (Camb), 47, 11306-11308 (2011)).

Importantly, CIMSS retains the broad inhibitory profile characteristic of staurosporine. This promiscuity, coupled with impermeability, makes CIMSS an ideal tool compound for examining unique kinase-dependent processes that are occurring at the outer leaflet of the plasma membrane or extracellularly. CIMSS enabled the identification of cellular proteins (PDPK1 and PLCγ) that are translocated from the extracellular milieu following activation of PLSCR1 by HSV triggered calcium transients. Moreover, CIMSS also uncovered the ability of SARS-CoV-2 spike protein to activate this PLSCR1-Akt signaling pathway. This process is distinct from the previously described intracellular activation of the phosphidylionistol 3-kinase/Akt signaling pathway, which among other intracellular processes, regulates clathrin-mediated endocytosis of SARS-CoV-2 and other viruses (Basile, M. S., 2022, Drug Discov Today 27, 848-856; Cheng et al., 2015, Cell Microbiol 17, 967-987). However, the intracellular activation of Akt signaling would not be susceptible to CIMSS as illustrated by the studies with insulin. Notably, HIV also triggers the externalization of PtdS to promote membrane fusion through the activation of a different phospholipid scramblase, TMEM16F, but whether this is associated with externalization of cellular kinases and thus whether HIV entry would be inhibited by CIMSS is not yet known (26).

Specifically, using CIMSS to discriminate whether a phosphorylation event occurs intracellularly or in association with the outer leaflet of the plasma membrane, we showed that in response to canonical activation of the insulin receptor, Akt is phosphorylated intracellularly, while HSV or VSV-S infection resulted in extracellular Akt phosphorylation. In contrast, PLSCR1 itself was phosphorylated intracellularly in response to virally induced Ca²⁺ transients as evidenced by its inhibition by unmodified staurosporine and insensitivity to CIMSS. Focusing on proteins associated with intracellular Akt signaling, we demonstrated that PLSCR1 activation also resulted in the translocation of PDPK1 and PLCγ to the outer leaflet of the plasma membrane where they are phosphorylated, as evidenced by confocal imaging, biotinylation of cell membranes, and susceptibility to blockade of these processes by CIMSS. By analogy with the canonical cytoplasmic signaling pathways (N. Cheshenko et al., J Cell Biol 163, 283-293 (2003); G. de 1. Heras-Martinez et al., Scientific Reports 9, 14527 (2019); Y. Wang, et al., Molecular Biology of the Cell, 17, 2267-2277 (2006)), we proposed that PDPK1 participates in extracellular autophosphorylation and that the resulting activated phospho-PDPK1 is likely responsible for the subsequent phosphorylation of outer leaflet Akt since silencing of PDPK1 prevented outer leaflet plasma membrane Akt phosphorylation. Furthermore, activated phosphorylated Akt likely triggers PLCγ phosphorylation, as silencing of Akt results in a reduction in PLCγ phosphorylation at the outer leaflet of the plasma membrane. Notably, CIMSS inhibited PDPK1, Akt and PLCγ phosphorylation and reduced HSV entry and infection, highlighting the importance of extracellular kinase function/phosphorylation events in viral infection. Evidence for a role of extracellular kinase activity in HSV entry is further provided by the studies with apyrase, which inhibited the phosphorylation of Akt and PDPK1, and with our prior studies showing that antibodies targeting Akt inhibit HSV and subsequent infection.

CIMSS also served as an effective tool to identify other viruses that might exploit a similar kinase-dependent signaling pathway to promote viral entry. Specifically, we found that VSV pseudotyped with SARS-CoV-2 spike, but not viruses expressing the native VSV glycoprotein G or EBOV glycoprotein, were susceptible to inhibition by CIMSS. Notably, authentic SARS-CoV-2 was similarly inhibited as VSV-S. As observed with HSV, VSV-S activated phospholipid scramblase) to promote translocation of PtdS, PDPK1 and Akt to the outer leaflet and the latter two kinases were subsequently phosphorylated in a CIMSS-sensitive manner. Silencing of PDPK1, Akt1 or PLCγ resulted in a reduction in VSV-S infection supporting the importance of this signaling pathway. Additional support for the role of extracellular kinases in VSV-S comes from observation that polyclonal or monoclonal Abs targeting Akt or PDPK1 inhibited VSV-S infection. While the antibody studies do not distinguish between mechanisms involving reductions in catalytic activity or steric blockade of protein-protein interactions involving kinases, these results, combined with the observation that CIMSS inhibits VSV-S, provide strong evidence that both Akt and PDPK1 extracellular signaling are directly involved in HSV, VSV-S(and native SARS-CoV-2) entry.

VSV and EBOV primarily enter cells via endocytosis and their entry is effectively blocked by inhibitors of this pathway (D. K. Das et al., PLoS Biol 18, e3000626 (2020); A. A. Albertini, Eet al, Viruses 4, 117-139 (2012)). For example, EBOV enters by macropinocytosis and traffics through the endosomal pathway where cathepsin-dependent cleavage of EBOV-GP occurs. Subsequently, the cleaved viral glycoprotein interacts with Niemann-Pick C1 (NPC1), a late endosome/lysosome resident host protein, to trigger viral and intracellular membrane fusion, resulting in release of the viral genome. While this intracellular fusion step is blocked by inhibitors of receptor tyrosine kinases, which interfere with post-internalization signaling events (C. M. Stewart et al., PLoS Pathog., 17, e1009275 (2021)), we demonstrated this process was insensitive to CIMSS, consistent with the impermeability and extracellular activity of CIMSS. In contrast, HSV-1 and HSV-2 primarily enter human keratinocytes and other epithelial cells by direct fusion between the viral envelope and the plasma membrane, which generates a lipid pore that permits the viral genome (and associated tegument proteins) to be released intracellularly (M. Wittels, et al., Virus Res 18, 271-290 (1991)). This plasma membrane-viral envelope fusion event is blocked by inhibitors of the PLSCR1-Akt signaling (N. Cheshenko, et al., PLoS Pathog 14, e1006766 (2018); N. Cheshenko et al., J Cell Biol 163, 283-293 (2003); N. Cheshenko, et al., Molecular biology of the cell 18, 3119-3130 (2007).; N. Cheshenko et al., FASEB J. 27, 2584-2599 (2013)) pathway, including, as shown here, the cell impermeable inhibitor of Akt phosphorylation, CIMSS, as well as anti-Akt and anti-PDPK1 antibodies. The observation that SARS-CoV-2 is also inhibited by CIMSS and activates a PLSCRJ-Akt signaling pathway similar to that observed for HSV supports the notion that SARS-CoV-2 enters these cells, at least in part, by direct fusion. Although there has been controversy about the relative role of fusion of the viral envelope with plasma membrane versus endocytosis for entry of SARS-CoV-2 into different cell types, our studies support the importance of a direct fusion pathway for viral entry in the cells studied here. We found that the three cell types studied (Vero, Huh7 and Calu-3 cells) were susceptible to inhibition by both CIMSS and camostat mesylate, an inhibitor of transmembrane protease, serin 2 (TMPRS22). The results are consistent with other studies showing that camostat mesylate is more effective than ammonium chloride, which increases endosomal pH to block cathepsin activity, or other inhibitors of endocytosis at inhibiting SARS-CoV-2 infection (M. Hoffmann et al., Cell 181, 271-280 e278 (2020)). It should be noted that CIMSS did not abolish HSV or VSV-S infection, which may reflect the ability of both viruses to use alternative endocytic pathways for entry.

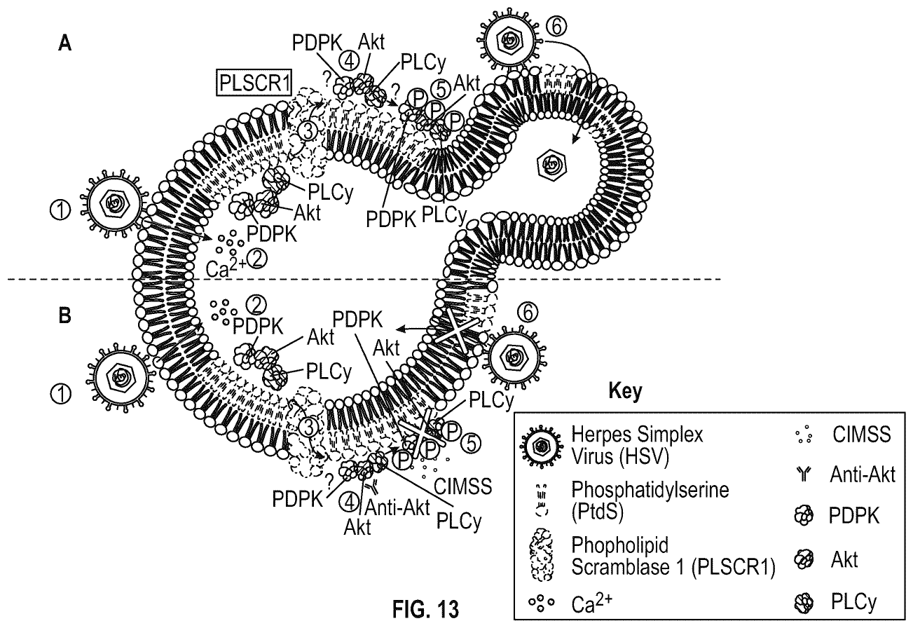

In summary, we have exploited the unique properties of CIMSS to examine viral entry mechanisms of HSV and SARS-CoV-2, and to identify unique extracellular interactions and catalytic contributions to these processes (FIG. 13). Future efforts to define the mechanistic contribution of extracellular kinase activities to viral entry, will benefit from more selective reagents, including mechanistically defined antibodies and impermeable analogs of selective kinase inhibitors. While we focused on CIMSS as a tool compound, our results suggest that cell impermeable kinase inhibitors may also represent a new candidate class of antiviral drugs. Advantages include their safety profile, which reduces off-target engagement, as interactions with cytoplasmic proteins are precluded, and the likelihood that this class of inhibitors is less prone to select for viral escape mutants because they target host proteins and not the virus. The previously unappreciated translocation of kinases to the extracellular milieu and the subsequence activation/phosphorylation of these kinases at the outer leaflet may have implications for other biological processes associated with calcium transients that activate phospholipid scramblase.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity and are to be construed to cover both the singular and the plural, unless otherwise indicated herein or clearly contradicted by context. “Or” means “and/or” unless clearly stated otherwise. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments. A “combination thereof” is open and includes any combination comprising at least one of the listed components or properties optionally together with a like or equivalent component or property not listed

Unless specified to the contrary herein, all test standards are the most recent standard in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A pharmaceutical composition comprising a pharmaceutically acceptable carrier and an amount of a cell-impermeable inhibitor comprising a cell-impermeable kinase inhibitor, a cell-impermeable Akt inhibitor, an anti-Akt antibody, a cell-impermeable 3-phosphoinositide-dependent protein kinase-1 (PDPK1) inhibitor, an anti-PDPK1 antibody, or a combination thereof for inhibiting or treating a coronavirus infection in a subject or for preventing or treating a disease caused by a coronavirus infection in a subject.

2. The pharmaceutical composition of claim 1, wherein the cell impermeable inhibitor comprises a cell-impermeable moiety covalently linked to an Akt inhibitor, a 3-phosphoinositide-dependent protein kinase-1 inhibitor, wherein the cell-impermeable moiety has the structure of Formula 1: 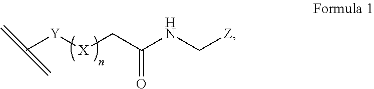
where Y is a bond, carbonyl, or methylene (—CH₂—), X is methylene or polyethylene glycol, n is 0-10, and Z is —C₁-C₈alkylSO₃⁻, —C₁-C₈alkylSO₄, —C₁-C₈alkylSO₂H, —C₁-C₈alkylPO₄, —C₁-C₈alkylPO(OH)₂, —C₁-C₈alkylPO₃NH₂, or a 5- or 6-membered heterocycle substituted with one of —C₁-C₈alkylSO₃⁻, —C₁-C₈alkylSO₄, —C₁-C₅alkylSO₂H, —C₁-C₈alkylPO₄, —C₁-C₈alkylPO(OH)₂, and —C₁-C₈alkylPO₃NH₂, and optionally substituted with one or more of C₁-C₂alkyl, C₁-C₂alkoxy, halogen, and hydroxyl, and [symbol] indicates the point of attachment to the cell-impermeable inhibitor.

3. The pharmaceutical composition of claim 2, wherein Formula 1 is

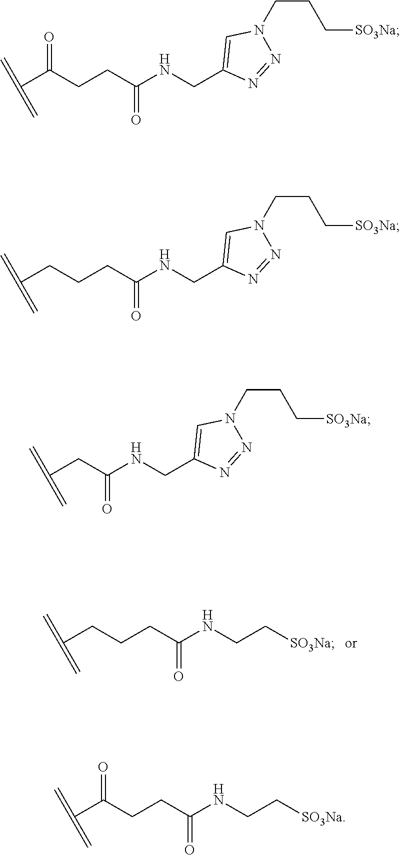

4. The pharmaceutical composition of claim 2, wherein the cell-impermeable inhibitor has the structure of Formula 2 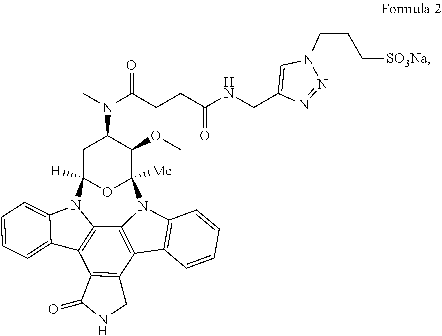
or a pharmaceutically acceptable salt or solvate thereof.

5. A method of preventing or treating a coronavirus infection in a subject or preventing or treating a disease caused by a coronavirus infection in a subject, comprising administering to the subject an effective amount of a cell-impermeable inhibitor comprising a cell-impermeable kinase inhibitor, a cell-impermeable Akt inhibitor, an anti-Akt antibody, a cell-impermeable 3-phosphoinositide-dependent protein kinase-1 (PDPK1) inhibitor, an anti-PDPK1 antibody, or a combination thereof.

6. The method of claim 5, wherein the coronavirus is a SARS Co-V, SARS-CoV-2, or a MERS-CoV virus.

7-8. (canceled)

9. The method of claim 6, wherein the cell-impermeable inhibitor comprises a cell-impermeable moiety covalently linked to an Akt inhibitor, a 3-phosphoinositide-dependent protein kinase-1 inhibitor, or a combination thereof.

10. The method of claim 9, wherein the cell-impermeable moiety has the structure of Formula 1: 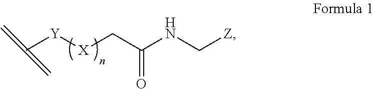
where Y is a bond, carbonyl, or methylene (—CH₂—), X is methylene or polyethylene glycol, n is 0-10, and Z is —C₁-C₈alkylSO₃⁻, —C₁-C₈alkylSO₄, —C₁-C₈alkylSO₂H, —C₁-C₈alkylPO₄, —C₁-C₈alkylPO(OH)₂, —C₁-C₈alkylPO₃NH₂, or a 5- or 6-membered heterocycle substituted with one of —C₁-C₈alkylSO₃⁻, —C₁-C₈alkylSO₄, —C₁-C₈alkylSO₂H, —C₁-C₈alkylPO₄, —C₁-C₈alkylPO(OH)₂, and —C₁-C₈alkylPO₃NH₂, and optionally substituted with one or more of C₁-C₂alkyl, C₁-C₂alkoxy, halogen, and hydroxyl, and where [symbol] indicates the point of attachment to the cell-impermeable inhibitor.

11. The method of claim 10, wherein Formula 1 is

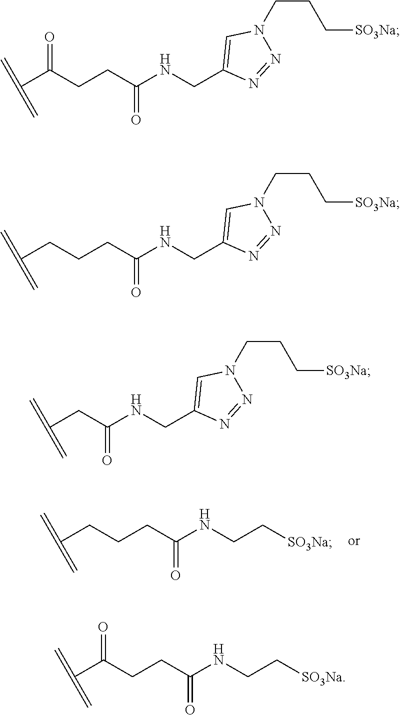

12. The method of claim 9, wherein the cell impermeable inhibitor has the structure of Formula 2, or a pharmaceutically acceptable salt thereof:

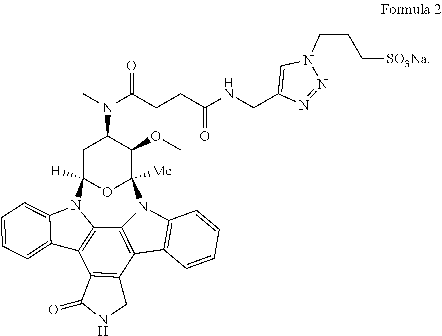

13. The method of claim 5, wherein the cell-impermeable inhibitor is in a pharmaceutical composition.

14. The method of claim 13, wherein the cell-impermeable inhibitor is administered systemically to the subject.

15. The method of claim 14, wherein the cell-impermeable inhibitor is administered to the subject orally, parenterally, or nasally.

16. A method of preventing or treating a condition, a disorder or a disease associated with a SARS-CoV-2 infection in a subject comprising administering to the subject an effective amount of a cell-impermeable inhibitor comprising a cell-impermeable kinase inhibitor, a cell-impermeable Akt inhibitor, a cell-impermeable 3-phosphoinositide-dependent protein kinase-1 inhibitor, or a combination thereof.

17. (canceled)

18. The method of claim 16, wherein the cell-impermeable inhibitor comprises a cell-impermeable moiety covalently linked to an Akt inhibitor, a 3-phosphoinositide-dependent protein kinase-1 inhibitor, or a combination thereof.

19. The method of claim 18, wherein the cell-impermeable moiety has the structure of Formula 1: 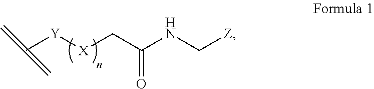
where Y is a bond, carbonyl, or methylene (—CH₂—), X is methylene or polyethylene glycol, n is 0-10, and Z is —C₁-C₈alkylSO₃⁻, —C₁-C₈alkylSO₄, —C₁-C₈alkylSO₂H, —C₁-C₈alkylPO₄, —C₁-C₈alkylPO(OH)₂, —C₁-C₈alkylPO₃NH₂, or a 5- or 6-membered heterocycle substituted with one of —C₁-C₈alkylSO₃⁻, —C₁-C₅alkylSO₄, —C₁-C₅alkylSO₂H, —C₁-C₈alkylPO₄, —C₁-C₈alkylPO(OH)₂, and —C₁-C₈alkylPO₃NH₂, and optionally substituted with one or more of C₁-C₂alkyl, C₁-C₂alkoxy, halogen, and hydroxyl, and [symbol] indicates the point of attachment to the cell-impermeable inhibitor.

20. The method of claim 19, wherein Formula 1 is

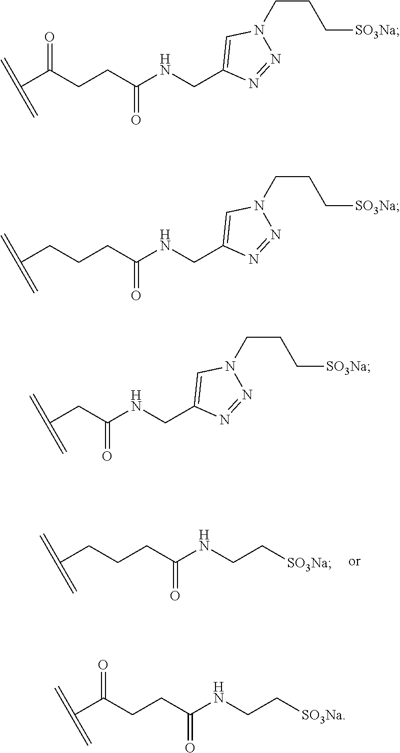

21. The method of claim 16, wherein the cell-impermeable inhibitor has the structure of Formula 2, or a pharmaceutically acceptable salt thereof:

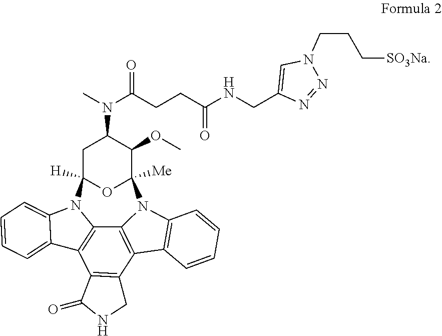

22. A method of preventing or treating a herpes simplex virus infection in a subject or preventing or treating a disease caused by a herpes simplex virus infection in a subject, comprising administering to the subject an effective amount of a cell-impermeable inhibitor comprising a cell-impermeable kinase inhibitor, a cell-impermeable Akt inhibitor, an anti-Akt antibody, a cell-impermeable 3-phosphoinositide-dependent protein kinase-1 (PDPK1) inhibitor, an anti-PDPK1 antibody, or a combination thereof.

23. (canceled)

24. The method of claim 22, wherein the herpes simplex virus is herpes simplex virus-1 or herpes simplex virus-2.

25. (canceled)
